(19) United States
(12) Patent Application Publication
(10) Pub. No.: US 2026/0176373 A1
(43) Pub. Date: Jun. 25, 2026

(54) ANTI-HER2/TROP2 ANTIBODIES AND USES THEREOF

(71) Applicant: BRIGHT BIOLOGICS LLC, Lewes, DE (US)

(72) Inventors: Ming Yang, Mountain View, CA (US); Hongyan Zhong, Mountain View, CA (US)

(21) Appl. No.: 19/127,252

(22) PCT Filed: Nov. 30, 2023
(86) PCT No.: PCT/US2023/081841

§ 371 Date: May 5, 2025

Related U.S. Application Data
(60) Provisional application No. 63/429,216, filed on Dec. 1, 2022.

(51) Int. Cl. C07K 16/32; A61K 47/68; A61P 35/00; C07K 16/30; G01N 33/575

(52) U.S. Cl. CPC C07K 16/32; A61K 47/68031; A61K 47/6879; A61P 35/00; C07K 16/30; G01N 33/5759; C07K 2317/31; C07K 2317/524; C07K 2317/526; C07K 2317/53; C07K 2317/565; C07K 2317/569; G01N 2333/82

(57) ABSTRACT

This disclosure relates to anti-HER2 antibodies or antigen binding fragments thereof, anti-TROP2 antibodies or antigen binding fragments thereof, antigen-binding protein constructs (e.g., bispecific antibodies or antigen-binding fragments thereof) that specifically bind to two different antigens (e.g., HER2 and TROP2), and antibody drug conjugates.

### TECHNICAL FIELD

This disclosure relates to antibodies or antigen-binding fragments thereof, antigen-binding protein constructs (e.g., bispecific antibodies), and antibody drug conjugates.

### SEQUENCE LISTING

The instant application contains a Sequence Listing which has been submitted herewith and is hereby incorporated by reference in its entirety. Said .xml copy, created on Nov. 28, 2023 is named 52501-0006WO1, and is 41,620 bytes in size.

### BACKGROUND

Human epidermal growth factor receptor 2 (HER2) (also known as ERBB2) is a transmembrane receptor belonging to the epidermal growth factor receptor subfamily of receptor protein tyrosine kinases. HER2 is overexpressed in various cancer types such as breast cancer and gastric cancer and has been reported to be a negative prognostic factor in breast cancer.

Trophoblast cell-surface antigen 2 (TROP2), also known as Tumor-associated calcium signal transducer 2 (TACSTD2), is a cell surface glycoprotein encoded and expressed by the TACSTD2 gene. TROP2 is a protein closely related to tumors. It mainly promotes tumor cell growth, proliferation and metastasis by regulating calcium ion signaling pathways, cyclin expression, and reducing fibronectin adhesion. Studies have found that TROP2 protein is highly expressed in breast cancer, colon cancer, bladder cancer, gastric cancer, pancreatic cancer, oral squamous cell carcinoma and ovarian cancer. The protein can promote tumor cell proliferation, invasion, metastasis, spread and other processes.

Considering the important roles of HER2 and TROP2 in cancer, there is a need to develop a therapeutic agent targeting HER2 and/or TROP2.

### SUMMARY

This disclosure relates to anti-HER2 antibodies or antigen binding fragments thereof, anti-TROP2 antibodies or antigen binding fragments thereof, antigen-binding protein constructs (e.g., bispecific antibodies or antigen-binding fragments thereof) that specifically bind to two different antigens (e.g., HER2 and TROP2), and antibody drug conjugates involving these antibodies or antigen binding fragments thereof.

In one aspect, the disclosure is related to an antibody or antigen-binding fragment thereof that binds to HER2 (human epidermal growth factor receptor 2), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence,
wherein the selected VHH CDRs 1, 2, 3 amino acid sequences are set forth in SEQ ID NOs: 22, 23, and 24, respectively.

In some embodiments, the VHH comprises CDRs 1, 2, 3 with the amino acid sequences set forth in SEQ ID NOs: 22, 23, and 24, respectively.

In one aspect, the disclosure is related to an antibody or antigen-binding fragment thereof that binds to HER2 comprising a heavy-chain antibody variable domain (VHH) comprising an amino acid sequence that is at least 80% identical to a selected VHH sequence, wherein the selected VHH sequence is set forth in SEQ ID NO: 1.

In some embodiments, the antibody or antigen-binding fragment specifically binds to HER2.

In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof described herein.

In some embodiments, the antibody or antigen-binding fragment comprises a human IgG Fc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof described herein.

In one aspect, the disclosure is related to an antibody or antigen-binding fragment thereof that binds to TROP2 (Tumor-associated calcium signal transducer 2), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 25, 26, and 27, respectively;
(2) the selected VHH CDRs 1, 2, 3 amino acid sequences are set forth in SEQ ID NOs: 28, 29, and 30, respectively; and
(3) the selected VHH CDRs 1, 2, 3 amino acid sequences are set forth in SEQ ID NOs: 31, 32, and 33, respectively.

In some embodiments, the VHH comprises CDRs 1, 2, 3 with the amino acid sequences set forth in SEQ ID NOs: 25, 26, and 27, respectively.

In some embodiments, the VHH comprises CDRs 1, 2, 3 with the amino acid sequences set forth in SEQ ID NOs: 28, 29, and 30, respectively.

In some embodiments, the VHH comprises CDRs 1, 2, 3 with the amino acid sequences set forth in SEQ ID NOs: 31, 32, and 33, respectively.

In one aspect, the disclosure is related to an antibody or antigen-binding fragment thereof that binds to TROP2 comprising a heavy-chain antibody variable domain (VHH) comprising an amino acid sequence that is at least 80% identical to a selected VHH sequence, wherein the selected VHH sequence is selected from the group consisting of SEQ ID NOs: 2-12.

In some embodiments, the VHH comprises the sequence of SEQ ID NO: 2.

In some embodiments, the VHH comprises the sequence of SEQ ID NO: 3.

In some embodiments, the VHH comprises the sequence of SEQ ID NO: 4.

In some embodiments, the VHH comprises the sequence of SEQ ID NO: 10.

In some embodiments, the VHH comprises the sequence of SEQ ID NO: 12.

In some embodiments, the antibody or antigen-binding fragment specifically binds to TROP2.

In some embodiments, the antibody or antigen-binding fragment is a humanized antibody or antigen-binding fragment thereof.

In one aspect, the disclosure is related to an antibody or antigen-binding fragment thereof comprising the VHH CDRs 1, 2, 3, of the antibody or antigen-binding fragment thereof described herein.

In some embodiments, the antibody or antigen-binding fragment comprises a human IgGFc.

In some embodiments, the antibody or antigen-binding fragment comprises two or more heavy-chain antibody variable domains.

In one aspect, the disclosure is related to an antibody or antigen-binding fragment thereof that cross-competes with the antibody or antigen-binding fragment thereof described herein.

In one aspect, the disclosure is related to a multi-specific antibody or antigen-binding fragment thereof, comprising a first VHH (VHH1) that specifically binds to HER2, a second VHH (VHH2) that specifically binds to TROP2.

In some embodiments, the multi-specific antibody or antigen-binding fragment thereof further comprises a third VHH (VHH3) that specifically binds to HER2, and a fourth VHH (VHH4) that specifically binds to TROP2.

In some embodiments, the VHH1 and/or the VHH3 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 33.

In some embodiments, the VHH1 and/or the VHH3 comprises an amino acid sequence that is at least 80% identical to a selected VHH sequence, wherein the selected VHH sequence is set forth in SEQ ID NO: 1.

In some embodiments, the VHH2 and/or the VHH4 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 34.

In some embodiments, the VHH3 and the VHH4 comprise an amino acid sequence that is at least 80% identical to a selected VHH sequence, wherein the selected VHH sequence is selected from the group consisting of SEQ ID NOs: 2-12.

In some embodiments, the multi-specific antibody or antigen-binding fragment thereof comprises a human IgG Fc.

In some embodiments, the VHH1 and VHH3 are linked to the N-terminus or the C-terminus of the human IgG Fc.

In some embodiments, the VHH2 and VHH4 are linked to the N-terminus or the C-terminus of the human IgG Fc.

In one aspect, the disclosure is related to a polypeptide complex, comprising
(a) a first polypeptide comprising from N-terminus to C-terminus: a first heavy-chain antibody variable domain (VHH1), a first hinge region, a first CH2, a first CH3, and a second VHH (VHH2); and
(b) a second polypeptide comprising from N-terminus to C-terminus: a third VHH (VHH3), a second hinge region, a second CH2, a second CH3, and a fourth VHH (VHH4),
wherein the VHH1 and the VHH3 specifically bind to HER2, and the VHH2 and the VHH4 specifically bind to TROP2.

In some embodiments, the first polypeptide comprises a sequence that is at least 80% identical to SEQ ID NO: 18; and/or wherein the second polypeptide comprises a sequence that is at least 80% identical to SEQ ID NO: 18.

In some embodiments, the VHH2 is linked to the C-terminus of the first CH2 and first CH3 via a first linker peptide sequence.

In some embodiments, the VHH4 is linked to the C-terminus of the second CH2 and second CH3 via a second linker peptide sequence.

In some embodiments, the first and/or the second linker peptide sequence is at least 80% identical to SEQ ID NO: 34 or 35.

In one aspect, the disclosure is related to a polypeptide complex, comprising
(a) a first polypeptide comprising from N-terminus to C-terminus: a VHH1, a VHH2, a first hinge region, a first CH2, and a first CH3; and
(b) a second polypeptide comprising from N-terminus to C-terminus: a VHH3, a VHH4, a second hinge region, a second CH2, and a second CH3,
wherein the VHH1 and the VHH3 specifically bind to HER2 and the VHH2 and the VHH4 specifically bind to TROP2.

In some embodiments, the VHH1 is linked to the N-terminus of the VHH2 via a first linker peptide sequence.

In some embodiments, the VHH3 is linked to the N-terminus of the VHH4 via a second linker peptide sequence.

In some embodiments, the first polypeptide comprises a sequence that is at least 80% identical to SEQ ID NO: 17; and/or wherein the second polypeptide comprises a sequence that is at least 80% identical to SEQ ID NO: 17.

In some embodiments, the first and/or the second linker peptide sequence is at least 80% identical to SEQ ID NO: 34 or 35.

In one aspect, the disclosure is related to a polypeptide complex, comprising
(a) a first polypeptide comprising from N-terminus to C-terminus: a VHH2, a VHH1, a first hinge region, a first CH2, and a first CH3; and
(b) a second polypeptide comprising from N-terminus to C-terminus: a VHH4, a VHH3, a second hinge region, a second CH2, and a second CH3,
wherein the VHH1 and the VHH3 specifically bind to HER2 and the VHH2 and the VHH4 specifically bind to TROP2.

In some embodiments, the VHH2 is linked to the N-terminus of the VHH1 via a first linker peptide sequence.

In some embodiments, the VHH4 is linked to the N-terminus of the VHH3 via a second linker peptide sequence.

In some embodiments, the first polypeptide comprises a sequence that is at least 80% identical to SEQ ID NO: 15 or 21; and/or wherein the second polypeptide comprises a sequence that is at least 80% identical to SEQ ID NO: 15 or 21.

In some embodiments, the first and/or the second linker peptide sequence is at least 80% identical to SEQ ID NO: 34 or 35.

In one aspect, the disclosure is related to a polypeptide complex, comprising
(a) a first polypeptide comprising from N-terminus to C-terminus: a VHH2, a first hinge region, a first CH2, a first CH3, and VHH1; and
(b) a second polypeptide comprising from N-terminus to C-terminus: a VHH4, a second hinge region, a second CH2, a second CH3, and a VHH3,
wherein the VHH1 and the VHH3 specifically bind to HER2, and the VHH2 and the VHH4 specifically bind to TROP2.

In some embodiments, the first polypeptide comprises a sequence that is at least 80% identical to SEQ ID NO: 16 or 20; and/or wherein the second polypeptide comprises a sequence that is at least 80% identical to SEQ ID NO: 16 or 20.

In some embodiments, the VHH1 is linked to the C-terminus of the first CH2 and first CH3 via a first linker peptide sequence.

In some embodiments, the VHH3 is linked to the C-terminus of the second CH2 and second CH3 via a second linker peptide sequence.

In some embodiments, the first and/or the second linker peptide sequence is at least 80% identical to SEQ ID NO: 34 or 35.

In some embodiments, the VHH1 and/or the VHH3 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 33.

In some embodiments, the VHH1 and/or the VHH3 comprises an amino acid sequence that is at least 80% identical to a selected VHH sequence, wherein the selected VHH sequence is set forth in SEQ ID NO: 1.

In some embodiments, the VHH2 and/or the VHH4 comprise complementarity determining regions (CDRs) 1, 2, and 3, wherein the CDR1 region comprises an amino acid sequence that is at least 80% identical to a selected CDR1 amino acid sequence, the CDR2 region comprises an amino acid sequence that is at least 80% identical to a selected CDR2 amino acid sequence, and the CDR3 region comprises an amino acid sequence that is at least 80% identical to a selected CDR3 amino acid sequence; wherein the selected CDRs 1, 2, and 3 amino acid sequences are listed in FIG. 34.

In some embodiments, the VHH2 and/or the VHH4 comprise an amino acid sequence that is at least 80% identical to a selected VHH sequence, wherein the selected VHH sequence is selected from the group consisting of SEQ ID NOs: 2-12.

In one aspect, the disclosure is related to a nucleic acid comprising a polynucleotide encoding the antibody or antigen-binding fragment thereof described herein, the multi-specific antibody or antigen-binding fragment thereof described herein, or the polypeptide complex described herein.

In some embodiments, the nucleic acid is a DNA (e.g., cDNA) or RNA (e.g., mRNA).

In one aspect, the disclosure is related to a vector comprising one or more of the nucleic acids described herein.

In one aspect, the disclosure is related to a cell comprising the vector described herein.

In some embodiments, the cell is a CHO cell.

In one aspect, the disclosure is related to a cell comprising one or more of the nucleic acids described herein.

In one aspect, the disclosure is related to a method of producing an antibody or an antigen-binding fragment thereof, the method comprising
(a) culturing the cell described herein under conditions sufficient for the cell to produce the antibody or the antigen-binding fragment; and
(b) collecting the antibody or the antigen-binding fragment produced by the cell.

In one aspect, the disclosure is related to a T-cell engager (TCE) comprising the antibody or antigen-binding fragment thereof described herein, the multi-specific antibody or antigen-binding fragment thereof described herein, or the polypeptide complex described herein.

In one aspect, the disclosure is related to a chimeric antigen receptor (CAR) comprising the antibody or antigen-binding fragment thereof described herein, the multi-specific antibody or antigen-binding fragment thereof described herein, or the polypeptide complex described herein.

In one aspect, the disclosure is related to a CAR-T, CAR-NK or CAR-NKT cell comprising the CAR described herein.

In one aspect, the disclosure is related to an antibody-drug conjugate comprising the antibody or antigen-binding fragment thereof described herein, the multi-specific antibody or antigen-binding fragment thereof described herein, or the polypeptide complex described herein, covalently bound to a therapeutic agent.

In some embodiments, the therapeutic agent is a cytotoxic or cytostatic agent.

In some embodiments, the drug-to-antibody ratio (DAR) is 4.

In one aspect, the disclosure is related to a method of treating a subject having cancer, the method comprising administering a therapeutically effective amount of a composition comprising the antibody or antigen-binding fragment thereof described herein, the multi-specific antibody or antigen-binding fragment thereof described herein, the polypeptide complex described herein, the TCE described herein, the CAR described herein, the CAR-T or CAR-NK or CAR-NKT cell described herein, or the antibody-drug conjugate described herein, to the subject.

In some embodiments, the subject has a cancer expressing HER2.

In some embodiments, the subject has a cancer expressing TROP2.

In some embodiments, the cancer is gastric cancer, cervical cancer, esophagus cancer, thyroid carcinoma, cholangiocarcinoma, colon cancer, rectum cancer, lung cancer, breast cancer, kidney cancer, hepatocellular carcinoma, renal cancer, endometrial carcinoma, pancreatic cancer, head and neck cancer or late-stage solid tumor.

In some embodiments, the cancer is non-small cell lung cancer (NSCLC).

In one aspect, the disclosure is related to a method of decreasing the rate of tumor growth, the method comprising contacting a tumor cell with an effective amount of a composition comprising the antibody or antigen-binding fragment thereof described herein, the multi-specific antibody or antigen-binding fragment thereof described herein, the polypeptide complex described herein, the TCE described herein, the CAR described herein, the CAR-T or CAR-NK or CAR-NKT cell described herein, or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, the multi-specific antibody or antigen-binding fragment thereof described herein, the polypeptide complex described herein, the TCE described herein, the CAR described herein, the CAR-T or CAR-NK or CAR-NKT cell described herein, or the antibody-drug conjugate described herein.

In one aspect, the disclosure is related to a pharmaceutical composition comprising the antibody or antigen-binding fragment thereof described herein, the multi-specific antibody or antigen-binding fragment thereof described herein, the polypeptide complex described herein, the TCE described herein, the CAR described herein, the CAR-T or CAR-NK or CAR-NKT cell described herein, or the antibody-drug conjugate described herein, and a pharmaceutically acceptable carrier.

In one aspect, the disclosure is related to an engineered antibody or antigen-binding fragment thereof, comprising a serine residue at heavy chain position 220, according to EU numbering.

In one aspect, the disclosure is related to an antibody-drug conjugate, comprising an engineered hinge region covalently bound to a therapeutic agent, wherein the engineered hinge region comprises a C220S mutation, wherein the therapeutic agent is bound to the cysteine residue at position 226 or 229, according to EU numbering.

In one aspect, the disclosure is related to an antibody-drug conjugate (ADC), comprising an engineered hinge region covalently bound to a therapeutic agent, wherein the engineered hinge region comprises a serine at position 220, wherein the therapeutic agent is bound to the cysteine residue at position 226 or 229, according to EU numbering.

In some embodiments, the drug-to-antibody ratio (DAR) is 1-4.

In some embodiments, the ADC further comprises a VHH, wherein the VHH is linked to the engineered hinge region.

In some embodiments, the ADC comprises:
(1) (a) a first polypeptide comprising from N-terminus to C-terminus: a VHH1, a first hinge region, a first CH2, and a first CH3; and (b) a second polypeptide comprising from N-terminus to C-terminus: a VHH2, a second hinge region, a second CH2, a second CH3;
(2) (a) a first polypeptide comprising from N-terminus to C-terminus: a VHH1, VHH2, a first hinge region, a first CH2, and a first CH3; and (b) a second polypeptide comprising from N-terminus to C-terminus: a VHH3, VHH4, a second hinge region, a second CH2, a second CH3; or
(3) (a) a first polypeptide comprising from N-terminus to C-terminus: a VHH1, a first hinge region, a first CH2, a first CH3, and a VHH2; and (b) a second polypeptide comprising from N-terminus to C-terminus: a VHH3, a second hinge region, a second CH2, a second CH3, a VHH4.

In one aspect, the disclosure is related to an antibody-drug conjugate (ADC), comprising an engineered hinge region covalently bound to a therapeutic agent, wherein the therapeutic agent is bound to the cysteine residue at position 220, 226, or 229, according to EU numbering.

In some embodiments, the drug-to-antibody ratio (DAR) is 4-6.

In some embodiments, the ADC comprises:
(1) (a) a first polypeptide comprising from N-terminus to C-terminus: a VHH1, a first hinge region, a first CH2, and a first CH3; and (b) a second polypeptide comprising from N-terminus to C-terminus: a VHH2, a second hinge region, a second CH2, a second CH3;
(2) (a) a first polypeptide comprising from N-terminus to C-terminus: a VHH1, VHH2, a first hinge region, a first CH2, and a first CH3; and (b) a second polypeptide comprising from N-terminus to C-terminus: a VHH3, VHH4, a second hinge region, a second CH2, a second CH3; or
(3) (a) a first polypeptide comprising from N-terminus to C-terminus: a VHH1, a first hinge region, a first CH2, a first CH3, and a VHH2; and (b) a second polypeptide comprising from N-terminus to C-terminus: a VHH3, a second hinge region, a second CH2, a second CH3, a VHH4.

In one aspect, the disclosure is related to a polypeptide complex, comprising (a) a first polypeptide comprising from N-terminus to C-terminus: a VHH1, a first hinge region, a first CH2, and a first CH3; and (b) a second polypeptide comprising from N-terminus to C-terminus: a VHH2, a second hinge region, a second CH2, and a second CH3.

In one aspect, the disclosure is related to a polypeptide complex, comprising (a) a first polypeptide comprising from N-terminus to C-terminus: a VHH1, a first hinge region, a first CH2, a first CH3, and VHH2; and (b) a second polypeptide comprising from N-terminus to C-terminus: a VHH3, a second hinge region, a second CH2, a second CH3, and a VHH4.

In one aspect, the disclosure is related to a polypeptide complex, comprising (a) a first polypeptide comprising from N-terminus to C-terminus: a VHH1, a VHH2, a first hinge region, a first CH2, a first CH3; and (b) a second polypeptide comprising from N-terminus to C-terminus: a VHH3, a VHH4, a second hinge region, a second CH2, and a second CH3.

In some embodiments, the VHH1 and the VHH3 specifically bind to a first tumor antigen, and the VHH2 and the VHH4 specifically bind to a second tumor antigen.

In one aspect, the disclosure is related to an antibody-drug conjugate (ADC) comprising the polypeptide complex described herein covalently bound to a payload.

In some embodiments, the payload is selected from the group consisting of cytotoxic agents, cytostatic agents, radionuclides, biologically active proteins, synthetic polymers, enzymes, nucleic acids (e.g. DNA, or RNA) and fragments thereof.

In some embodiments, each of the first hinge region and the second hinge region consists of 2 cysteines.

In some embodiments, each of the first hinge region and the second hinge region consists of 3 cysteines.

In some embodiments, each of the first hinge region and the second hinge region consists of 2 cysteines, wherein the drug-to-antibody ratio (DAR) is 1.0-4.0.

In some embodiments, each of the first hinge region and the second hinge region consists of 3 cysteines, wherein the drug-to-antibody ratio (DAR) is 4.0-6.0.

In one aspect, the disclosure is related to a method of diagnosing a disease or condition, wherein the method comprises incubating a sample with a composition comprising the antibody or antigen-binding fragment thereof described herein, the multi-specific antibody or antigen-binding fragment thereof described herein, or the polypeptide complex described herein.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “antigen-binding protein construct” is (i) a single polypeptide that includes at least one antigen-binding domain or (ii) a complex of two or more polypeptides (e.g., the same or different polypeptides) that together form at least one or more different antigen-binding domains. Non-limiting examples and aspects of antigen-binding protein constructs are described herein. Additional examples and aspects of antigen-binding protein constructs are known in the art. In some embodiments, the antigen-binding protein construct has 1, 2, 3, 4, 5, 6, 7, 8, or more than 8 antigen-binding domains.

As used herein, the term “antigen-binding domain” refers to one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by a VH-VL dimer. In some embodiments, the antigen-binding domain is a VHH. Non-limiting examples of antigen-binding domains are described herein. Additional examples of antigen-binding domains are known in the art. In some examples, an antigen-binding domain can bind to a single antigen.

As used herein, the term “bispecific antibody” refers to an antibody that binds to two different epitopes. The epitopes can be on the same antigen or on different antigens.

As used herein, the term “multispecific antibody” refers to an antibody that binds to two or more different epitopes. The epitopes can be on the same antigen or on different antigens.

As used herein, a “VHH” refers to the variable domain of a heavy chain antibody or a single domain antibody. In some embodiments, the VHH is a humanized VHH.

As used herein, when referring to an antibody, the phrases “specifically binding” and “specifically binds” mean that the antibody interacts with its target molecule (e.g., HER2) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a HER2 molecule may be referred to as a HER2-specific antibody or an anti-HER2 antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A, 1B, & 1C show whole cell binding (WCB) of top VHH-Fc clones to human HER2 stably transfected CHO cells (CHO-h-HER2 stable cells, FIG. 1A), SKBR3 cells (breast cancer cell, FIG. 1B) and N87 cells (stomach cancer cell, FIG. 1C) as determined by flow cytometry with AlexaFluor488 conjugated anti-human IgG Fc as a secondary antibody. X axis is values of antibody concentration in nanomolar. Y axis is values of Median Fluorescence Intensity (MFI) of AlexaFluor 488. Herceptin and Perjeta are reference anti-HER2 antibodies from Roche. 4A8, 11F6 & 11D5 are anti-human HER2 VHH-Fc clones.

FIG. 2A shows ELISA binding of top 3 VHH-Fc clones to human HER2-Domain IV. 96-well plates were coated with human HER2-Domain IV-his protein at 2.5 μg/ml overnight, and binding of anti-HER2 VHH-Fc clones 4A8, 11F6 & 11D5, and reference anti-HER2 antibodies Herceptin and Perjeta (from Roche) was detected by anti-human IgG Fc-HRP. X axis is values of antibody concentration in nanomolar. Y axis is values of OD at 450 nm.

FIG. 2B shows ELISA binding of VHH-Fc clones 11F6 & 11D5 to human HER2-Domain II+III+IV. A 96-well plate was coated with human HER2-Domain II+III+IV-his protein at 3 μg/ml overnight, and binding of anti-HER2 VHH-Fc clone 11F6 & 11D5 was detected by anti-human IgG Fc-HRP. X axis is values of antibody concentration in nanomolar. Y axis is values of OD at 450 nm.

FIG. 2C shows ELISA binding of VHH-Fc clones 11F6 & 11D5 to human HER2-Domain II. A 96-well plate was coated with human HER2-Domain II-his protein at 3 μg/ml overnight, and binding of anti-HER2 VHH-Fc clone 11F6 & 11D5 was detected by anti-human IgG Fc-HRP. X axis is values of antibody concentration in nanomolar. Y axis is values of OD at 450 nm.

FIGS. 3A & 3B show anti-HER2 antibodies mediated killing of cancer cells with high HER2 expression in the presence of human PBMCs. Anti-HER2 VHH-Fc clones 4A8, 11F6 & 11D5, and reference antibody Perjeta were added in different concentrations to AU-565 and SKBR3 cells in 96-well plates seeded 1 day ago at 15×10³/well. Fresh human PBMCs were added at 0.3×10⁶/well (1:20 ratio of tumor cell: PBMC), and cell viability was measured 1 day later by adding CCK-8 and reading at OD 450 nm. X axis is values of antibody concentration in nanomolar. Y axis is values of OD at 450 nm.

FIG. 4 shows the effects of anti-HER2 antibodies on proliferation of AU-565 cells. Anti-HER2 VHH-Fc clones 4A8, 11F6 & 11D5, and reference antibody Herceptin were added in different concentrations to AU-565 cells in 96-well plates seeded 1 day ago at 15×10³/well in RPMI1640 culture medium containing 2% FBS. Cell viability was measured 1 day later by adding CCK-8 and reading at OD 450 nm. X axis is values of antibody concentration in nanomolar. Y axis is values of OD at 450 nm.

FIGS. 5A & 5B show whole cell binding of TROP2 VHH-Fc clones to human TROP2 stably transfected CHO cells (CHO-h-TROP2 stable cells, FIG. 5A) and mouse TROP2 stably transfected 293T cells (FIG. 5B) as determined by flow cytometry, with AlexaFluor488 conjugated anti-human IgG Fc as a secondary antibody. X axis is values of antibody concentration in nanomolar. Y axis is values of Median Fluorescence Intensity of AlexaFluor 488. Immu-132 is a reference anti-TROP2 antibody from Gilead. 8B5, 1E9, 1H11, 4D3, 5G11, 4C6, 8F10, 3H9, 6B7 and 1H6 are anti-human TROP2 VHH-Fc clones.

FIG. 6 shows whole cell binding of lead TROP2 VHH-Fc clones to endogenously expressed TROP2 in SKBR3 cells as determined by flow cytometry, with AlexaFluor488 conjugated anti-human IgG Fc as a secondary antibody. X axis is values of antibody concentration in nanomolar. Y axis is values of Median Fluorescence Intensity of AlexaFluor 488. Immu-132 is a reference anti-TROP2 antibody from Gilead.

FIG. 7 shows competition of lead anti-TROP2 VHH-Fc antibodies to Immu-132 in whole cell binding. CHO-h-TROP2 stable cells were pre-incubated with anti-TROP2 VHH-Fc antibodies at 60 nM for 30 min. After washing Immu-132 was added at different concentrations and incubated for 30 min. Binding was detected by adding AlexaFluor488 conjugated goat anti-human IgG F(ab)2 fragment specific secondary antibody. X axis is values of Immu-132 concentration in nanomolar. Y axis is values of Median Fluorescence Intensity of AlexaFluor 488. 4C6, 3H9 & 1H11 are anti-TROP2 VHH-Fc clones. Negative control (Ctrl) is an anti-human PD-L1 VHH-Fc antibody.

FIG. 8 shows whole cell binding of humanized variants of 3H9 VHH-Fc clone to CHO-h-TROP2 stable cells, as determined by flow cytometry with AlexaFluor488 conjugated anti-human IgG Fc as a secondary antibody. X axis is values of antibody concentration in nanomolar. Y axis is values of Median Fluorescence Intensity of AlexaFluor 488. 3H9 is the Alpaca VHH-Fc clone, 3H9-hv1, hv2, hv3, & hv4 are 4 different humanized variants.

FIGS. 9A, 9B, & 9C show whole cell binding of humanized variants of 4C6 VHH-Fc clone to CHO-h-TROP2 stable cells, determined by flow cytometry with AlexaFluor488 conjugated anti-human IgG Fc as a secondary antibody. X axis is values of antibody concentration in nanomolar. Y axis is values of Median Fluorescence Intensity of AlexaFluor 488. 4C6 is the Alpaca VHH-Fc clone, 4C6-hv1, hv2, hv3, hv4, hv5, hv6, hv7, hv8, hv9 & hv10 are 10 different humanized variants. Immu-132 is a reference anti-TROP2 antibody from Gilead.

FIGS. 10A & 10B show whole cell binding of humanized variants of 1H11 VHH-Fc clone to CHO-h-TROP2 stable cells, as determined by flow cytometry with AlexaFluor488 conjugated anti-human IgG Fc as a secondary antibody. X axis is values of antibody concentration in nanomolar. Y axis is values of Median Fluorescence Intensity of AlexaFluor 488. 1H11 is the Alpaca VHH-Fc clone, 1H11-hv2, hv3, hv4, hv5, hv6, hv7, hv8, hv9 & hv10 are 9 different humanized variants. Immu-132 is a reference anti-TROP2 antibody from Gilead.

FIGS. 11A & 11B show whole cell binding of humanized variants of 1H11 VHH-Fc clone to endogenously expressed TROP2 in NCI-H1975 cells (FIG. 11A) and Colo205 cells (FIG. 11B), as determined by flow cytometry with AlexaFluor488 conjugated anti-human IgG Fc as a secondary antibody. X axis is values of antibody concentration in nanomolar. Y axis is values of Median Fluorescence Intensity of AlexaFluor 488. 1H11 is the Alpaca VHH-Fc clone, 1H11-hv6, hv7, hv8, hv9 & hv10 are 5 different humanized variants. Immu-132 is a reference anti-TROP2 antibody from Gilead.

FIGS. 12A-12F show schematic diagrams of monospecific VHH-Fc constructs (12A & 12B) and BsAb constructs in different formats (12C-12F).

FIGS. 13A-13B show whole cell binding of monospecific anti-HER2 (11D5) or anti-TROP2 (1H11-hv10) and bispecific anti-HER2/TROP2 VHH-Fc clones in different formats to CHO-h-HER2 stable cells (13A) and CHO-h-TROP2 stable cells (13B), as determined by flow cytometry with AlexaFluor488 conjugated anti-human IgG Fc as a secondary antibody. X axis is values of antibody concentration in nanomolar. Y axis is values of Median Fluorescence Intensity of AlexaFluor 488. Constructs names are shown in the figures.

FIGS. 14A-14C show schematic diagrams of monospecific VHH-Fc construct 1H11-hv8 (14A) and BsAb constructs in two different formats (14B & 14C).

FIGS. 15A-15E show the binding affinities of BsAb 1H11-hv8_Fc_11D5 or monospecific Abs to h-HER2 ECD or h-TROP2 ECD as determined by BLI binding assay. Briefly, sensor was loaded with an antibody at 300 nM, then dipped to a solution containing 200 nM of recombinant protein of h-HER2 ECD (his-tagged), after 130s association, sensor moved and dipped to another solution containing 200 nM of h-TROP2 (his-tagged). Y axis is shift in nm, X axis is time in second.

FIG. 16A shows whole cell binding of monospecific anti-HER2 (11D5) or anti-TROP2 (1H11-hv8) and bispecific anti-HER2/TROP2 VHH-Fc clones in different formats to SKBR3 cells, as determined by flow cytometry with AlexaFluor488 conjugated anti-human IgG Fc as a secondary antibody. Antibody names are shown in the figure. Immu-132 is an anti-TROP2 reference antibody from Gilead. Herceptin is an anti-HER2 reference antibody from Roche. X axis is values of antibody concentration in nanomolar. Y axis is values of Median Fluorescence Intensity of AlexaFluor 488.

FIG. 16B shows anti-HER2 or anti-TROP2 monospecific or bispecific antibodies mediated killing of SKBR3 cells in the presence of a vc-MMAE conjugated secondary antibody (a Fab) against human IgG Fc. The viable cells were measured by adding cell counting-8 (CCK-8) reagent and reading at OD 450 nm. Antibody names are shown in the figure. X axis is values of antibody concentration in nanomolar. Y axis is values of OD at 450 nm.

FIG. 17A shows whole cell binding of monospecific anti-HER2 (11D5) or anti-TROP2 (1H11-hv8) and bispecific anti-HER2/TROP2 VHH Fc clones in different formats to NCI-H441 cells, as determined by flow cytometry with AlexaFluor488 conjugated anti-human IgG Fc as a secondary antibody. Antibody names are shown in the figure. X axis is values of antibody concentration in nanomolar. Y axis is values of Median Fluorescence Intensity of AlexaFluor 488.

FIG. 17B shows anti-HER2 or anti-TROP2 monospecific or bispecific antibodies mediated killing of NCI-H441 cells in the presence of a vc-MMAE conjugated secondary antibody (a Fab) against human IgG Fc. The viable cells were measured by adding cell counting-8 (CCK-8) reagent and reading at OD 450 nm. Antibody names are shown in the figure. X axis is values of antibody concentration in nanomolar. Y axis is values of OD at 450 nm.

FIG. 18A shows whole cell binding of monospecific anti-HER2 (11D5) or anti-TROP2 (1H11-hv8) and bispecific anti-HER2/TROP2 VHH Fc clones in different formats to HCC202 cells, as determined by flow cytometry with AlexaFluor488 conjugated anti-human IgG Fc as a secondary antibody. Antibody names are shown in the figure. X axis is values of antibody concentration in nanomolar. Y axis is values of Median Fluorescence Intensity of AlexaFluor 488.

FIG. 18B shows anti-HER2 or anti-TROP2 monospecific or bispecific antibodies mediated killing of HCC202 cells in the presence of a vc-MMAE conjugated secondary antibody (a Fab) against human IgG Fc. The viable cells were measured by adding cell counting-8 (CCK-8) reagent and reading at OD 450 nm. Antibody names are shown in the figure. X axis is values of antibody concentration in nanomolar. Y axis is values of OD at 450 nm.

FIGS. 19A, 20A, 21A, 22A, and 23A show the HIC-HPLC results of 11D5_MMAE (19A), 1H11_MMAE (20A), BsAb_MMAE (1H11-hv8_Fc_11D5_MMAE, 21A), Herceptin_MMAE (22A), and Immu-132_MMAE (23A).

FIGS. 19B, 20B, 21B, 22B, and 23B show the SEC-HPLC results of 11D5_MMAE (19B), 1H11_MMAE (20B), BsAb_MMAE (1H11-hv8_Fc_11D5_MMAE, 21B), Herceptin_MMAE (22B), and Immu-132_MMAE (23B).

FIG. 24A shows the effects of Immu-132_MMAE in different DARs (DAR3.5, 4.0 & 4.4) in killing of NCI-H441 cells. The viable cells were measured by adding cell counting-8 (CCK-8) reagent and reading at OD 450 nm. X axis is values of antibody concentration in nanomolar. Y axis is values of OD at 450 nm.

FIG. 24B shows the effects of Herceptin_MMAE in different drug-to-antibody ratios (DARs) (DAR3.6, 4.0 & 4.3) in killing of AU-565 cells. The viable cells were measured by adding cell counting-8 (CCK-8) reagent and reading at OD 450 nm. X axis is values of antibody concentration in nanomolar. Y axis is values of OD at 450 nm.

FIGS. 25A-25B show whole cell binding of Herceptin and Immu-132 to AU-565 cells (25A) & N87 cells (25B), as determined by flow cytometry with AlexaFluor488 conjugated anti-human IgG Fc as a secondary antibody. X axis is values of antibody concentration in nanomolar. Y axis is values of Median Fluorescence Intensity of AlexaFluor 488.

FIGS. 26A-26C show the effects of vc-MMAE conjugated anti-HER2 (11D5_MMAE & Herceptin_MMAE) or anti-TROP2 (1H11_MMAE & Immu-132_MMAE) monospecific antibodies or anti-HER2/TROP2 BsAb (1H11-hv8_Fc_11D5_MMAE) in killing SKBR3 cells (26A), AU-565 cells (26B) & N87 cells (26C). The viable cells were measured by adding cell counting-8 (CCK-8) reagent and reading at OD 450 nm. X axis is values of antibody concentration in nanomolar. Y axis is values of OD at 450 nm.

FIGS. 27A-27B show whole cell binding of Herceptin and Immu-132 to SKOV-3 cells (27A) & OE-19 cells (27B), as determined by flow cytometry with AlexaFluor488 conjugated anti-human IgG Fc as a secondary antibody. X axis is values of antibody concentration in nanomolar. Y axis is values of Median Fluorescence Intensity of AlexaFluor 488.

FIG. 28A-28B show the effects of vc-MMAE conjugated anti-HER2 (11D5_MMAE & Herceptin_MMAE) or anti-TROP2 (1H11_MMAE & Immu-132_MMAE) monospecific antibodies or anti-HER2/TROP2BsAb (BsAb_MMAE) in killing of SKOV-3 cells (28A) & OE-19 cells (28B). The viable cells were measured by adding cell counting-8 (CCK-8) reagent and reading at OD 450 nm. X axis is values of antibody concentration in nanomolar. Y axis is values of OD at 450 nm.

FIGS. 29A-29D show whole cell binding of Herceptin and Immu-132 to NCI-H1975 cells (29A), Colo205 cells (29B), T-47D cells (29C), & A431 (29D), as determined by flow cytometry with AlexaFluor488 conjugated anti-human IgG Fc as a secondary antibody. X axis is values of antibody concentration in nanomolar. Y axis is values of Median Fluorescence Intensity of AlexaFluor 488.

FIGS. 30A-30E show the effects of vc-MMAE conjugated anti-HER2 (11D5_MMAE & Herceptin_MMAE) or anti-TROP2 (1H11_MMAE & Immu-132_MMAE) monospecific antibodies or anti-HER2/TROP2 BsAb (BsAb_MMAE) in killing of NCI-H441 cells (30A), NCI-H1975 cells (30B), Colo205 cells (30C), T-47D cells (30D) & A431 cells (30E). The viable cells were measured by adding cell counting-8 (CCK-8) reagent and reading at OD 450 nm. X axis is values of antibody concentration in nanomolar. Y axis is values of OD at 450 nm.

FIG. 31 shows the tumor volume data from the in vivo efficacy study for the A431 tumors in Nu/Nu nude mice. Treatment groups shown in the figure legends, with N=7 mice for each group. *P<0.01, **P<0.001 compared to the Negative Control by 2-way ANOVA Tukey's multiple comparisons analysis. X axis is the days of treatment. Y axis is the tumor volume.

FIG. 32 shows the tumor volume data from the in vivo efficacy study for the OE-19 tumors in the immunodeficient B-NDG mice. Treatment groups shown in the figure legends, with N=7 mice for each group. *P<0.05, ***P<0.0001 compared to the Negative Control by 2-way ANOVA Tukey's multiple comparisons analysis. X axis is the days of treatment. Y axis is the tumor volume.

FIG. 33 (SEQ ID NOs: 39, 23 and 24) shows CDR sequences of an exemplary anti-HER2 antibody described in the disclosure.

FIG. 34 (SEQ ID NOs: 25-33) shows CDR sequences of exemplary anti-TROP2 antibodies described in the disclosure.

FIG. 35 (SEQ ID NOs: 1-12) lists amino acid sequences of VHHs as described in the disclosure.

FIGS. 36A-36F show schematic diagrams of BsAb-ADCs with 2 Cysteines or 3 Cysteines in each hinge region in N-, C-terminal formats (36A & 36 B), or tandem formats (36C & 36D), or monospecific VHH_Fc-ADCs (36E & 36F).

### DETAILED DESCRIPTION

A bispecific antibody or antigen-binding fragment thereof is an artificial protein that can simultaneously bind to two different epitopes (e.g., on two different antigens). In some embodiments, a bispecific antibody or antigen-binding fragment thereof can have two arms (Arms A and B).

The present disclosure relates to anti-HER2 antibodies or antigen binding fragments thereof, anti-TROP2 antibodies or antigen binding fragments thereof, antigen-binding protein constructs (e.g., bispecific antibodies or antigen-binding fragments thereof) that specifically bind to two different antigens (e.g., HER2 and TROP2), and antibody drug conjugates.

### Heavy Chain Single Variable Domain (VHH) Antibodies

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain IgG. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain IgG is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, or nanobody) is the smallest antigen-binding domain generated by adaptive immune systems. The third Complementarity Determining Region (CDR3) of the variable region of these antibodies has been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into bispecific antibody constructs than constructs containing conventional VH-VL pairs or single domains based on VH domains.

### Anti-HER2 Antibodies and Antigen-Binding Fragments

Human epidermal growth factor receptor 2 (HER2) (also known as ERBB2) is a transmembrane receptor belonging to the epidermal growth factor receptor subfamily of receptor protein tyrosine kinases. HER2 is overexpressed in various cancer types such as breast cancer and gastric cancer and has been reported to be a negative prognostic factor in breast cancer.

HER2 is a member of the human epidermal growth factor receptor (HER/EGFR/ERBB) family. But contrary to other member of the ERBB family, HER2 does not directly bind ligand. HER2 activation results from heterodimerization with another ERBB member or by homodimerization when HER2 concentration are high, for instance in cancer. Amplification or over-expression of this oncogene has been shown to play an important role in the development and progression of certain aggressive types of breast cancer. In recent years the protein has become an important biomarker and target of therapy for approximately 30% of breast cancer patients.

A detailed review of HER2 and its role in cancer can be found in Krishnamurti, Uma, and Jan F. Silverman. “HER2 in breast cancer: a review and update.” Advances in anatomic pathology 21.2 (2014): 100-107; Gutierrez, Carolina, and Rachel Schiff. “HER2: biology, detection, and clinical implications.” Archives of pathology & laboratory medicine 135.1 (2011): 55-62; Oh, Do-Youn, and Yung-Jue Bang. “HER2-targeted therapies-a role beyond breast cancer.” Nature Reviews Clinical Oncology 17.1 (2020): 33-48, each of which is incorporated herein by reference in its entirety.

The disclosure provides e.g., anti-HER2 antibodies, the modified antibodies thereof, the chimeric antibodies thereof, and the humanized antibodies thereof. The disclosure also provides VHH of these antibodies. These VHHs can be used in various multispecific antibody constructs as described herein. The disclosure also provides antigen-binding protein constructs containing an antigen binding region that is derived from these anti-HER2 antibodies.

The CDR sequences for 11D5, and 11D5 derived antibodies (e.g., humanized antibodies) include CDRs of the VHH domain as set forth in SEQ ID NOs: 22, 23, and 24, respectively. The amino acid sequence for the VHH domain of 11D5 antibody is set forth in SEQ ID NO: 1.

The amino acid sequences for various modified or humanized VHH are also provided. As there are different ways to modify or humanize a llama antibody (e.g., a sequence can be modified with different amino acid substitutions), the antibody can have more than one version of humanized sequences. In some embodiments, the humanized VHH domain is at least 80%, 85%, 90%, 91%, 92%, 93%, 94%, 95%, 96%, 97%, 98%, or 99% identical to the sequence of SEQ ID NO: 1.

Furthermore, in some embodiments, the antibodies or antigen-binding fragments thereof described herein can also contain one, two, or three VHH domain CDRs selected from the group of SEQ ID NOs: 22-24.

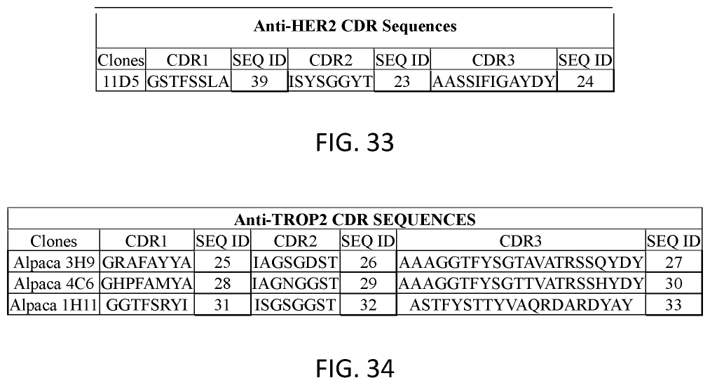

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 33.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 33.

In some embodiments, the antibody or an antigen-binding fragment described herein can contain a heavy-chain antibody variable domain (VHH) containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

The insertions, deletions, and substitutions can be within the CDR sequence, or at one or both terminal ends of the CDR sequence. In some embodiments, the CDR is determined based on IMGT definition.

In some embodiments, the antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is SEQ ID NO: 1.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

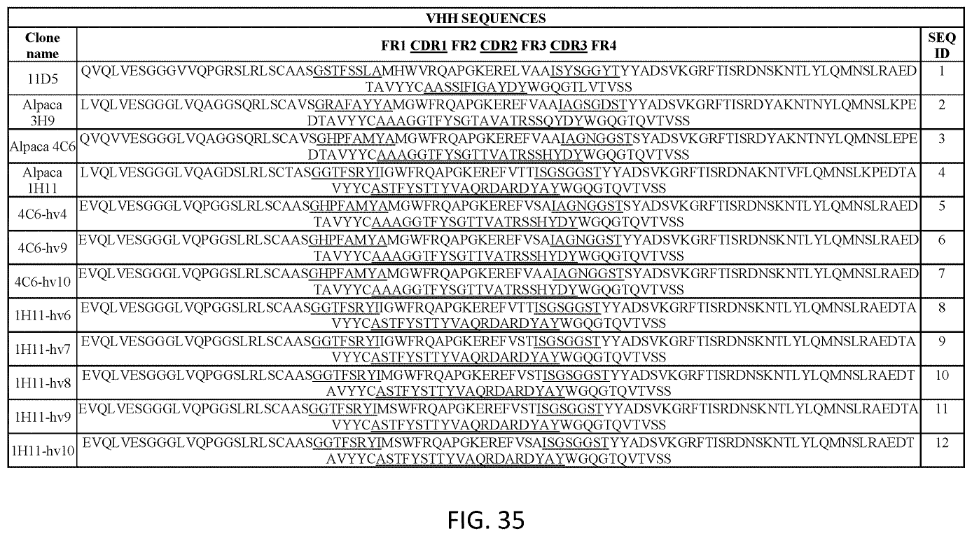

The disclosure also provides nucleic acid comprising a polynucleotide encoding a polypeptide comprising an immunoglobulin heavy-chain antibody variable domain (VHH). The VHH comprises CDRs as shown in FIG. 33, or has sequences as shown in FIG. 35.

In some embodiments, the antibody or antigen-binding fragment thereof comprises a heavy-chain antibody variable domain (VHH) CDR1 of SEQ ID NO: 22. In some embodiments, the antibody or antigen-binding fragment thereof comprises a heavy-chain antibody variable domain (VHH) CDR2 of SEQ ID NO: 23. In some embodiments, the antibody or antigen-binding fragment thereof comprises a heavy-chain antibody variable domain (VHH) CDR3 of SEQ ID NO: 24.

The anti-HER2 antibodies and antigen-binding fragments can also be antibody variants (including derivatives and conjugates) of antibodies or antibody fragments and multi-specific (e.g., bispecific) antibodies or antibody fragments. Additional antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgAQ1, and IgA2), or subclass. In some embodiments, the antibody or antigen-binding fragment thereof is an IgG antibody or antigen-binding fragment thereof.

### Anti-TROP2 Antibodies and Antigen-Binding Fragments

Trophoblast cell-surface antigen 2 (TROP2), also known as Tumor-associated calcium signal transducer 2 (TACSTD2), is a cell surface glycoprotein encoded and expressed by the TACSTD2 gene. It has high structural sequence similarity with epithelial adhesion molecule Epcam. TROP2 is a protein closely related to tumors. It mainly promotes tumor cell growth, proliferation and metastasis by regulating calcium ion signaling pathways, cyclin expression, and reducing fibronectin adhesion. Studies have found that TROP2 protein is highly expressed in breast cancer, colon cancer, bladder cancer, gastric cancer, oral squamous cell carcinoma, pancreatic cancer and ovarian cancer. The protein can promote tumor cell proliferation, invasion, metastasis, spread and other processes. In addition, in breast cancer and other cancers, the high expression of TROP2 has also been found to be closely related to more aggressive diseases and poor clinical prognosis of tumors.

TROP2 is an intracellular calcium signal transducer that is differentially expressed in many cancers. It signals cells for self-renewal, proliferation, invasion, and survival. It has stem cell-like qualities. TROP2 is expressed in many normal tissues, though in contrast, it is overexpressed in many cancers and the overexpression of TROP2 is of prognostic significance. Several ligands have been proposed that interact with TROP2. TROP2 signals the cells via different pathways and it is transcriptionally regulated by a complex network of several transcription factors. TROP2 expression in cancer cells has been correlated with drug resistance.

A detailed review of TROP2 and its overexpression in cancers can be found in Shvartsur, Anna, and Benjamin Bonavida. “TROP2 and its overexpression in cancers: regulation and clinical/therapeutic implications.” Genes & cancer 6.3-4 (2015): 84, which is incorporated herein by reference in its entirety.

The disclosure provides e.g., anti-TROP2 antibodies, the modified antibodies thereof, the chimeric antibodies thereof, and the humanized antibodies thereof. The disclosure also provides VHH of these antibodies. These VHHs can be used in various multispecific antibody constructs as described herein. The disclosure also provides antigen-binding protein constructs containing an antigen binding region that is derived from these anti-TROP2 antibodies.

The CDR sequences for Alpaca 3H9 (3H9), and 3H9 derived antibodies (e.g., humanized antibodies) include CDRs of the VHH domain as set forth in SEQ ID NOs: 25, 26, and 27, respectively. The amino acid sequence for the VHH domain of 3H9 antibody is set forth in SEQ ID NO: 2.

The CDR sequences for Alpaca 4C6 (4C6), and 4C6 derived antibodies (e.g., humanized antibodies) include CDRs of the VHH domain as set forth in SEQ ID NOs: 28, 29, and 30, respectively. The amino acid sequence for the VHH domain of 4C6 antibody is set forth in SEQ ID NO: 3.

The CDR sequences for Alpaca 1H11 (1H11), and 1H11 derived antibodies (e.g., humanized antibodies) include CDRs of the VHH domain as set forth in SEQ ID NOs: 31, 32, and 33, respectively. The amino acid sequence for the VHH domain of 1H11 antibody is set forth in SEQ ID NO: 4.

The amino acid sequences for various modified or humanized VHH are also provided. As there are different ways to modify or humanize a llama or alpaca antibody (e.g., a sequence can be modified with different amino acid substitutions), the antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOs: 2-12.

Furthermore, in some embodiments, the antibodies or antigen-binding fragments thereof described herein can also contain one, two, or three VHH domain CDRs selected from the group of SEQ ID NOs: 25-27, SEQ ID NOs: 28-30, and SEQ ID NOs: 31-33.

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 34.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 34.

In some embodiments, the antibody or an antigen-binding fragment described herein can contain a heavy-chain antibody variable domain (VHH) containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

The insertions, deletions, and substitutions can be within the CDR sequence, or at one or both terminal ends of the CDR sequence. In some embodiments, the CDR is determined based on IMGT definition.

In some embodiments, the antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is SEQ ID NO: 2. In some embodiments, the selected VHH sequence is SEQ ID NO: 3. In some embodiments, the selected VHH sequence is SEQ ID NO: 4. In some embodiments, the selected VHH sequence is SEQ ID NO: 5. In some embodiments, the selected VHH sequence is SEQ ID NO: 6. In some embodiments, the selected VHH sequence is SEQ ID NO: 7. In some embodiments, the selected VHH sequence is SEQ ID NO: 8. In some embodiments, the selected VHH sequence is SEQ ID NO: 9. In some embodiments, the selected VHH sequence is SEQ ID NO: 10. In some embodiments, the selected VHH sequence is SEQ ID NO: 11. In some embodiments, the selected VHH sequence is SEQ ID NO: 12.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy-chain antibody variable domain (VHH). The VHH comprises CDRs as shown in FIG. 34, or has sequences as shown in FIG. 35.

The anti-TROP2 antibodies and antigen-binding fragments can also be antibody variants (including derivatives and conjugates) of antibodies or antibody fragments and multi-specific (e.g., bispecific) antibodies or antibody fragments. Additional antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgAQ1, and IgA2), or subclass. In some embodiments, the antibody or antigen-binding fragment thereof is an IgG antibody or antigen-binding fragment thereof.

### Anti-HER2/TROP2 Bispecific Antibodies

The anti-HER2, anti-TROP2, or anti-HER2/TROP2 antigen-binding protein construct (e.g., antibodies, bispecific antibodies, trispecific antibodies, multi-specific antibodies, or antibody fragments thereof) can include an antigen binding site that is derived from any anti-HER2 antibody, anti-TROP2 antibody, or any antigen-binding fragment thereof as described herein.

In some embodiments, the bispecific antibodies are designed to include a VHH that targets HER2 and a VHH that targets TROP2. In some embodiments, the present disclosure provides bispecific antibodies that bind to both HER2 and TROP2. The bispecific antibodies can be used to treat HER2 or TROP2 positive cancers (e.g., non-small cell lung cancer) in a subject.

The HER2/TROP2 bispecific antibodies with specific structures are described below.

### BiSpecific-VI Structure

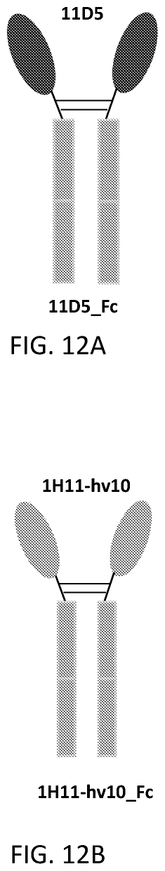

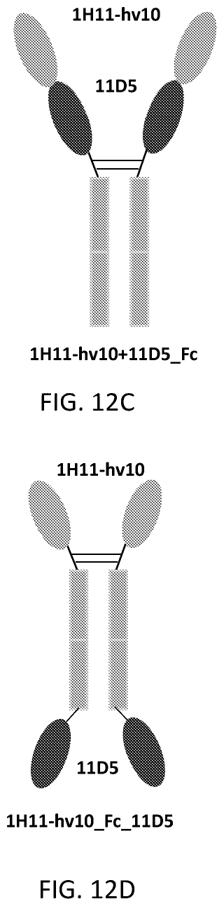

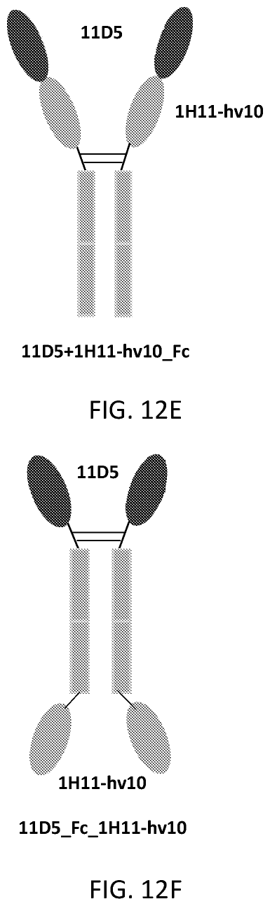

As shown in FIG. 12F, a HER2/TROP2 bispecific antibody can be prepared to have a BiSpecific-V1 structure. Specifically, the HER2/TROP2 bispecific antibody comprises (a) a first polypeptide comprising from N-terminus to C-terminus: a first heavy-chain antibody variable domain (VHH1), a first hinge region, a first CH2, a first CH3, and a third heavy-chain antibody variable domain (VHH2); and (b) a second polypeptide comprising from N-terminus to C-terminus: a second heavy-chain antibody variable region (VHH3), a second hinge region, a second CH2, a second CH3, and a fourth heavy-chain antibody variable region (VHH4).

In some embodiments, the VHH1 and the VHH3 specifically bind to HER2. In some embodiments, the VHH2 and the VHH4 specifically bind to TROP2. In some embodiments, sequences of the VHH1 and the VHH3 are identical. In some embodiments, sequences of the VHH2 and the VHH4 are identical.

In some embodiments, the HER2/TROP2 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 18. In some embodiments, the second polypeptide comprises a sequence that is at least 80%, 85%, 90%, 95%, or 100% identical to SEQ ID NO: 18.

In some embodiments, the first polypeptide comprises a sequence that is at least 80%, 85%, 90%, 95%, or 100% identical to SEQ ID NO: 1, SEQ ID NO: 36 or 37, and SEQ ID NO: 12. In some embodiments, the second polypeptide comprises a sequence that is at least 80%, 85%, 90%, 95%, or 100% identical to SEQ ID NO: 1, SEQ ID NO: 36 or 37, and SEQ ID NO: 12.

In some embodiments, the first CH2 and first CH3 and/or the second CH2 and second CH3 comprises an aspartic acid (Asp) at position 239 according to EU numbering. In some embodiments, the first CH2 and first CH3 and/or the second CH2 and second CH3 comprises a glutamic acid (Glu) at position 332 according to EU numbering.

In some embodiments, the VHH2 is linked to the C-terminus of the first CH2 and first CH3 via a first linker peptide sequence. In some embodiments, the VHH4 is linked to the C-terminus of the second CH2 and second CH3 via a second linker peptide sequence.

In some embodiments, the first and/or second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 38). In some embodiments, the first and/or second linker peptide sequence comprises a sequence that selected from GSGGSGGSGGSG (SEQ ID NO: 34) and GSGGSGGSGGSGGSG (SEQ ID NO: 35).

### BiSpecific-V2 Structure

As shown in FIG. 12E, a HER2/TROP2 bispecific antibody can be prepared to have a BiSpecific-V2 structure. Specifically, the HER2/TROP2 bispecific antibody comprises (a) a first polypeptide comprising from N-terminus to C-terminus: a VHH1, a VHH2, a first hinge region, a first CH2, and a first CH3; and (b) a second polypeptide comprising from N-terminus to C-terminus: a VHH3, a VHH4, a second hinge region, a second CH2, and a second CH3.

In some embodiments, the VHH1 and the VHH3 specifically bind to HER2. In some embodiments, the VHH2 and VHH4 specifically bind to TROP2. In some embodiments, sequences of the VHH1 and the VHH3 are identical. In some embodiments, sequences of the VHH2 and the VHH4 are identical.

In some embodiments, the HER2/TROP2 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 17. In some embodiments, the second polypeptide comprises a sequence that is at least 80%, 85%, 90%, 95%, or 100% identical to SEQ ID NO: 17.

In some embodiments, the first polypeptide comprises a sequence that is at least 80%, 85%, 90%, 95%, or 100% identical to SEQ ID NO: 1, SEQ ID NO: 34 or 35, SEQ ID NO: 12, and SEQ ID NO: 36 or 37. In some embodiments, the second polypeptide comprises a sequence that is at least 80%, 85%, 90%, 95%, or 100% identical to SEQ ID NO: 1, SEQ ID NO: 34 or 35, SEQ ID NO: 12, and SEQ ID NO: 36 or 37.

In some embodiments, the first CH2 and first CH3 and/or the second CH2 and second CH3 comprise an aspartic acid (Asp) at position 239 according to EU numbering. In some embodiments, the first CH2 and first CH3 and/or the second CH2 and second CH3 comprise a glutamic acid (Glu) at position 332 according to EU numbering.

In some embodiments, the VHH1 is linked to the N-terminus of the VHH2 via a first linker peptide sequence. In some embodiments, the VHH3 is linked to the N-terminus of the VHH4 via a second linker peptide sequence.

In some embodiments, the first and/or second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 38). In some embodiments, the first and/or second linker peptide sequence comprises a sequence that selected from GSGGSGGSGGSG (SEQ ID NO: 34), and GSGGSGGSGGSGGSG (SEQ ID NO: 35).

### BiSpecific-V3 Structure

As shown in FIG. 12C, a HER2/TROP2 bispecific antibody can be prepared to have a BiSpecific-V3 structure. Specifically, the HER2/TROP2 bispecific antibody comprises (a) a first polypeptide comprising from N-terminus to C-terminus: a VHH2, a VHH1, a first hinge region, and a first CH2, and a first CH3; and (b) a second polypeptide comprising from N-terminus to C-terminus: a VHH4, a VHH3, a second hinge region, a second CH2, and a second CH3.

In some embodiments, the VHH1 and the VHH3 specifically bind to HER2. In some embodiments, the VHH2 and the VHH4 specifically bind to TROP2. In some embodiments, sequences of the VHH1 and the VHH3 are identical. In some embodiments, sequences of the VHH2 and the VHH4 are identical.

In some embodiments, the first polypeptide comprises a sequence that is at least 80%, 85%, 90%, 95%, or 100% identical to SEQ ID NO: 15 or 21. In some embodiments, the second polypeptide comprises a sequence that is at least 80%, 85%, 90%, 95%, or 100% identical to SEQ ID NO: 15 or 21.

In some embodiments, the first polypeptide comprises a sequence that is at least 80%, 85%, 90%, 95%, or 100% identical to SEQ ID NO: 12, SEQ ID NO: 34 or 35, SEQ ID NO: 1, and SEQ ID NO: 36 or 37. In some embodiments, the second polypeptide comprises a sequence that is at least 80%, 85%, 90%, 95%, or 100% identical to SEQ ID NO: 12, SEQ ID NO: 34 or 35, SEQ ID NO: 1, and SEQ ID NO: 36 or 37.

In some embodiments, the first CH2 and first CH3 and/or the second CH2 and second CH3 comprise an aspartic acid (Asp) at position 239 according to EU numbering. In some embodiments, the first CH2 and first CH3 and/or the second CH2 and second CH3 comprise a glutamic acid (Glu) at position 332 according to EU numbering.

In some embodiments, the VHH2 is linked to the N-terminus of the VHH1 via a first linker peptide sequence. In some embodiments, the VHH4 is linked to the N-terminus of the VHH3 via a second linker peptide sequence.

In some embodiments, the first and/or second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 38). In some embodiments, the first and/or second linker peptide sequence comprises a sequence that selected from GSGGSGGSGGSG (SEQ ID NO: 34), and GSGGSGGSGGSGGSG (SEQ ID NO: 35).

### BiSpecific-V4 Structure

As shown in FIG. 12D, a HER2/TROP2 bispecific antibody can be prepared to have a BiSpecific-V4 structure. Specifically, the HER2/TROP2 bispecific antibody comprises (a) a first polypeptide comprising from N-terminus to C-terminus: a VHH2, a first hinge region, a first CH2, a first CH3, and a VHH1; and (b) a second polypeptide comprising from N-terminus to C-terminus: a VHH4, a second hinge region, a second CH2, a second CH3, and a VHH3.

In some embodiments, the VHH1 and the VHH3 specifically bind to HER2. In some embodiments, the VHH2 and the VHH4 specifically bind to TROP2. In some embodiments, sequences of the VHH1 and the VHH3 are identical. In some embodiments, sequences of the VHH2 and the VHH4 are identical.

In some embodiments, the HER2/TROP2 bispecific antibody comprises knob-into-hole mutations. In some embodiments, the Fc region is an IgG1 Fc region. In some embodiments, the first polypeptide comprises a sequence that is at least 80%, 85%, 90%, 95%, or 100% identical to SEQ ID NO: 16 or 20. In some embodiments, the second polypeptide comprises a sequence that is at least 80%, 85%, 90%, 95%, or 100% identical to SEQ ID NO: 16 or 20.

In some embodiments, the first polypeptide comprises a sequence that is at least 80%, 85%, 90%, 95%, or 100% identical to SEQ ID NO: 12, SEQ ID NO: 36 or 37, and SEQ ID NO: 1. In some embodiments, the second polypeptide comprises a sequence that is at least 80%, 85%, 90%, 95%, or 100% identical to SEQ ID NO: 12, SEQ ID NO: 36 or 37, and SEQ ID NO: 1.

In some embodiments, the first CH2 and first CH3 and/or the second CH2 and second CH3 comprise an aspartic acid (Asp) at position 239 according to EU numbering. In some embodiments, the first CH2 and first CH3 and/or the second CH2 and second CH3 comprise a glutamic acid (Glu) at position 332 according to EU numbering.

In some embodiments, the VHH1 is linked to the C-terminus of the first CH2 and first CH3 via a first linker peptide sequence. In some embodiments, the VHH3 is linked to the C-terminus of the second CH2 and second CH3 via a second linker peptide sequence.

In some embodiments, the first and/or second linker peptide sequence comprise a sequence that is at least 80%, 85%, 90%, 95%, or 100% identical to one or more (e.g., 1, 2, 3, 4, 5, 6, 7, or 8) repeats of GGGGS (SEQ ID NO: 38). In some embodiments, the first and/or second linker peptide sequence comprises a sequence that selected from GSGGSGGSGGSG (SEQ ID NO: 34) and GSGGSGGSGGSGGSG (SEQ ID NO: 35).

### Antibody and ADC Characteristics

The anti-HER2 antigen-binding protein construct (e.g., antibodies, bispecific antibodies, or antibody fragments thereof) or ADC derived therefrom can include an antigen-binding region that is derived from any anti-HER2 antibody or any antigen-binding fragment thereof as described herein.

In some embodiments, the antibodies, or antigen-binding fragments thereof described herein can bind to HER2 and/or TROP2, thereby blocking the interaction of these receptors and their respective ligands; decreasing the phosphorylation of downstream signaling pathways (e.g., ERK and/or Akt pathways); and/or directly killing the cancer cells by ADCC and/or CDC.

In some embodiments, the binding to HER2 can be determined by whole cell binding assays using cells that express HER2 (e.g., CHO-h-HER2 stable cells, N87 cells or SKBR3 cells). In some embodiments, the EC50 of the antibodies or antigen-binding fragments thereof described herein for binding to HER2 is less than or about 100 nM, less than or about 50 nM, less than or about 25 nM, less than or about 12.5 nM, less than or about 10 nM, less than or about 7.5 nM, less than or about 5 nM, less than or about 3 nM, less than or about 2.5 nM, less than or about 2.0 nM, less than or about 1.5 nM, less than or about 1.1 nM, less than or about 1 nM, less than or about 0.9 nM, less than or about 0.8 nM, less than or about 0.7 nM.

In some embodiments, the binding to different domains of HER2 extracellular domain (ECD) can be determined by ELISA binding assays using his-tagged HER2 domain fragments. In some embodiments, the antibodies, or antigen-binding fragments thereof described herein can bind to Domain I, Domain II, Domain III, and/or Domain IV of the HER2 ECD.

In some embodiments, the effects of the antibodies or antigen-binding fragments thereof described herein on PBMC-mediated killing of HER2 expressing breast cancer cells can be determined by in vitro cell killing assays using PBMCs and cancer cells expressing HER2 (e.g., AU-565 cells or SKBR3 cells). In some embodiments, the IC50 of the antibodies or antigen-binding fragments thereof described herein for inducing cell killing is less than or about 1 nM, less than or about 0.5 nM, less than or about 0.4 nM, less than or about 0.3 nM, less than or about 0.25 nM, less than or about 0.2 nM, or less than or about 0.15 nM.

In some embodiments, the effects of the antibodies or antigen-binding fragments thereof described herein on proliferation of cancer cells expressing HER2 (e.g., AU-565 cells or SKBR3 cells) can be determined using an in vitro proliferation assay. In some embodiments, the IC50 of the antibodies or antigen-binding fragments thereof described herein for inhibiting cell proliferation is less than or about 2 nM, less than or about 1.5 nM, less than or about 1.25 nM, less than or about 1 nM, less than or about 0.9 nM, or less than or about 0.85 nM.

In some embodiments, the binding to TROP2 (e.g., human TROP2 or mouse TROP2) can be determined by whole cell binding assays using cells that express TROP2 (e.g., CHO-h-TROP2 stable cells, 293T-m-TROP2 cells, SKBR3 cells, NCI-H1975 cells or Colo205 cells). In some embodiments, the EC50 of the antibodies or antigen-binding fragments thereof described herein for binding to TROP2 is less than or about 1250 nM, less than or about 1000 nM, less than or about 750 nM, less than or about 500 nM, less than or about 250 nM, less than or about 100 nM, less than or about 75 nM, less than or about 50 nM, less than or about 25 nM, less than or about 12.5 nM, less than or about 10 nM, less than or about 7.5 nM, less than or about 5 nM, is less than or about 3 nM, less than or about 2.5 nM, less than or about 2 nM, less than or about 1.75 nM, less than or about 1.5 nM, less than or about 1.25 nM, or less than or about 1 nM.

In some embodiments, the binding to different domains of TROP2 can be determined by competition binding assays. In some embodiments, the antibodies, or antigen-binding fragments thereof described herein can bind to the same binding domain of TROP2. In some embodiments, the antibodies, or antigen-binding fragments thereof described herein can bind to different binding domains of TROP2.

Thermal stabilities can also be determined. The antibodies, the antigen-binding fragments thereof, or the antigen-binding protein constructs (e.g., bispecific antibody), or the ADC derived therefrom as described herein can have a Tm greater than 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies, the antigen-binding fragments thereof, the antigen-binding protein constructs (e.g., the anti-TROP2 antibody, the anti-HER2 antibody, or the bispecific antibody), or the ADC derived therefrom, has a purity that is greater than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by SEC-HPLC. In some embodiments, the antibodies, the purity is less than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by SEC-HPLC.

General techniques for measuring the affinity of an antibody for an antigen include, e.g., ELISA, RIA, and surface plasmon resonance (SPR).

In some embodiments, the binding to HER2 and TROP2 can be determined by whole cell binding assays using cells that express HER2 and TROP2 (e.g., SKBR3 cells, NCI-H441 cells, or HCC202 cells). In some embodiments, the EC50 of the antibodies or antigen-binding fragments thereof described herein for binding to HER2 is less than or about 125 nM, less than or about 100 nM, less than or about 50 nM, less than or about 25 nM, less than or about 12.5 nM, less than or about 10 nM, less than or about 7.5 nM, less than or about 5 nM, less than or about 3 nM, less than or about 2.5 nM, less than or about 2.0 nM, less than or about 1.5 nM, less than or about 1.1 nM, less than or about 1 nM, less than or about 0.9 nM, less than or about 0.8 nM, less than or about 0.7 nM.

In some embodiments, the effects of the antibodies, the antigen-binding fragments thereof, or the antigen-binding protein constructs (e.g., bispecific antibody), on cell killing can be determined using a cell killing assay using a 2^{nd} Ab-MMAE. In some embodiments, the IC50 of the antibodies or antigen-binding fragments thereof described herein for inducing cell killing is less than or about 1 nM, less than or about 0.5 nM, less than or about 0.4 nM, less than or about 0.3 nM, less than or about 0.25 nM, less than or about 0.2 nM, less than or about 0.15 nM, less than or about 0.125 nM, or less than or about 0.1 nM.

In some embodiments, the drug antibody ratio (DAR) in the ADC described herein can be determined by hydrophobic interaction chromatography (HIC-HPLC). In some embodiments, the majority DAR species is D4. In some embodiments, D4 constitutes more than 70%, more than 75%, more than 80%, more than 85%, more than 90%, more than 95%, more than 96%, more than 97%, more than 98%, or more than 99% of the DAR species. In some embodiments, the DAR in the ADC described herein is higher than 3, higher than 3.1, higher than 3.2, higher than 3.3, higher than 3.4, higher than 3.5, higher than 3.6 higher than 3.7 higher than 3.8, or higher than higher than 3.9. In some embodiments, the DAR in the ADC described herein is lower than 4.1, lower than 4.2, lower than 4.3, lower than 4.4, lower than 4.5, or lower than 4.6. In some embodiments, the DAR in the ADC described herein is about 3.7-4, about 3.8-4.1, about 3.9-4.2, about 3.9-4.1, or about 4.

In some embodiments, the effects of the ADC described herein on cell killing can be determined using a cell killing assay using cancer cells (e.g., NCI-H441 cells, AU-565 cells, SKBR3 cells, N87 cells, SK-OV3 cells, OE-19 cells, NCI-H1975 cells, Colo205 cells, or T-47D cells). In some embodiments, the IC50 of the antibodies or antigen-binding fragments thereof described herein for inducing cell killing is less than or about 100 nM, less than or about 50 nM, less than or about 25 nM, less than or about 10 nM, less than or about 5 nM, less than or about 1 nM, less than or about 0.5 nM, less than or about 0.4 nM, less than or about 0.3 nM, less than or about 0.25 nM, less than or about 0.2 nM, less than or about 0.15 nM, less than or about 0.125 nM, less than or about 0.1 nM, less than or about 0.05 nM, less than or about 0.04 nM, less than or about 0.03 nM, or less than or about 0.02 nM.

In some embodiments, the antibody, the antigen-binding fragment thereof, or the antigen-binding protein construct (e.g., bispecific antibody) has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4. In some embodiments, one or both mutations S239D and/or I332E (SI mutations) are introduced in antibody Fc region to enhance the antibody affinity to FcγRIIIA, thereby increasing ADCC effects. A detailed description of SI mutations can be found in U.S. Pat. No. 7,662,925, which is incorporated by reference in their entirety.

### Antibodies and Antigen Binding Fragments

The present disclosure provides antibodies and antigen-binding fragments thereof that comprise complementary determining regions (CDRs), VHHs, heavy chain variable regions, light chain variable regions, heavy chains, or light chains described herein.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. The IMGT numbering for V-DOMAIN (IG and TR) is derived from the IMGT unique numbering for V-REGION. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); Kontermann, R., & Dubel, S. (Eds.). (2010). Antibody engineering: Volume 2. Springer; Lefranc M.-P., “The IMGT unique numbering for Immunoglobulins, T cell receptors and Ig-like domains” The Immunologist, 7, 132-136 (1999); each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, VHH, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv), or VHH as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies or antigen-binding fragments thereof can bind to two different antigens or two different epitopes. In some embodiments, the antibodies or antigen-binding fragments thereof can bind to three different antigens or three different epitopes.

An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

In some embodiments, the scFv described herein comprises from N-terminus to C-terminus: VH; the polypeptide linker; and VL. In some embodiments, the scFv described herein comprises from N-terminus to C-terminus: VL; the polypeptide linker; and VH.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Antibodies and antibody fragments of the present disclosure can be modified in the Fc region to provide desired effector functions or serum half-life. In some embodiments, the Fc region in any one of the antibody or antigen-binding fragment described herein comprises an aspartic acid (Asp) at position 239 according to EU numbering. In some embodiments, the Fc region in any one of the antibody or antigen-binding fragment described herein comprises a glutamic acid (Glu) at position 332 according to EU numbering. In some embodiments, the Fc region described herein is any one of the Fc regions described herein, comprising an aspartic acid (Asp) at position 239 according to EU numbering, and/or a glutamic acid (Glu) at position 332 according to EU numbering. In some embodiments, the Asp239 and/or Glu332 described herein can increase effector functions (e.g., ADCC or CDC) of an antibody or antigen binding fragment thereof by at least or about 10%, 20%, 30%, 40%, 50%, 60%, 70%, 80%, 90%, 1-fold, 2-fold, 5-fold, 10-fold, 20-fold, 50-fold, or 100-fold, as compared to those of a wild-type antibody or antigen-binding fragment thereof. Details can be found, e.g., in Lazar, G. A. et al., “Engineered antibody Fc variants with enhanced effector function.” Proceedings of the National Academy of Sciences 103.11 (2006): 4005-4010, which is incorporated herein by reference in its entirety.

In some embodiments, the Fc region in any one of the antibody or antigen-binding fragment described herein comprises a wild-type human IgG1 CH2 domain. In some embodiments, the Fc region in any one of the antibody or antigen-binding fragment described herein comprises a mutated human IgG1 CH2 domain.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multi-specific antibody or antigen-binding fragment thereof described herein (e.g., anti-HER2/TROP2 bispecific antibody) binds to an antigen (e.g., HER2) with a binding ability that is about 50%, about 60%, about 70%, about 80%, about 90%, about 100%, about 110%, about 120%, about 130%, about 140%, about 150%, or about 200% to that of a heavy-chain antibody (e.g., an anti-HER2 heavy-chain antibody) comprising the same VHH of the multi-specific antibody.

In some embodiments, the multi-specific antibody or antigen-binding fragment thereof described herein (e.g., a HER2/TROP2 bispecific antibody) binds to an antigen (e.g., TROP2) with a binding ability that is about 50%, about 60%, about 70%, about 80%, about 90%, about 100%, about 110%, about 120%, about 130%, about 140%, about 150%, or about 200% to that of an antibody or antigen-binding fragment (e.g., an anti-TROP2 heavy-chain antibody ) comprising the same VHH targeting TROP2 of the multi-specific antibody.

In some embodiments, the bispecific antibody or antigen-binding fragment thereof described herein (e.g., a HER2/TROP2 bispecific antibody) mediates complement-dependent cytotoxicity (CDC) or ADC to at least or about 1 fold, 2 folds, 3 folds, 4 folds, 5 folds, 6 folds, 7 folds, 8 folds, 9 folds, 10 folds, 11 folds, 12 folds, 13 folds, 14 folds, 15 folds, 16 folds, 17 folds, 18 folds, 19 folds, 20 folds, 30 folds, 40 folds, or 50 folds as compared to that mediated by an isotype control antibody.

### Antibody Drug Conjugates (ADC)

The antibodies, the antigen-binding fragments thereof, or the antigen-binding protein constructs (e.g., bispecific antibodies) described herein can be conjugated to a therapeutic agent (a drug). The therapeutic agent can be covalently or non-covalently bind to the antibody or antigen-binding fragment or the antigen binding protein construct (e.g., a bispecific antibody). In some embodiments, the bispecific antibody is an anti-HER2/TROP2 bispecific antibody.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

In some embodiments, the antigen-binding construct is coupled to the drug via a cleavable linker e.g. a SPBD linker or a maleimidocaproyl-valine-citrulline-p-aminobenzyloxycarbonyl (VC) linker. In some embodiments, the antigen-binding construct is coupled to the drug via a non-cleavable linker e.g. a MCC linker formed using SMCC or sulfo-SMCC. Selection of an appropriate linker for a given ADC can be readily made by the skilled person having knowledge of the art and taking into account relevant factors, such as the site of attachment to the antigen binding construct, any structural constraints of the drug and the hydrophobicity of the drug (see, for example, review in Nolting, Chapter 5, Antibody-Drug Conjugates: Methods in Molecular Biology, 2013, Ducry (Ed.), Springer). A number of specific linker-toxin combinations have been described and may be used with the antigen binding constructs described herein to prepare ADCs in certain embodiments. Examples include, but are not limited to, cleavable peptide-based linkers with auristatins such as MMAE and MMAF, camptothecins such as SN-38, duocarmycins and PBD dimers; non-cleavable MC-based linkers with auristatins MMAF and MMAE; acid-labile hydrazone-based linkers with calicheamicins and doxorubicin; disulfide-based linkers with maytansinoids such as DM1 and DM4, and bis-maleimido-trioxyethylene glycol (BMPEO)-based linkers with maytansinoid DM1. Some these therapeutic agents and linkers are described, e.g., in Peters & Brown, (2015) Biosci. Rep. e00225; Dosio et al., (2014) Recent Patents on Anti-Cancer Drug Discovery 9:35-65; US Patent Publication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the antibodies through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the ADCs described here. Various prepared linkers, linker components and toxins are commercially available or may be prepared using standard synthetic organic chemistry techniques. These methods are described e.g., in March's Advanced Organic Chemistry (Smith & March, 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected antigen binding construct are also available commercially, for example, linker-toxins comprising DM1, DM4, MMAE, MMAF or Duocarmycin SA are available from Creative BioLabs (Shirley, N.Y.).

Several specific examples of methods of preparing ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p:5, may cause aggregation, insolubility, toxicity, or loss of cellular permeability of certain antibody-drug conjugates. In certain embodiments, the average drug loading for an antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the DAR is about or at least 1, 2, 3, 4, 5, 6, 7, or 8. In some embodiments, the average DAR in the composition is about 1˜ about 2, about 2˜ about 3, about 3˜ about 4, about 4˜ about 5, about 5˜ about 6, about 6˜ about 7, or about 7˜ about 8.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 1, 1%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

### Methods of Making Antibodies

An isolated fragment of human protein (e.g., HER2, TROP2, or cancer antigens) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDR1, 2 and 3, generated by overlapping PCR, to a modified human VH scaffold to generate enhanced diversity and keep low immunogenicity. The VHH libraries can be then panned against antigens to obtain VHH with desired binding ability.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding ability for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels, alpaca and llamas), chicken, goats, and rodents (e.g., rats, mice, hamsters and rabbits), including transgenic rodents genetically engineered to produce human antibodies.

Phage display (panning) can be used to optimize antibody sequences with desired binding abilities. In this technique, a gene encoding single chain Fv (comprising VH or VL) or VHH can be inserted into a phage coat protein gene, causing the phage to “display” the scFv or VHH on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding abilities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the hinge region of the IgG are substituted. In some embodiments, each hinge region has a C220S mutation according to EU numbering. In some embodiments, each hinge region has a Cysteine at position 220, a Cysteine at position 226 and a Cysteine at position 229, according to EU numbering. In some embodiments, each hinge region has a Serine at position 220, a Cysteine at position 226 and a Cysteine at position 229. In some embodiments, a payload (e.g., a drug) is covalently linked to each of the Cysteine at position 220, the Cysteine at position 226 and the Cysteine at position 229. In some embodiments, a payload (e.g., a drug) is covalently linked to each of the Cysteine at position 226 and the Cysteine at position 229. In some embodiments, the DAR of the antibody-drug conjugate is 4. In some embodiments, the DAR of the antibody-drug conjugate is 6.

In some embodiments, one or more amino acid residues in the CH3 portion of the IgG are substituted. In some embodiments, one heavy chain has one or more of the following substitutions Y349C and T366W. The other heavy chain can have one or more the following substitutions E356C, T366S, L368A, and Y407V. Furthermore, a substitution (-ppcpScp-->-ppcpPcp-) can also be introduced at the hinge regions of both substituted IgG. In some embodiments, one heavy chain has a T366Y (knob) substitution, and the other heavy chain has a Y407T (hole) substitution (EU numbering).

One aspect of the present application provides a heteromultimeric (e.g., heterodimeric) protein comprising a first polypeptide comprising a first heavy chain constant domain 3 (CH3) domain and a second polypeptide comprising a second CH3 domain, wherein the first CH3 domain comprises a substitution relative to a wildtype CH3 domain at amino acid position 354 with a bulky hydrophobic amino acid, and/or the second CH3 domain comprises a substitution relative to a wildtype CH3 domain at amino acid position 347 with a negatively charged amino acid, and wherein the amino acid residue numbering is based on EU numbering. In some embodiments, the bulky hydrophobic amino acid at amino acid position 354 forms a hydrophobic interaction with an amino acid residue in the second CH3 domain. In some embodiments, the second CH3 domain comprises a bulky hydrophobic residue at amino acid position 349 (e.g., Y349). In some embodiments, the negatively charged amino acid at amino acid position 347 forms an ionic bond with an amino acid residue in the first CH3 domain. In some embodiments, the first CH3 domain comprises a positively charged residue at amino acid position 360 (e.g., K360). In some embodiments, the first CH3 domain and the second CH3 domain are human CH3 domains. In some embodiments, the first CH3 domain comprises a substitution selected from the group consisting of S354Y, S354F and S354W. In some embodiments, the first CH3 domain comprises S354Y. In some embodiments, the second CH3 domain does not comprise a compensatory substitution (e.g., a substitution at Y349) for the substitution of S354 in the first CH3 domain. In some embodiments, the second CH3 domain comprises a substitution selected from the group consisting of Q347E and Q347D. In some embodiments, the second CH3 domain comprises Q347E. In some embodiments according to any one of the heteromultimeric proteins described above, the first CH3 domain and the second CH3 domain further comprise knob-into-hole (KIH) residues. In some embodiments, the knob-into-hole residues are T366Y and Y407T. In some embodiments, the first CH3 domain comprises T366Y and S354Y, and the second CH3 domain comprises Y407T and Q347E. In some embodiments, the first CH3 domain comprises Y407T and S354Y, and the second CH3 domain comprises T366Y and Q347E. Details can be found, e.g., in PCT/US2020/025469, which is incorporated herein by reference.

### Methods of Treatment

The methods described herein include methods for the treatment of disorders associated with cancer. Generally, the methods include administering a therapeutically effective amount of engineered antibodies, the antigen-binding fragments thereof, the antigen-binding protein constructs (e.g., bispecific antibodies), or the antibody drug conjugates as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of antibodies, the antigen-binding fragments thereof, the antigen-binding protein constructs (e.g., bispecific antibodies), or an antibody drug conjugate described herein to a subject in need thereof, e.g., a subject having, or identified or diagnosed as having, a cancer, e.g., breast cancer, carcinoid, cervical cancer, colorectal cancer, endometrial cancer, glioma, head and neck cancer, liver cancer, lung cancer, lymphoma, melanoma, ovarian cancer, pancreatic cancer, prostate cancer, renal cancer, skin cancer, stomach cancer, testis cancer, thyroid cancer, or urothelial cancer.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. In some embodiments, the subject is a human. In some embodiments, the subject is a dog.

In some embodiments, the cancer is thyroid cancer, urothelial cancer, breast cancer, colorectal cancer, renal cancer, cervical cancer, ovarian cancer, lung cancer, endometrial cancer, skin cancer, stomach cancer, pancreatic cancer, prostate cancer, liver cancer, lymphoma, or glioma.

In some embodiments, the cancer is cervical cancer, prostate cancer, thyroid cancer, urothelial cancer, head and neck cancer, endometrial cancer, ovarian cancer, lung cancer, breast cancer, carcinoid, skin cancer, liver cancer, or testis cancer.

In some embodiments, the cancer is pancreas cancer, lung cancer, stomach cancer, prostate cancer, breast cancer, ovary cancer, colon cancer, skin cancer, or brain cancer.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of the composition used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal.

A typical daily dosage of an effective amount of an antibody, the antigen-binding fragment thereof, the antigen-binding protein construct (e.g., a bispecific antibody) or the antibody drug conjugate is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, the antigen-binding fragment thereof, or the antigen-binding protein construct (e.g., a bispecific antibody), antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, the antigen-binding fragment thereof, the antigen-binding protein construct (e.g., a bispecific antibody), or antibody-drug conjugate, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, antibody-drug conjugate, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) or antibody-drug conjugate in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L1 antibody, an anti-LAG-3 antibody, an anti-TIGIT antibody, an anti-BTLA antibody, or an anti-GITR antibody.

### Pharmaceutical Compositions and Routes of Administration

Also provided herein are pharmaceutical compositions that contain at least one (e.g., one, two, three, or four) of the antigen-binding protein constructs, antibodies (e.g., bispecific antibodies), antigen-binding fragments, or antibody-drug conjugates described herein. Two or more (e.g., two, three, or four) of any of the antigen-binding protein constructs, antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antigen-binding protein constructs, antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Exemplary doses include milligram or microgram amounts of any of the antigen-binding protein constructs, antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 0.1 mg/kg to about 0.5 mg/kg).

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1: Anti-Human HER2 VHH Antibodies—Synthetic VHH Antibody Library Construction, Panning and Hits Identification

### Synthetic VHH Antibody Library Construction

Camelid/Alpaca VHH antibody is a type of heavy chain only antibody, which preserves both the binding and the functions of conventional antibodies with heavy chains (VH) and light chains (VL). The unique feature of VHH is that it has a long CDR3 compared to the VH CDR3 of conventional antibodies. A VHH synthetic library was designed. We used fixed lengths for CDR1 & CDR2, each containing 8 amino acids. For CDR3, we designed 13 different lengths from 10 to 22 amino acids. The designed VHH library was synthesized and cloned into a pADL-23c phagemid library. The phagemid VHH library was transformed to TG1 cells. 1.5 μg phagemid DNA produced>2.5×10⁹ TG1 colonies. In total, 12 transformations were made, and the combined diversity of TG1 VHH library is about 3×10¹⁰. Phage library was made thereafter by culturing 2 L of TG1 cells and 20 times helper phages. A standard protocol was used to purify the phage library. Final titer of phage library measured at OD260, with 2.37×10¹³/ml.

### Panning and Hits Identification

Antigens of recombinant human HER2 extracellular domain (h-HER2 ECD) was purchased from Sinobiological USA, and biotinylated with NHS-ester biotinylation reagent (EZ-Link Sulfo-NHS-SS-Biotin, ThermoFisher). Panning of binders to human HER2 antigen was performed using streptavidin-coupled Dynabeads coated with biotinylated h-HER2 ECD protein. After 3 rounds of panning, h-HER2 binders were eluted and used to infect SS320 cells. Colonies of SS320 cells were picked and cultured in 2YT medium. 0.5 mM IPTG was added to induce secretion of VHH antibodies. VHH antibodies in supernatants were screened by ELISA assays using 96-well plates coated with antigens of human HER2 Domain IV (Sinobiological, Pro489-Cys630 of human HER2), human HER2 Domain I (home-made, Met1-Cys195), or human HER2 Domain II+III+IV (home-made, Ser196-Cys630). In total, three, ten and fifteen 96-well plates of VHH supernatant samples (2660 clones) were screened for HER2 Domain IV, Domain I, and Domain II+III+IV antigens, respectively. Positive binders to different HER2 domains were further screened by flow cytometry whole cell binding assays using human HER2 stably transfected 293T cells. Sequencing was performed for cell surface h-HER2 binders. In total, 26 clones with different sequences were obtained. The CDR and framework sequences were obtained using IMGT (the international ImMunoGeneTics information system).

### Example 2: Characterization of HER2 VHH Antibodies

### Construction of Bivalent VHH-Fc Clones

26 unique clones were constructed to make bivalent antibodies with human IgG1 Fc region (VHH-Fc) by adding the hinge region and constant domains (CH2 and CH3). Constructed bivalent VHH-Fc antibodies were transiently expressed in ExpiCHO cells, and proteins in supernatants were harvested and purified by Protein A resin.

### Whole Cell Binding of Anti-HER2 VHH-Fc Clones to Human HER2

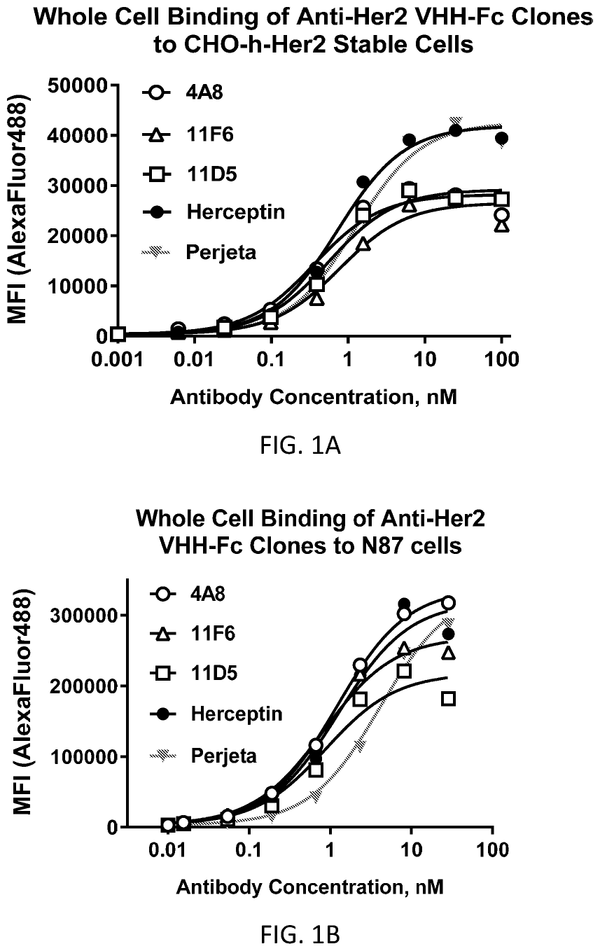

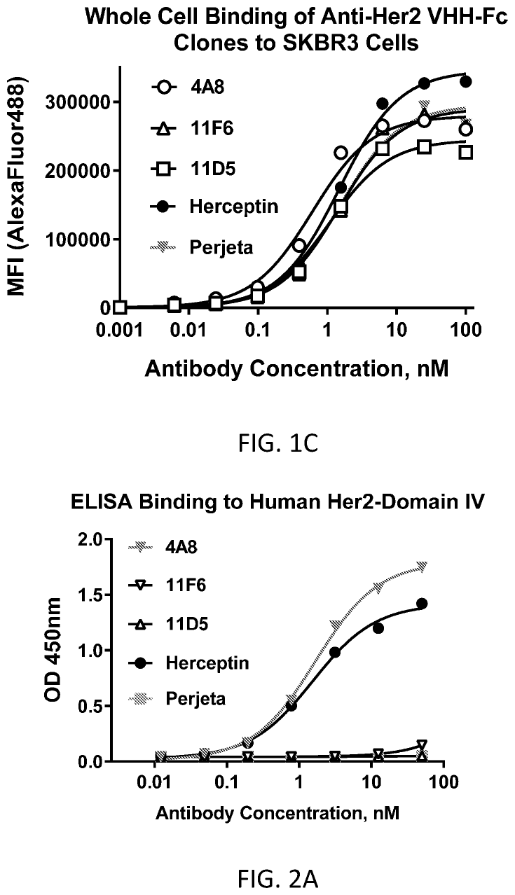

Binding of bivalent anti-HER2 VHH-Fc clones to human HER2 stably transfected CHO cells (CHO-h-HER2 stable cells) was determined by flow cytometry whole cell binding (WCB) assays. Briefly, antibodies were incubated at different concentrations with CHO-h-HER2 stable cells (0.2×10⁶/ml) in 100 μl/well in 96-well plates in FACS buffer (PBS with 1.0% FBS) for 30 min. After washing, Alexa Fluor 488 conjugated anti-human IgG Fc secondary antibody (Alexa Fluor® 488 AffiniPure Goat Anti-Human IgG, Fcγ fragment specific, Jackson labs, 1:750 dilution) was added, and incubated for 30 min. After washing, Median Fluorescence Intensity (MFI) was measured using CytoFlex (Beckman Coulter) by gating live cell population with FITC channel. Whole cell binding of VHH-Fc clones to endogenously expressed HER2 in human cancer cells was also determined using N87 (Stomach cancer cell line) and SKBR3 (Breast cancer cell line) cells, both of which are cancer cells with high HER2 expressions. EC50 of whole cell binding for 20 VHH-Fc clones to HER2 in CHO-h-HER2 stable cells and N87 cells, and their likely binding domain to HER2 (data from ELISA screening assay) are shown in the table below. Whole cell binding curves for top 3 VHH-Fc clones 4A8, 11F6 and 1 1D5, and reference antibodies Herceptin and Perjeta (from Roche) in CHO-h-HER2 stable cells, N87 cells & SKBR3 cells shown in FIGS. 1A, 1B & 1C.

TABLE 1
EC50 of whole cell binding to CHO-
h-HER2 stable cells & N87 cells

| | | | | Likely binding |
|---|---|---|---|---|
| | | CHO-h-HER2 | | domain of |
| # | Clones | stable cells | N87 cells | human HER2 |
| 1 | 4A8 | 0.71 | 0.91 | Domain IV |
| 2 | 1C10 | 1.61 | 6.09 | Domain IV |
| 3 | 1A10 | 20.55 | 2.20 | Domain IV |
| 4 | 1H4 | 8.33 | 6.02 | Domain IV |
| 5 | 4A10 | 29.05 | 4.38 | Domain IV |
| 6 | 6F10 | 9.52 | 7.67 | Domain IV |
| 7 | 8A6 | 7.15 | 7.81 | Domain IV |
| 8 | 20A3 | 2.57 | 30.20 | Domain IV |
| 9 | 21B1 | 1.15 | 4.25 | Domain IV |
| 10 | 5F5 | 3.46 | 8.80 | Domain IV |
| 11 | 11F6 | 1.87 | 4.37 | Domain IV |
| 12 | 11D5 | 2.09 | 3.136 | Domain II/III |
| 13 | 13B8 | 10.02 | 2.51 | Domain II/III |
| 14 | 21C1 | 0.68 | 5.25 | Domain II/III |
| 15 | 21A1 | 0.67 | 7.30 | Domain II/III |
| 16 | 7G11 | 60.98 | 6.00 | Domain II/III |
| 17 | 20C2 | 1.45 | 63.70 | Domain I |
| 18 | 5D1 | 4.74 | 77.30 | Domain I |
| 19 | 5D12 | 5.60 | 40.05 | Domain I |
| 20 | 5D5 | 1.23 | 2.00 | Domain I |
| Reference | Herceptin | 0.71 | 1.31 | Domain IV |
| Reference | Perjeta | 3.69 | 4.15 | Domain II |

### Determine Binding Domains of Human HER2 for Lead Anti-HER2 VHHT Antibodies

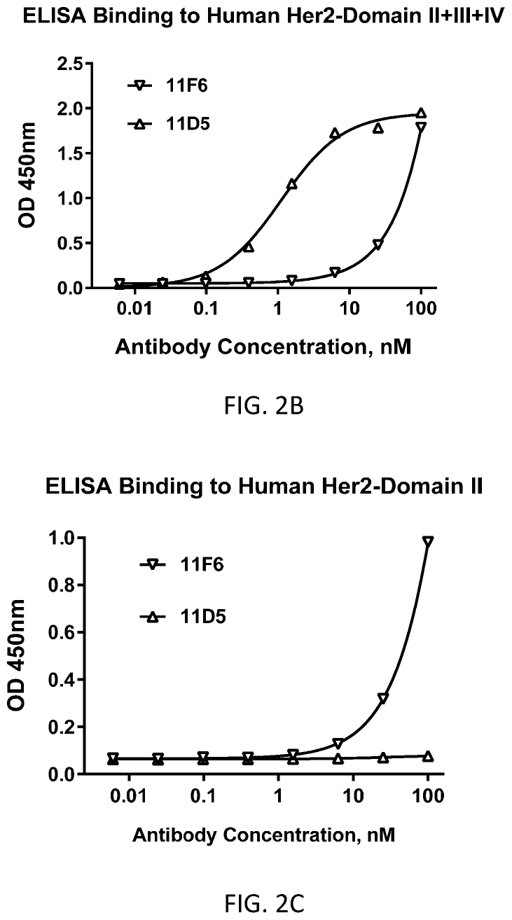

Binding of anti-HER2 VHH-Fc clones to different domains of human HER2 ECD was determined by ELISA binding assays using his-tagged HER2 domain fragments. Human HER2 domain IV fragment (Pro489-Cys630) was purchased from Sinobiological, domain II+III+IV fragment (Ser196-Cys630) & domain II fragment (Ser196-Asn319) were made. As shown in FIG. 2A, clone 4A8 and reference antibody Herceptin (but not Perjeta) bind to Domain IV. 11F6 and 11D5 do not bind to domain IV, indicating they bind to other domains. FIG. 2B shows that 11F6 (but not 111D5) binds to domain II, while both 11F6 and 11D5 bind to domain II+III+IV (FIG. 2C), suggesting that 11D5 binds to domain III of human HER2.

### Effects of Lead Anti-HER2 VHH-Fc Clones in PBMC-Mediated Killing of HER2 Expressing Breast Cancer Cells

One of the anti-tumor functions of anti-HER2 antibodies in vivo is antibody-dependent cell mediated cytotoxicity (ADCC), which involves binding of the antibody to tumor cells by the variable binding domain (e.g. Fab or VHH), and binding of the Fc domain to the Fcγ receptor (e.g. FcγRIII or CD16) on NK cells. The bridging between tumor cells and NK cells, leads to activation of NK cells, which then release proteins such as perforin and proteases to induce lysis of antibody bound tumor cells.

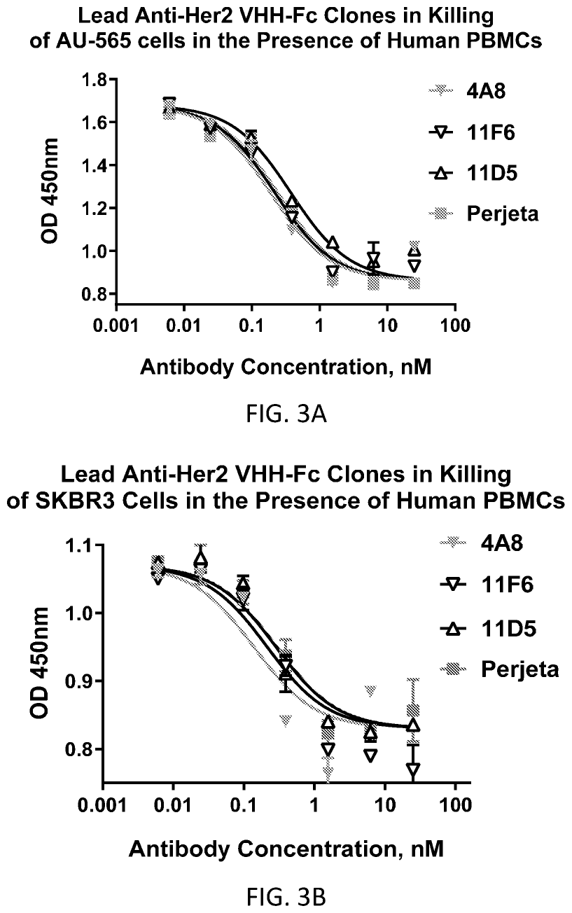

Human PBMCs (peripheral blood mononuclear cells, which contain NK cells) from normal donors were used. Briefly, cancer cells were seeded in 96-well plates at 15K/well and cultured for one day in 100 μl RPMI1640 culture medium. Fresh PBMCs (0.3×10⁶/well) were added to cancer cells in the presence of different concentrations of antibodies. After incubation for one day, the viable cell population in each well was determined by adding CCK-8 reagent, and reading at OD450 nm. FIG. 3A & 3B show the anti-HER2 antibodies mediated killing of AU-565 and SKBR3 cells, two Breast cancer cell lines, in the presence of fresh PBMCs. The IC50 shown in the table below.

TABLE 2
IC50 (nM) of lead anti-HER2 clones in killing
cancer cells in the presence of PBMCs

| Clones | IC50 (nM) in AU-565 cells | IC50 (nM) in SKBR3 cells |
|---|---|---|
| 4A8 | 0.19 | 0.14 |
| 11F6 | 0.22 | 0.21 |
| 11D5 | 0.38 | 0.29 |
| Perjeta | 0.24 | 0.29 |

### Effects of Lead Anti-HER2 VHH-Fc Clones on Proliferation of HER2 Expressing Breast Cancer Cells

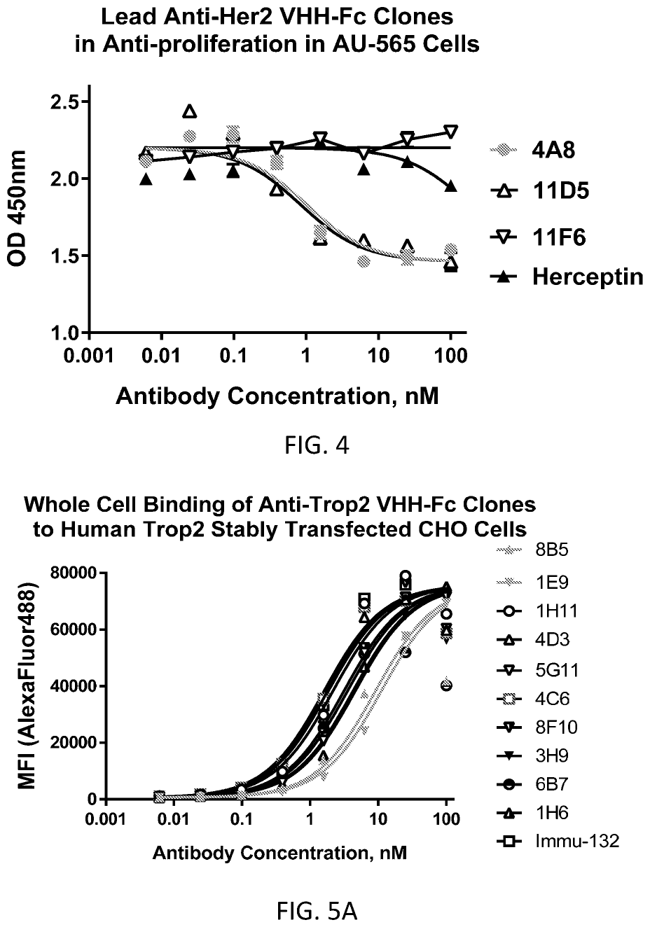

Activation of HER2 can lead to proliferation of certain cancer cells with high expression of HER2. Anti-HER2 antibodies can inhibit the proliferative effect by blocking HER2 activation. To determine the effect of lead anti-HER2 antibodies on proliferation, AU565 cells were cultured with different concentrations of lead anti-HER2 VHH-Fc clones or the reference antibody Herceptin. As shown in FIG. 4, both 4A8 and 11D5 inhibited proliferation of AU-565 cells in a concentration-dependent manner. However, the reference antibody Herceptin only showed a very weak anti-proliferative effect, and 11F6 did not show any anti-proliferative effect in AU-565 cells. The IC50 of anti-proliferative effects of these antibodies shown in the table below.

TABLE 3
Effect of lead anti-HER2 clones in
anti-proliferation in AU-565 cells

| Clones | IC50 (nM) in AU-565 cells |
|---|---|
| 4A8 | 1.02 |
| 11F6 | UD |
| 11D5 | 0.84 |
| Herceptin | UD |

UD: unable to determine.

The CMC profiles of 4A8, 11F6 and 11D5 were evaluated by SEC-HPLC. 11D5 showed very good SEC-HPLC profile with monomer>98%. However, 4A8 and 11F6 showed very poor SEC profile with monomer less than 30% (not shown). Thus, 4A8 and 11F6 were not selected due to poor developability. The sequences of 11D5 are shown in the table below.

TABLE 4
Sequences of lead anti-HER2
VHH clone 11D5 (IMGT definition)

| Clone | | SEQ |
|---|---|---|
| name | FR1 CDR1 FR2 CDR2 FR3 CDR3 FR4 | ID |
| 11D5 | QVQLVESGGGVVQPGRSLRLSCAASGSTFSSLAM | 1 |
| | HWVRQAPGKERELVAAISYSGGYTYYADSVKGRF | |
| | TISRDNSKNTLYLQMNSLRAEDTAVYYCAASSIF | |
| | IGAYDYWGQGTLVTVSS | |

### Example 3: Anti-Human TROP2 VHH Antibodies-Immunization, Library Construction, Panning and Hits Identification

### Immunization and Library Construction

Recombinant human and cynomolgus TROP2 extracellular domain (ECD) were purchased from Sinobiological USA. Immunization of TROP2 antigens was performed using a 2 year's old naïve female Alpaca from Capralogics Inc. The titer of serum antibodies was measured by ELISA assays. After 4 rounds of immunization, a high titer (1:50,000) of anti-human-TROP2 was observed. 80 ml of whole blood was collected from the Alpaca, and PBMCs were isolated. Thereafter RNA was isolated from the PBMCs.

The VHH (variable domain of the heavy chain of heavy-chain antibodies) genes were amplified with PCR using 10 pairs of specifically designed primers covering all Alpaca germline sequences. The PCR products were purified (by DNA agarose gel & gel extraction kit), and cloned into a pADL-23c phagemid library, which is used to transform TG1 cells by electroporation. Transformed TG1 library diversity was 2.7×10⁹. Transformed TG1 cells were cultured in 2YT medium and phages displaying VHH was produced by adding helper phage and culturing overnight. Phages in the supernatant were harvested by precipitation with 4% PGE/0.5M NaCl and high-speed centrifugation. A standard protocol was used to purify the phage library. Final titer of phage library measured at OD260, with 1×10¹³/ml.

### Panning and Hits Identification

Panning of binders to human TROP2 (h-TROP2) antigen was performed using streptavidin-coupled Dynabeads coated with biotinylated h-TROP2 ECD protein. After 2-3 rounds of panning, binders of h-TROP2 were eluted and used to infect SS320 cells. Colonies of SS320 cells were picked and cultured in 2YT medium. 0.5 mM IPTG was added to induce secretion of VHH antibodies. Supernatants with VHH antibodies were screened by flow cytometry whole cell binding assays using h-TROP2 stably transfected CHO cells. In total, 74 whole cell binders were obtained. After sequencing, 25 unique sequences were obtained. The CDR and framework sequences were determined using IMGT (the international ImMunoGeneTics information system).

### Example 4: Characterization of TROP2 VHH Antibodies

### Construction of Bivalent VHH-Fc Clones

25 unique clones from hits identification were used to make bivalent antibodies with human IgG1 Fc region (VHH-Fc) including the hinge region and constant domains (CH2 and CH3). Constructed bivalent VHH-Fc antibodies were transiently expressed in ExpiCHO cells, and proteins in supernatants were harvested and purified by Protein A resin.

### Characterization of TROP2 VHH Antibodies

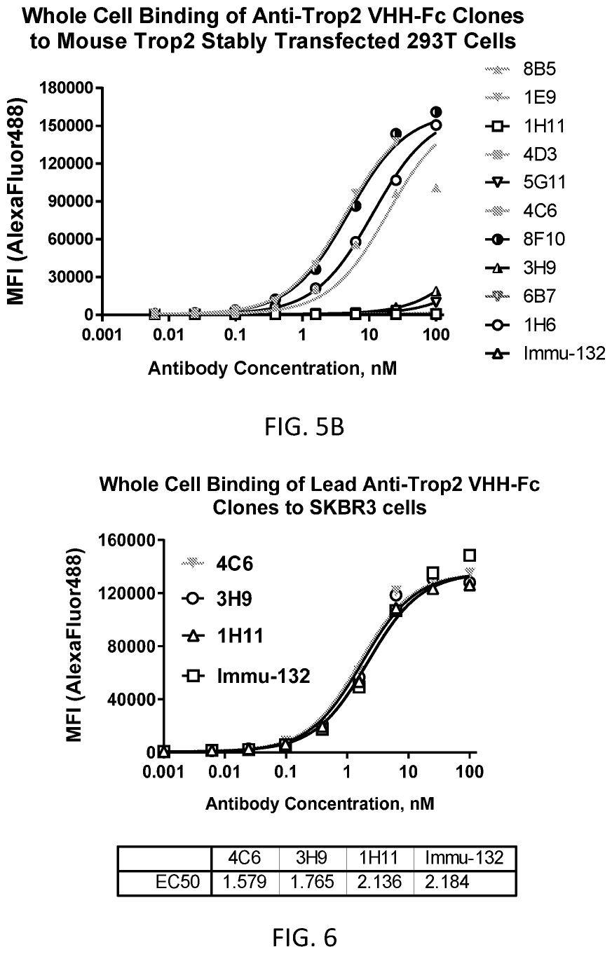

Binding of bivalent anti-TROP2 VHH-Fc clones to human TROP2 in human TROP2 stably transfected CHO cells (CHO-h-TROP2 stable cells) were determined by flow cytometry whole cell binding (WCB) assays. Briefly, antibodies were incubated at different concentrations with CHO-h-TROP2 stable cells (0.2×10⁶/ml) in 96-well plates (100 μl/well) in FACS buffer for 30 min. After washing, Alexa Fluor 488 conjugated anti-human IgG Fc secondary antibody was added, and incubated for 30 min. After washing, MFI was measured using CytoFlex by gating live cell population with FITC channel. EC50 of binding was calculated using GraphPad Prism7.0. A representative figure is shown in FIG. 5A. Similarly, whole cell binding of anti-TROP2 VHH-Fc clones to mouse TROP2 (m-TROP2) was determined using mouse TROP2 stably transfected 293T cells (293T-m-TROP2 cells), and the results are shown in FIG. 5B. EC50 of whole cell binding is shown in the table below.

TABLE 5
Whole cell binding of anti-TROP2 VHH-Fc
clones to human or mouse TROP2

| | EC50 (nM) of WCB to | EC50 (nM) of WCB to |
|---|---|---|
| | h-TROP2 stably | m-TROP2 stably |
| Clone name | transfected CHO cells | transfected 293T cells |
| 4C6 | 1.37 | UD |
| 3H9 | 1.46 | 760.5 |
| 1H11 | 1.84 | UD |
| 5G11 | 3.00 | 1577 |
| 4D3 | 3.53 | UD |
| 8F10 | 3.76 | 4.89 |
| 1H6 | 4.42 | 11.37 |
| 6B7 | 5.33 | UD |
| 8B5 | 7.41 | 18.85 |
| 4D2 | 7.57 | ND |
| 8A2 | 8.98 | ND |
| 1E9 | 9.61 | 3.67 |
| 2E1 | 10.53 | 79.3 |
| 1D2 | 14.44 | ND |
| 8D11 | 19.89 | ND |
| 1D7 | 48.98 | ND |
| 5D12 | 56.99 | ND |
| 4A2 | 59.10 | ND |
| 2E11 | 62.17 | ND |
| 2B11 | 98.50 | ND |
| 4A7 | 108.70 | ND |
| 5C11 | 971.90 | ND |
| 11C6 | 1157.00 | ND |
| Immu-132 | 2.20 | UD |

UD: unable to determine
ND: not determined

The binding affinities of the top three clones (4C6, 3H9 & 1H11) to human cancer cells with high levels of endogenously expressed TROP2 were also determined. As shown in FIG. 6, these lead VHH-Fc clones all bind to TROP2 endogenously expressed in breast cancer cell line SKBR3 with high binding affinities similar to or slightly higher than that of the reference antibody Immu-132, an anti-TROP2 antibody in Trodelvy (from Gilead Sciences). The EC50 of whole cell binding in SKBR3 cells is shown in FIG. 6.

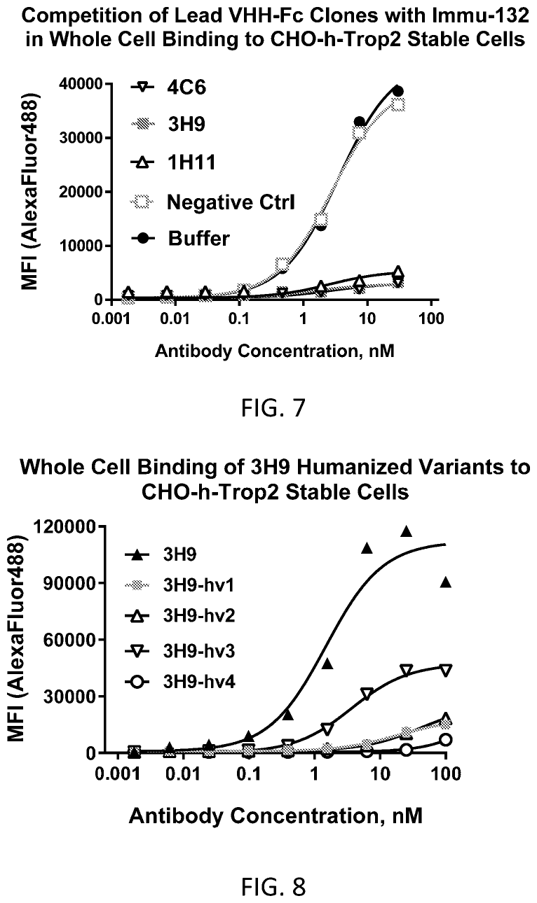

The binding region on human TROP2 was also examined by competition binding assay. Briefly, clone 4C6, 3H9 & 1H11 in 60 nM were incubated with CHO-h-TROP2 stable cells in FACS buffer for 30 min at 4° C. in a 96-well plate. After washing twice, cells were incubated with Immu-132 at different concentrations for 30 min at 4° C. The cells were washed twice again and resuspended in a buffer containing anti-human F(ab)2 Alexa Fluor 488 (1:600 dilution) and incubated for 30 min at 4° C. Cells were then washed once and resuspended in FACS buffer, and MFI was measured using CytoFlex by gating live cell population with a FITC channel. As shown in FIG. 7, all three lead VHH-Fc clones compete with Immu-132 in binding to cell surface human TROP2, indicating they share a similar binding region with Immu-132. Sequences of 3 lead VHH clones are shown in the table below.

TABLE 6
Sequences of anti-TROP2
VHH clones (IMGT definition)

| Clone | | SEQ |
|---|---|---|
| name | FR1 CDR1 FR2 CDR2 FR3 CDR3 FR4 | ID |
| Alpaca | LVQLVESGGGLVQAGGSQRLSCAVSGRAFAYYAM | 2 |
| 3H9 | GWFRQAPGKEREFVAAIAGSGDSTYYADSVKGRF | |
| | TISRDYAKNTNYLQMNSLKPEDTAVYYCAAAGGT | |
| | FYSGTAVATRSSQYDYWGQGTQVTVSS | |
| Alpaca | QVQVVESGGGLVQAGGSQRLSCAVSGHPFAMYAM | 3 |
| 4C6 | GWFRQAPGKEREFVAAIAGNGGSTSYADSVKGRF | |
| | TISRDYAKNTNYLQMNSLEPEDTAVYYCAAAGGT | |
| | FYSGTTVATRSSHYDYWGQGTQVTVSS | |
| Alpaca | LVQLVESGGGLVQAGDSLRLSCTASGGTFSRYII | 4 |
| 1H11 | GWFRQAPGKEREFVTTISGSGGSTYYADSVKGRF | |
| | TISRDNAKNTVFLQMNSLKPEDTAVYYCASTFYS | |
| | TTYVAQRDARDYAYWGQGTQVTVSS | |

### Example 5: Humanization of Lead Anti-TROP2 VHH Clones

Humanization was performed by analyzing germline sequences of top 3 clones using Igblast. The closest human germline (IGHV-2-23*04) sequence was used to modify the Alpaca frameworks of these 3 clones. Four humanized variants were made for 3H9 with minor differences in Framework 2, and fused with human IgG1 Fc. Whole cell binding assay to CHO-h-TROP2 stable cells was performed to compare the binding activity of humanized VHH-Fc variants to the parental Alpaca clone 3H9-Fc. As shown in FIG. 8, all humanized VHH-Fc variants have a lower binding activity compared to parental control, with hv3 showing the best binding among the humanized VHH-Fc variants.

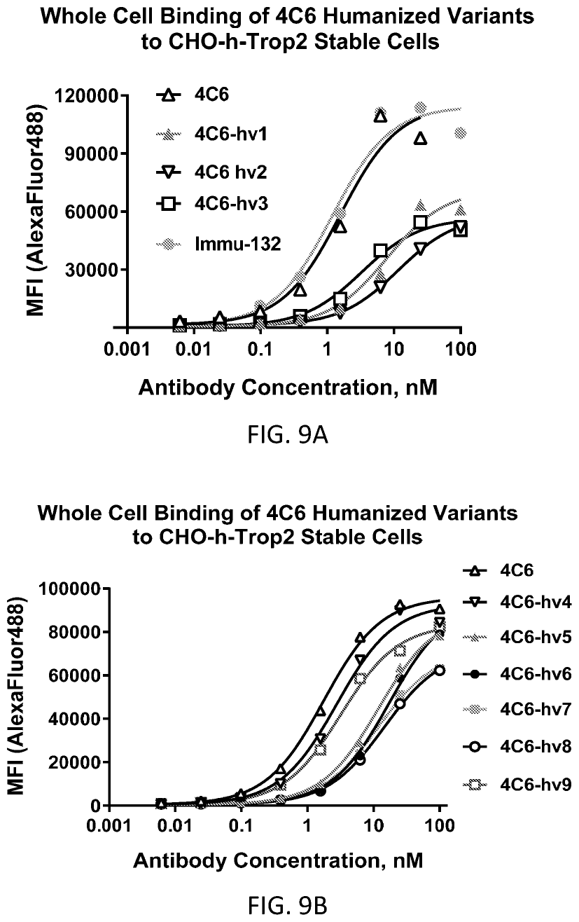

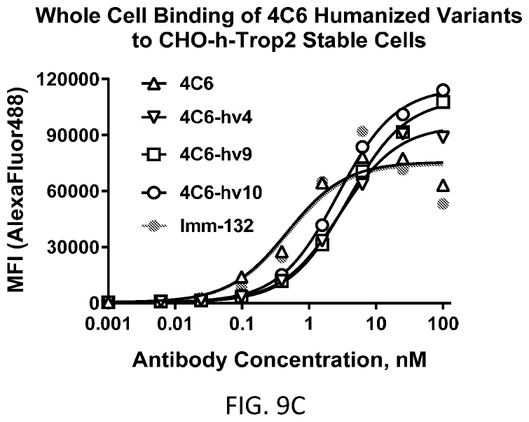

The same strategy was carried out for the humanization of Alpaca clone 4C6. Ten humanized VHH-Fc variants were made for 4C6 with minor difference in Framework 2 and Framework 3. Whole cell binding to CHO-h-TROP2 stable cells was performed to compare the binding affinities of these humanized VHH-Fc variants to the parental Alpaca clone 4C6. Results are shown in FIG. 9A-9C. The EC50 (nM) of whole cell binding is shown in the table below. The sequences of 4C6-hv4, hv9 & hv10 are shown in Table 8.

TABLE 7
EC50 of whole cell binding of humanized variants
of 4C6 to CHO-h-TROP2 stable cells

| | EC50 (nM) of WCB to CHO- |
|---|---|
| Clones | h-TROP2 stable cells |
| 4C6 Alpaca | 0.97 |
| 4C6-hv1 | 21.71 |
| 4C6-hv2 | 56.62 |
| 4C6-hv3 | 26.53 |
| 4C6-hv4 | 2.60 |
| 4C6-hv5 | 12.43 |
| 4C6-hv6 | 15.67 |
| 4C6-hv7 | 22.58 |
| 4C6-hv8 | 27.33 |
| 4C6-hv9 | 2.32 |
| 4C6-hv10 | 1.46 |
| Immu-132 | 0.84 |

TABLE 8
Sequences of selected 4C6
humanized variants (IMGT definition)

| | | S [symbol] |
|---|---|---|
| Clone | FR1 CDR1 FR2 CDR2 FR3 CDR3 FR4 | I [symbol] |
| Alpaca | QVQVVESGGGLVQAGGSQRLSCAVSGHPFAMYAM | [symbol] |
| 4C6 | GWFRQAPGKEREFVAAIAGNGGSTSYADSVKGRF | |
| | TISRDYAKNTNYLQMNSLEPEDTAVYYCAAAGGT | |
| | FYSGTTVATRSSHYDYWGQGTQVTVSS | |
| 4C6- | EVQLVESGGGLVQPGGSLRLSCAASGHPFAMYAM | [symbol] |
| hv4 | GWFRQAPGKEREFVSAIAGNGGSTSYADSVKGRF | |
| | TISRDNSKNTLYLQMNSLRAEDTAVYYCAAAGGT | |
| | FYSGTTVATRSSHYDYWGQGTQVTVSS | |
| 4C6- | EVQLVESGGGLVQPGGSLRLSCAASGHPFAMYAM | [symbol] |
| hv9 | GWFRQAPGKEREFVSAIAGNGGSTYYADSVKGRF | |
| | TISRDNSKNTLYLQMNSLRAEDTAVYYCAAAGGT | |
| | FYSGTTVATRSSHYDYWGQGTQVTVSS | |
| 4C6- | EVQLVESGGGLVQPGGSLRLSCAASGHPFAMYAM | [symbol] |
| hv10 | GWFRQAPGKEREFVAAIAGNGGSTSYADSVKGRF | |
| | TISRDNSKNTLYLQMNSLRAEDTAVYYCAAAGGT | |
| | FYSGTTVATRSSHYDYWGQGTQVTVSS | |

[symbol]

indicates data missing or illegible when filed

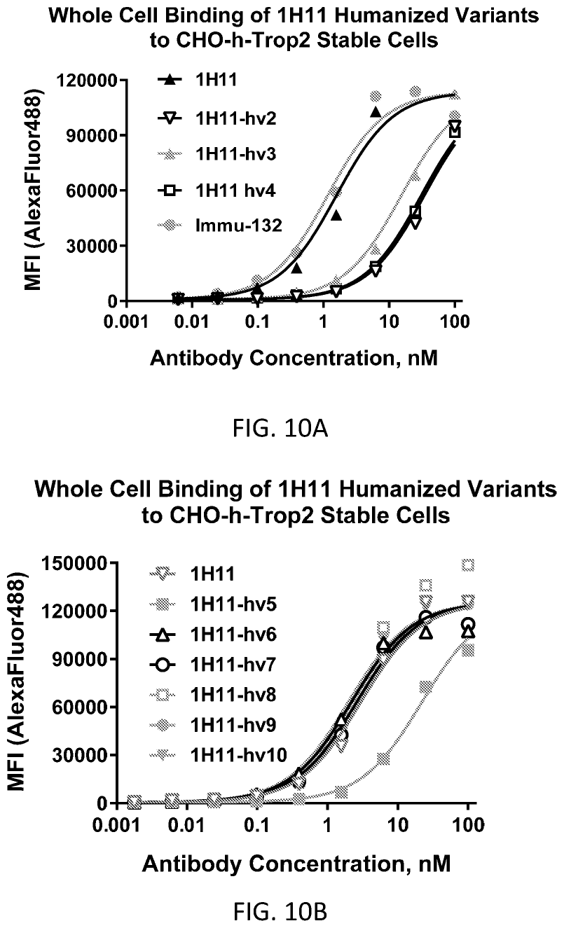

Humanization of Alpaca clone 1H11 was carried out using the same strategy. Ten humanized VHH-Fc variants were made with minor difference in Framework 2. The binding activity of humanized variants to human TROP2 was examined using whole cell binding assays. As shown in FIGS. 10A & 10B, significantly weaker binding was observed for variants hv1, 2, 3, 4, & 5 compared to parental Alpaca clone 1H11. As shown in FIG. 10B and the table below, variants hv6, 7, 8, 9, & 10 retained the binding affinity of Alpaca clone 1H11.

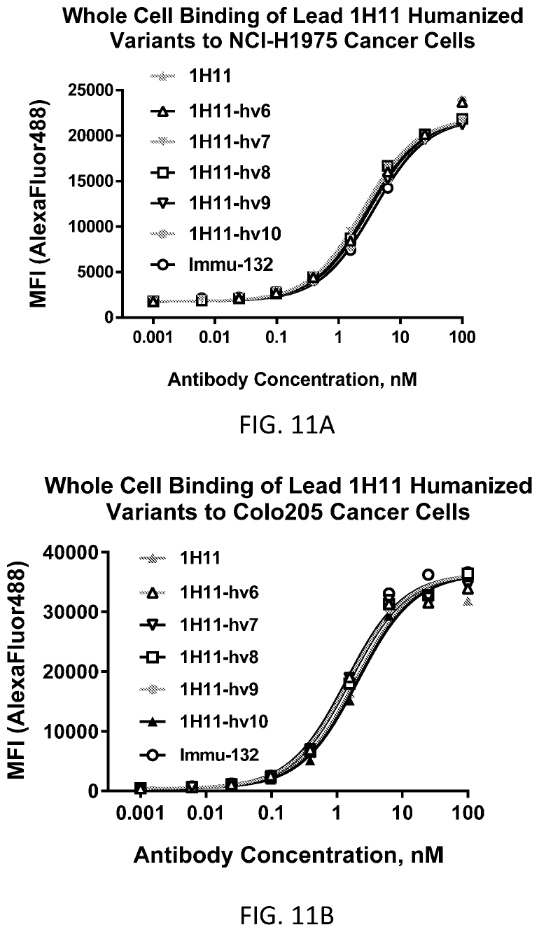

Whole cell binding to human TROP2 endogenously expressed in tumor cells was also examined for selected humanized variants with high binding affinities to TROP2. As shown in FIGS. 11A & B, these humanized variants all retained binding affinities to endogenously expressed human TROP2 in tumor cells NCI-H1975 (human lung cancer cells) & Colo205 (human gastric cancer cells), compared to the parental Alpaca clone 1H11, with similar binding affinities to that of the reference antibody Immu-132. The EC50 values are shown in the table below.

TABLE 9
EC50 of whole cell binding of humanized variants of
1H11 to CHO-h-TROP2 stable cells & their CMC profiles

| | EC50 (nM) of | | | | |
|---|---|---|---|---|---|
| | WCB to | EC50 (nM) of | EC50 (nM) of | | SEC |
| | CHO-h-TROP2 | WCB to NCI- | WCB to | | HPLC |
| Clones | stable cells | H1975 cells | Colo205 cells | Tm | Monomer |
| Alpaca 1H11 | 1.57 | 2.59 | 1.53 | 67.8 | 99.3 |
| 1H11-hv2 | 34.23 | ND | ND | ND | ND |
| 1H11-hv3 | 15.36 | ND | ND | ND | ND |
| 1H11-hv4 | 30.77 | ND | ND | ND | ND |
| 1H11-hv5 | 22.02 | ND | ND | ND | ND |
| 1H11-hv6 | 2.18 | 2.70 | 1.22 | 59.0 | 98.3 |
| 1H11-hv7 | 2.59 | 2.42 | 1.19 | 63.4 | 89.7 |
| 1H11-hv8 | 1.97 | 2.54 | 1.29 | 68.1 | 97.4 |
| 1H11-hv9 | 2.73 | 3.04 | 1.29 | 67.2 | 97.5 |
| 1H11-hv10 | 2.29 | 3.12 | 1.71 | 68.8 | 98.3 |
| Immu-132 | 1.16 | 3.61 | 1.15 | 69.1 | 97.5 |

ND: not determined

Thermal shift and SEC-HPLC were performed for these potent humanized variants, as shown in the table above, 1H11-hv6 and 1H11-hv7 have lower Tm compared to other humanized variants. The SEC-HPLC profiles are similar for most of these potent humanized variants (over 9500 monomer ratio except 1H11-hv7), indicating good stability & developability for most potent humanized variants. Sequences of selected humanized variants of 1H11 are shown in the table below.

TABLE 10
Sequences of selected humanized
variants of 1H11 (IMGT definition)

| | | SEQ |
|---|---|---|
| Clone | FR1 CDR1 FR2 CDR2 FR3 CDR3 FR4 | ID |
| Alpaca | LVQLVESGGGLVQAGDSLRLSCTASGGTFSRYIIGW | 4 |
| 1H11 | FRQAPGKEREFVTTISGSGGSTYYADSVKGRFTISR | |
| | DNAKNTVFLQMNSLKPEDTAVYYCASTFYSTTYVAQ | |
| | RDARDYAYWGQGTQVTVSS | |
| 1H11- | EVQLVESGGGLVQPGGSLRLSCAASGGTFSRYIIGW | 8 |
| hv6 | FRQAPGKEREFVTTISGSGGSTYYADSVKGRFTISR | |
| | DNSKNTLYLQMNSLRAEDTAVYYCASTFYSTTYVAQ | |
| | RDARDYAYWGQGTQVTVSS | |
| 1H11- | EVQLVESGGGLVQPGGSLRLSCAASGGTFSRYIIGW | 9 |
| hv7 | FRQAPGKEREFVSTISGSGGSTYYADSVKGRFTISR | |
| | DNSKNTLYLQMNSLRAEDTAVYYCASTFYSTTYVAQ | |
| | RDARDYAYWGQGTQVTVSS | |
| 1H11- | EVQLVESGGGLVQPGGSLRLSCAASGGTFSRYIMGW | 10 |
| hv8 | FRQAPGKEREFVSTISGSGGSTYYADSVKGRFTISR | |
| | DNSKNTLYLQMNSLRAEDTAVYYCASTFYSTTYVAQ | |
| | RDARDYAYWGQGTQVTVSS | |
| 1H11- | EVQLVESGGGLVQPGGSLRLSCAASGGTFSRYIMSW | 11 |
| hv9 | FRQAPGKEREFVSTISGSGGSTYYADSVKGRFTISR | |
| | DNSKNTLYLQMNSLRAEDTAVYYCASTFYSTTYVAQ | |
| | RDARDYAYWGQGTQVTVSS | |
| 1H11- | EVQLVESGGGLVQPGGSLRLSCAASGGTFSRYIMSW | 12 |
| hv10 | FRQAPGKEREFVSAISGSGGSTYYADSVKGRFTISR | |
| | DNSKNTLYLQMNSLRAEDTAVYYCASTFYSTTYVAQ | |
| | RDARDYAYWGQGTQVTVSS | |

### Example 6: Construction of Anti-HER2/TROP2 Bispecific Antibodies

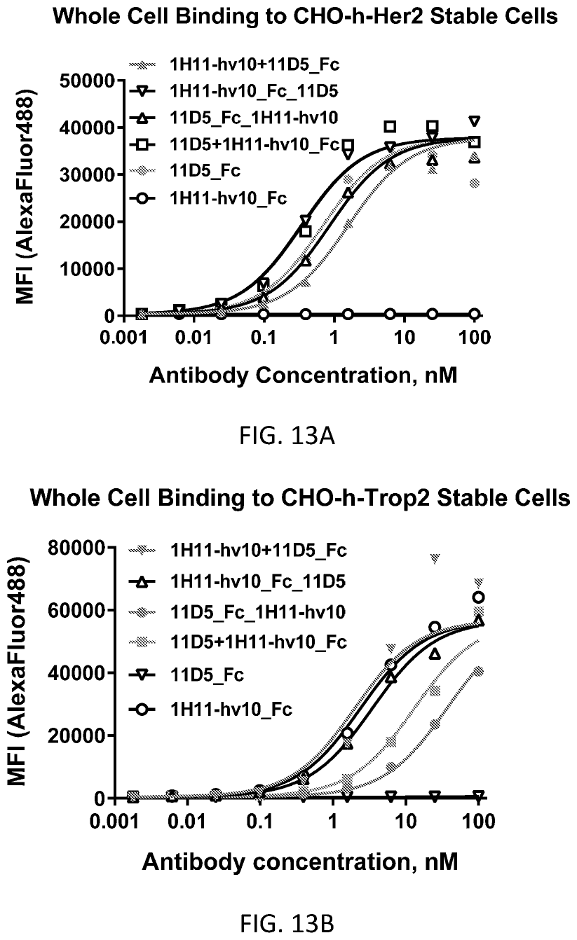

Anti-HER2 VHHT clone 11D5 was selected to build the bispecific antibody based on its binding affinity, potency and CMC profile. Anti-TROP2 VHHT clone 1H11-hv10 was selected initially to build the bispecific antibodies to test in vitro functions. To reduce the number of disulfide-bonds in the hinge region for VHH-Fc or BsAb constructs, and to obtain desired number of Payload/Toxin conjugation, the first Cysteine in the hinge region was changed to Serine (C220S), leading to mutated constructs only containing 2 Cysteines in each hinge region (one heavy chain) to form 2 disulfide-bonds for these VHH-Fc/BsAbs. To examine the impact of different formats of BsAbs in binding to both tumor target HER2 and TROP2, four different formats of BsAbs were made as shown in FIGS. 12C-12F. Whole cell binding of BsAbs to human HER2 and TROP2 was determined and binding curves are shown in FIGS. 13A & 13B. EC50 (nM) values are shown in the table below. The results indicate that both 1H11-hv10+11D5_Fc (tandem) and 1H11-hv10_Fc_11D5 (N-, C-terminal) formats can retain binding affinities to both HER2 and TROP2, with the 1H11-hv10_Fc_11D5 format having a higher binding affinity to HER2 than that of the 1H11-hv10+11D5_Fc format.

TABLE 11
Summary of WCB of BsAbs to stably transfected cells

| | EC50 (nM) in | EC50 (nM) in |
|---|---|---|
| | WCB to | WCB to |
| | CHO-h-HER2 | CHO-h-TROP2 |
| Clones | stable cells | stable cells |
| 11D5_Fc | 0.684 | / |
| 1H11-hv10_Fc | / | 2.38 |
| 1H11-hv10 + 11D5_Fc | 1.57 | 1.98 |
| 1H11-hv10_Fc_11D5 | 0.335 | 3.46 |
| 11D5 + 1H11-hv10_Fc | 0.34 | 13.04 |
| 11D5_Fc_1H11-hv10 | 0.85 | 36.62 |

Sequences of these mono- & bispecific antibodies are shown in Table 12 & 13, all with C220S mutation in the hinge region.

TABLE 12
Sequences of monospecific VHH-Fc antibodies

| | | Full |
|---|---|---|
| | | Length |
| Clones of Monospecific Antibody | | SEQ ID |
| 11D5_Fc | | |
| 11D5 | Hinge + CH2 + CH3 (IgG1) | |
| QVQLVESGGGVVQPGRSLRLSCAA | EPKSSDKTHTCPPCPAPELLGGPS | 13 |
| SGSTFSSLAMHWVRQAPGKERELV | VFLFPPKPKDTLMISRTPEVTCVV | |
| AAISYSGGYTYYADSVKGRFTISR | VDVSHEDPEVKFNWYVDGVEVHNA | |
| DNSKNTLYLQMNSLRAEDTAVYYC | KTKPREEQYNSTYRVVSVLTVLHQ | |
| AASSIFIGAYDYWGQGTLVTVSS | DWLNGKEYKCKVSNKALPAPIEKT | |
| (SEQ ID NO: 1) | ISKAKGQPREPQVYTLPPSREEMT | |
| | KNQVSLTCLVKGFYPSDIAVEWES | |
| | NGQPENNYKTTPPVLDSDGSFFLY | |
| | SKLTVDKSRWQQGNVFSCSVMHEA | |
| | LHNHYTQKSLSLSPGK | |
| | (SEQ ID NO: 36) | |
| 1H11-hv10_Fc | | |
| 1H11-hv10 | Hinge + CH2 + CH3 (IgG1) | |
| EVQLVESGGGLVQPGGSLRLSCAA | EPKSSDKTHTCPPCPAPELLGGPS | 14 |
| SGGTFSRYIMSWFRQAPGKEREFV | VFLFPPKPKDTLMISRTPEVTCVV | |
| SAISGSGGSTYYADSVKGRFTISR | VDVSHEDPEVKFNWYVDGVEVHNA | |
| DNSKNTLYLQMNSLRAEDTAVYYC | KTKPREEQYNSTYRVVSVLTVLHQ | |
| ASTFYSTTYVAQRDARDYAYWGQG | DWLNGKEYKCKVSNKALPAPIEKT | |
| TQVTVSS (SEQ ID NO: 12) | ISKAKGQPREPQVYTLPPSREEMT | |
| | KNQVSLTCLVKGFYPSDIAVEWES | |
| | NGQPENNYKTTPPVLDSDGSFFLY | |
| | SKLTVDKSRWQQGNVFSCSVMHEA | |
| | LHNHYTQKSLSLSPGK | |
| | (SEQ ID NO: 36) | |

TABLE 13
Sequences of bispecific antibodies in different formats

| | | | | Full |
|---|---|---|---|---|
| | | | | Length |
| Clones of Bispecific Antibody | | | | SEQ ID |
| 1H11-hv10 + 11D5_Fc | | | | |
| | | | Hinge + CH2 + | |
| 1H11-hv10 | Linker1 | 11D5 | CH3 (IgG1) | |
| EVQLVESGGGLV | GSGGSGGSGGSG | QVQLVESGGGV | EPKSSDKTHTCPPCP | 15 |
| QPGGSLRLSCAA | (SEQ ID NO: 34) | VQPGRSLRLSC | APELLGGPSVFLFPP | |
| SGGTFSRYIMSW | | AASGSTFSSLA | KPKDTLMISRTPEVT | |
| FRQAPGKEREFV | | MHWVRQAPGKE | CVVVDVSHEDPEVKF | |
| SAISGSGGSTYY | | RELVAAISYSG | NWYVDGVEVHNAKTK | |
| ADSVKGRFTISR | | GYTYYADSVKG | PREEQYNSTYRVVSV | |
| DNSKNTLYLQMN | | RFTISRDNSKN | LTVLHQDWLNGKEYK | |
| SLRAEDTAVYYC | | TLYLQMNSLRA | CKVSNKALPAPIEKT | |
| ASTFYSTTYVAQ | | EDTAVYYCAAS | ISKAKGQPREPQVYT | |
| RDARDYAYWGQG | | SIFIGAYDYWG | LPPSREEMTKNQVSL | |
| TQVTVSS | | QGTLVTVSS | TCLVKGFYPSDIAVE | |
| (SEQ ID | | (SEQ ID | WESNGQPENNYKTTP | |
| NO: 12) | | NO: 1) | PVLDSDGSFFLYSKL | |
| | | | TVDKSRWQQGNVFSC | |
| | | | SVMHEALHNHYTQKS | |
| | | | LSLSPGK | |
| | | | (SEQ ID NO: 36) | |
| 1H11-hv10_Fc_11D5 | | | | |
| | Hinge + CH2 + | | | |
| 1H11-hv10 | CH3 (IgG1) | Linker2 | 11D5 | |
| EVQLVESGGGLV | EPKSSDKTHTCPPCPAPELL | GSGGSGGSGGS | QVQLVESGGGVVQPG | 16 |
| QPGGSLRLSCAA | GGPSVFLFPPKPKDTLMISR | GGSG | RSLRLSCAASGSTFS | |
| SGGTFSRYIMSW | TPEVTCVVVDVSHEDPEVKF | (SEQ ID | SLAMHWVRQAPGKER | |
| FRQAPGKEREFV | NWYVDGVEVHNAKTKPREEQ | NO: 35) | ELVAAISYSGGYTYY | |
| SAISGSGGSTYY | YNSTYRVVSVLTVLHQDWLN | | ADSVKGRFTISRDNS | |
| ADSVKGRFTISR | GKEYKCKVSNKALPAPIEKT | | KNTLYLQMNSLRAED | |
| DNSKNTLYLQMN | ISKAKGQPREPQVYTLPPSR | | TAVYYCAASSIFIGA | |
| SLRAEDTAVYYC | EEMTKNQVSLTCLVKGFYPS | | YDYWGQGTLVTVSS | |
| ASTFYSTTYVAQ | DIAVEWESNGQPENNYKTTP | | (SEQ ID NO: 1) | |
| RDARDYAYWGQG | PVLDSDGSFFLYSKLTVDKS | | | |
| TQVTVSS | RWQQGNVFSCSVMHEALHNH | | | |
| (SEQ ID | YTQKSLSLSPGK | | | |
| NO: 12) | (SEQ ID NO: 36) | | | |
| 11D5 + 1H11-hv10_Fc | | | | |
| | | | Hinge + CH2 + | |
| 11D5 | Linker1 | 1H11-hv10 | CH3 (IgG1) | |
| QVQLVESGGGVV | GSGGSGGSGGSG | EVQLVESGGGL | EPKSSDKTHTCPPCP | 17 |
| QPGRSLRLSCAA | (SEQ ID NO: 34) | VQPGGSLRLSC | APELLGGPSVFLFPP | |
| SGSTFSSLAMHW | | AASGGTFSRYI | KPKDTLMISRTPEVT | |
| VRQAPGKERELV | | MSWFRQAPGKE | CVVVDVSHEDPEVKF | |
| AAISYSGGYTYY | | REFVSAISGSG | NWYVDGVEVHNAKTK | |
| ADSVKGRFTISR | | GSTYYADSVKG | PREEQYNSTYRVVSV | |
| DNSKNTLYLQMN | | RFTISRDNSKN | LTVLHQDWLNGKEYK | |
| SLRAEDTAVYYC | | TLYLQMNSLRA | CKVSNKALPAPIEKT | |
| AASSIFIGAYDY | | EDTAVYYCAST | ISKAKGQPREPQVYT | |
| WGQGTLVTVSS | | FYSTTYVAQRD | LPPSREEMTKNQVSL | |
| (SEQ ID | | ARDYAYWGQGT | TCLVKGFYPSDIAVE | |
| NO: 1) | | QVTVSS | WESNGQPENNYKTTP | |
| | | (SEQ ID | PVLDSDGSFFLYSKL | |
| | | NO: 12) | TVDKSRWQQGNVFSC | |
| | | | SVMHEALHNHYTQKS | |
| | | | LSLSPGK | |
| | | | (SEQ ID NO: 36) | |
| 11D5_Fc_1H11-hv10 | | | | |
| | Hinge + CH2 + | | | |
| 11D5 | CH3 (IgG1) | Linker2 | 1H11-hv10 | |
| QVQLVESGGGVV | EPKSSDKTHTCPPCPAPELL | GSGGSGGSGGS | EVQLVESGGGLVQPG | 18 |
| QPGRSLRLSCAA | GGPSVFLFPPKPKDTLMISR | GGSG | GSLRLSCAASGGTFS | |
| SGSTFSSLAMHW | TPEVTCVVVDVSHEDPEVKF | (SEQ ID | RYIMSWFRQAPGKER | |
| VRQAPGKERELV | NWYVDGVEVHNAKTKPREEQ | NO: 35) | EFVSAISGSGGSTYY | |
| AAISYSGGYTYY | YNSTYRVVSVLTVLHQDWLN | | ADSVKGRFTISRDNS | |
| ADSVKGRFTISR | GKEYKCKVSNKALPAPIEKT | | KNTLYLQMNSLRAED | |
| DNSKNTLYLQMN | ISKAKGQPREPQVYTLPPSR | | TAVYYCASTFYSTTY | |
| SLRAEDTAVYYC | EEMTKNQVSLTCLVKGFYPS | | VAQRDARDYAYWGQG | |
| AASSIFIGAYDY | DIAVEWESNGQPENNYKTTP | | TQVTVSS | |
| WGQGTLVTVSS | PVLDSDGSFFLYSKLTVDKS | | (SEQ ID NO: 12) | |
| (SEQ ID | RWQQGNVFSCSVMHEALHNH | | | |
| NO: 1) | YTQKSLSLSPGK | | | |
| | (SEQ ID NO: 36) | | | |

### Example 7: Characterization of Anti-HER2/TROP2 Bispecific Antibodies

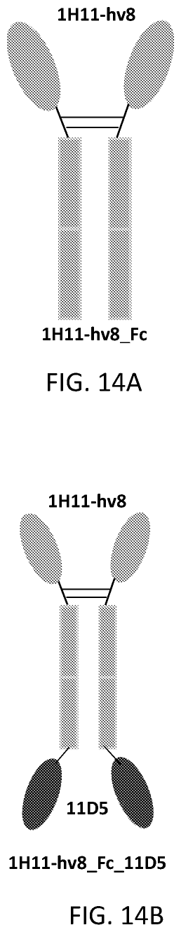

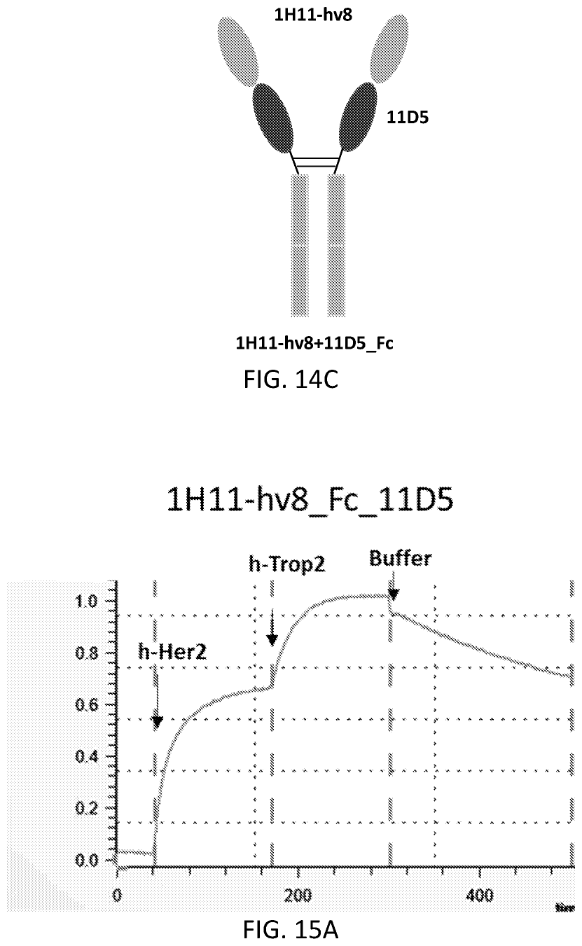

Based on the whole cell binding data of BsAbs in different formats, the N-, C-terminal format (anti-TROP2 in the N-terminal, anti-HER2 in the C-terminal) & the tandem format (anti-TROP2 in the top, anti-HER2 in the middle) were selected to make more BsAbs using humanized variant 1H11-hv8 (bivalent 1H11-hv8_Fc shown in FIG. 14A) of anti-TROP2 VHH and 11D5 of anti-HER2 VHH, fused with human IgG1 Fc, as shown in FIGS. 14B & 14C. Their sequences are shown in the tables below, all with C220S mutation in the hinge region.

TABLE 14
Sequences of monospecific anti-TROP2 antibody

| Clone Name of Monospecific Antibody | | Full |
|---|---|---|
| 1H11-hv8_Fc | | Length |
| 1H11-hv8 | Hinge + CH2 + CH3 (IgG1) | SEQ ID |
| EVQLVESGGGLVQPGGSLRLSCAA | EPKSSDKTHTCPPCPAPELLGGPS | 19 |
| SGGTFSRYIMGWFRQAPGKEREFV | VFLFPPKPKDTLMISRTPEVTCVV | |
| STISGSGGSTYYADSVKGRFTISR | VDVSHEDPEVKFNWYVDGVEVHNA | |
| DNSKNTLYLQMNSLRAEDTAVYYC | KTKPREEQYNSTYRVVSVLTVLHQ | |
| ASTFYSTTYVAQRDARDYAYWGQG | DWLNGKEYKCKVSNKALPAPIEKT | |
| TQVTVSS (SEQ ID NO: 10) | ISKAKGQPREPQVYTLPPSREEMT | |
| | KNQVSLTCLVKGFYPSDIAVEWES | |
| | NGQPENNYKTTPPVLDSDGSFFLY | |
| | SKLTVDKSRWQQGNVFSCSVMHEA | |
| | LHNHYTQKSLSLSPGK | |
| | (SEQ ID NO: 36) | |

TABLE 15
Sequence of bispecific antibodies in different formats

| | | | | Full |
|---|---|---|---|---|
| | | | | Length |
| Clone Names of Bispecific Antibody | | | | SEQ ID |
| 1H11-hv8_Fc_11D5 | | | | |
| | Hinge + CH2 + | | | |
| 1H11-hv8 | CH3 (IgG1) | Linker2 | 11D5 | |
| EVQLVESGGGLV | EPKSSDKTHTCPPCPAPELLGGPS | GSGGSGGSGGS | QVQLVESGGGVVQPGRSL | 20 |
| QPGGSLRLSCAA | VFLFPPKPKDTLMISRTPEVTCVV | GGSG | RLSCAASGSTFSSLAMHW | |
| SGGTFSRYIMGW | VDVSHEDPEVKFNWYVDGVEVHNA | (SEQ ID | VRQAPGKERELVAAISYS | |
| FRQAPGKEREFV | KTKPREEQYNSTYRVVSVLTVLHQ | NO: 35) | GGYTYYADSVKGRFTISR | |
| STISGSGGSTYY | DWLNGKEYKCKVSNKALPAPIEKT | | DNSKNTLYLQMNSLRAED | |
| ADSVKGRFTISR | ISKAKGQPREPQVYTLPPSREEMT | | TAVYYCAASSIFIGAYDY | |
| DNSKNTLYLQMN | KNQVSLTCLVKGFYPSDIAVEWES | | WGQGTLVTVSS | |
| SLRAEDTAVYYC | NGQPENNYKTTPPVLDSDGSFFLY | | (SEQ ID NO: 1) | |
| ASTFYSTTYVAQ | SKLTVDKSRWQQGNVFSCSVMHEA | | | |
| RDARDYAYWGQG | LHNHYTQKSLSLSPGK | | | |
| TQVTVSS | SEQ ID NO: 36) | | | |
| SEQ ID | | | | |
| NO: 10) | | | | |
| 1H11-hv8+11D5_Fc | | | | |
| | | | Hinge + CH2 + | |
| 1H11-hv8 | Linker1 | 11D5 | CH3 (IgG1) | |
| EVQLVESGGGLV | GSGGSGGSGGSG | QVQLVESGGGV | EPKSSDKTHTCPPCPAPE | 21 |
| QPGGSLRLSCAA | (SEQ ID NO: 34) | VQPGRSLRLSC | LLGGPSVFLFPPKPKDTL | |
| SGGTFSRYIMGW | | AASGSTFSSLA | MISRTPEVTCVVVDVSHE | |
| FRQAPGKEREFV | | MHWVRQAPGKE | DPEVKFNWYVDGVEVHNA | |
| STISGSGGSTYY | | RELVAAISYSG | KTKPREEQYNSTYRVVSV | |
| ADSVKGRFTISR | | GYTYYADSVKG | LTVLHQDWLNGKEYKCKV | |
| DNSKNTLYLQMN | | RFTISRDNSKN | SNKALPAPIEKTISKAKG | |
| SLRAEDTAVYYC | | TLYLQMNSLRA | QPREPQVYTLPPSREEMT | |
| ASTFYSTTYVAQ | | EDTAVYYCAAS | KNQVSLTCLVKGFYPSDI | |
| RDARDYAYWGQG | | SIFIGAYDYWG | AVEWESNGQPENNYKTTP | |
| TQVTVSS | | QGTLVTVSS | PVLDSDGSFFLYSKLTVD | |
| SEQ ID | | (SEQ ID | KSRWQQGNVFSCSVMHEA | |
| NO: 10) | | NO: 1) | LHNHYTQKSLSLSPGK | |
| | | | (SEQ ID NO: 36) | |

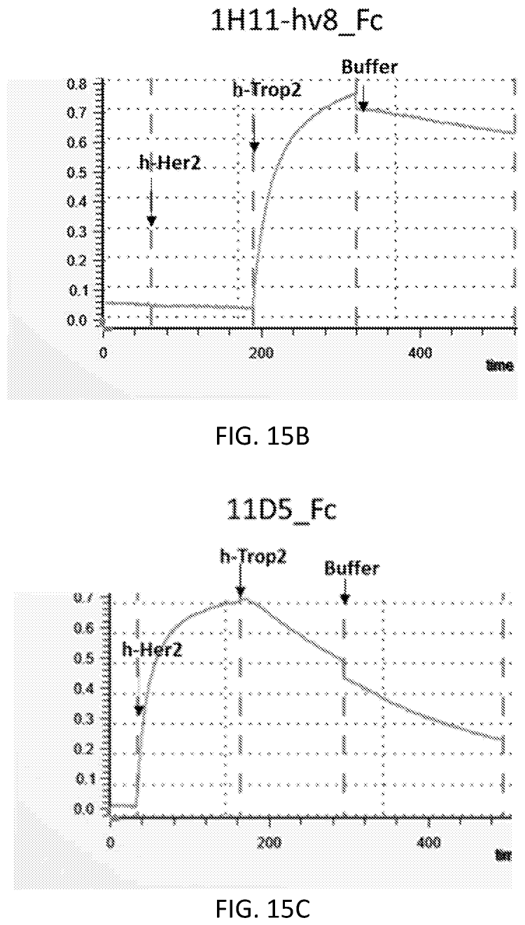

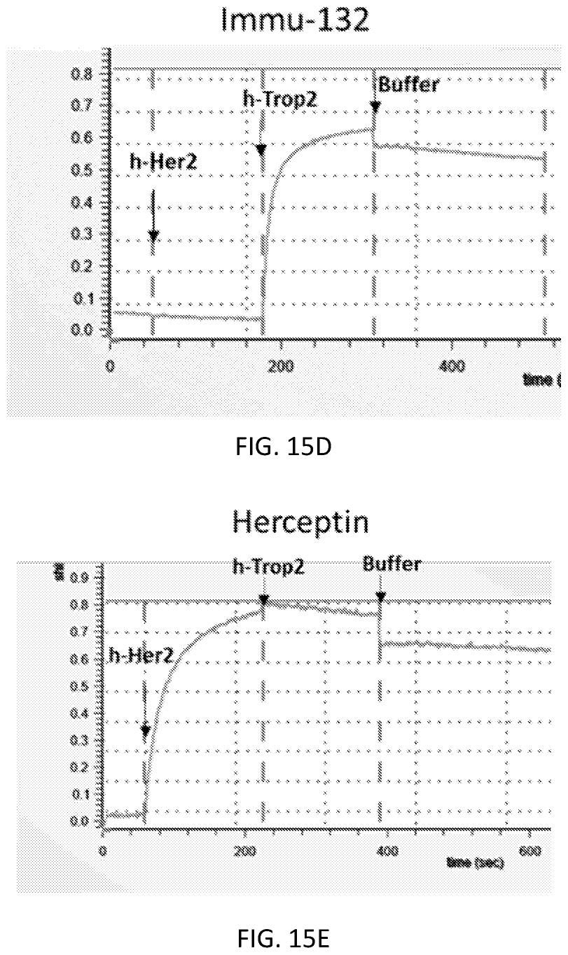

The kinetic binding of the BsAb in N-, C-terminal format (1H11-hv8_Fc_11D5) to two target antigens was determined with monospecific antibodies as controls. As shown in FIGS. 15A-15E, 1H11-hv8_Fc_11D5 binds to both human HER2 & TROP2 with good association, but the monospecific antibodies 1H11-hv8 and Immu-132 only bind to human TROP2, and 11D5 and Herceptin only bind to human HER2.

Antibody-drug conjugates (ADCs) have brought significant benefits to patients as anticancer medications. Among them 4 drugs using vc-MMAE (or mc-vc-PAB-MMAE) have been approved. Vc-MMAE antibody-drug conjugates consist of a monoclonal antibody (mAb) covalently bound to a potent anti-mitotic toxin MMAE (monomethyl auristatin E) through a lysosomal cleavable dipeptide, the valine-citrulline (vc) linker. The bispecific antibody anti-HER2/TROP2 conjugated with MMAE may provide a better treatment option, considering the tumor escape due to expression of a different tumor associated antigen (TAA) that cannot be targeted by monospecific antibody drug-conjugates.

Whole cell binding of two lead BsAbs (1H11-hv8_Fc_11D5 & 1H11-hv8+11D5_Fc) to tumor cells expressing human HER2 or TROP2 at different levels was determined. Potential effects of ADC mediated killing of tumor cells expressing HER2 or TROP2 at different levels was also determined in the presence of a second antibody: the MMAE conjugated anti-human IgG Fc (a Fab) with a cleavable linker (named as 2^{nd} Ab-MMAE. Purchased from MORADEC, AH-202-AE).

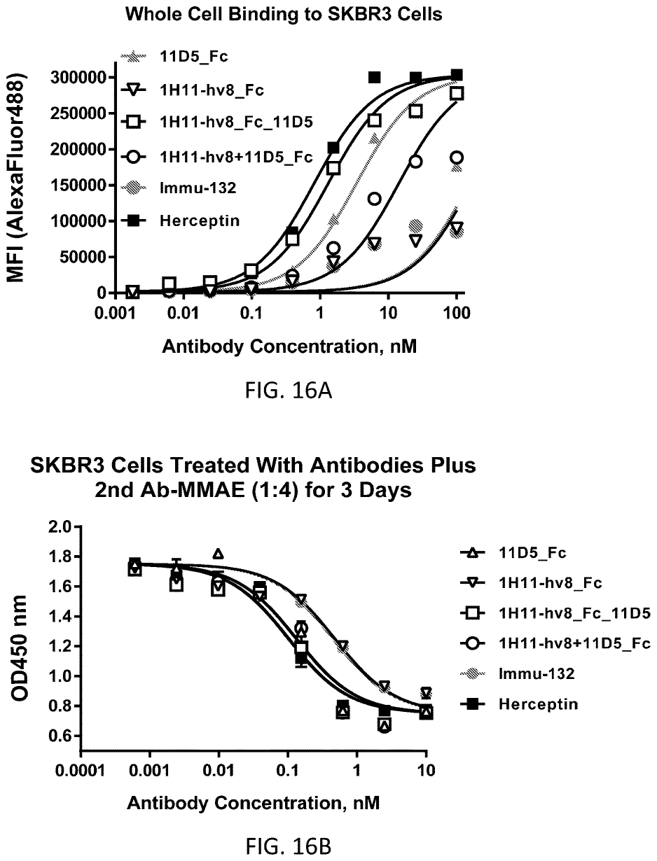

As shown in FIG. 16A, whole cell binding assays showed that SKBR3 cells expressing HER2 at a very high level (MFI around 300,000 as determined by binding of Herceptin at 100 nM), and TROP2 at a high level (MFI around 90,000 as determined by binding of Immu-132 at 100 nM). The N-, C-terminal format BsAb 1H11-hv8_Fc_11D5 has a higher binding affinity to HER2 than that of the tandem format. The binding affinity is similar to that of Herceptin. Killing assay was performed for these antibodies in the presence of 2^{nd} Ab-MMAE, at a ratio of 1:4 or 1:6 (primary antibody vs. 2^{nd} Ab-MMAE), and the amount of viable cells was measured by adding cell counting-8 (CCK-8) reagent and reading at 450 nm 1-4 hours later. Results showed that Herceptin and the N-, C-terminal format BsAb 1H11-hv8_Fc_11D5 have similar potency in killing SKBR3 cells. However, the tandem format BsAb 1H11-hv8+11D5_Fc is slightly weaker in killing SKBR3 cells. The EC50 values are shown in Table 16.

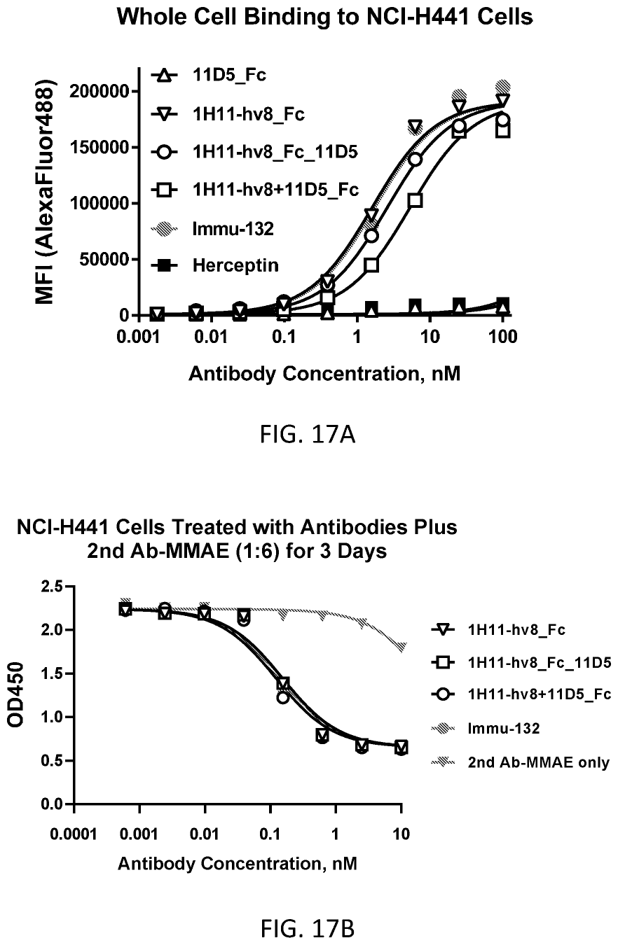

As shown in FIG. 17A, whole cell binding assay showed that NCI-H441 cells, a human lung cancer cell line, express TROP2 at a very high level (MFI around 200,000 as determined by binding of Immu-132 at 100 nM), and express HER2 at a very low level (MFI around 10,000 as determined by binding of Herceptin at 100 nM). In killing assay using the 2^{nd}Ab-MMAE at a ratio of 1:4, BsAbs in both formats showed similar potencies compared to the reference antibody Immu-132. The killing effect is dependent on TROP2 expression levels (FIG. 17B).

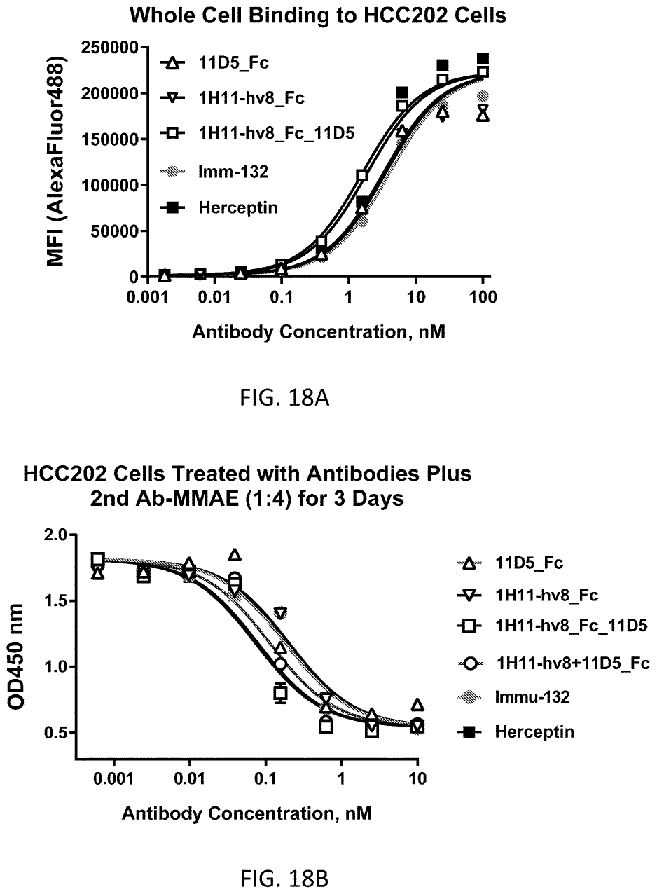

Whole cell binding and killing using the 2^{nd} Ab-MMAE were also performed for HCC202 cells (Breast cancer cells). The cells express both HER2 and TROP2 at high levels as shown in FIG. 18A, with HER2 level (MFI 240,000 as determined by binding of Herceptin at 100 nM) slightly higher than TROP2 level (MFI 200,000 as determined by binding of Immu-132 at 100 nM). The potencies in cell killing mediated by anti-HER2 and TROP2 in the presence of 2^{nd} Ab-MMAE are also similar, as shown in FIG. 18B and the table below.

TABLE 16
EC50 of WCB and IC50 in killing assay in tumor cells expressing
different levels of endogenous HER2 and TROP2.

| | EC50/IC50, nM | | | | | |
|---|---|---|---|---|---|---|
| | | Killing of | | | | Killing of |
| | | SKBR3 cells | | Killing of NCI- | WCB to | HCC202 cells |
| | WCB to | by 2^{nd} Ab- | WCB to NCI- | H441 cells by | HCC202 | by 2nd Ab- |
| Clones | SKBR3 | MMAE | H441 cells | 2^{nd} Ab-MMAE | cells | MMAE |
| 11D5_Fc | 2.59 | 0.134 | UD | ND | 3.25 | 0.18 |
| 1H11-hv8_Fc | 141.2 | 0.48 | 1.60 | 0.15 | 3.5 | 0.20 |
| 1H11- | 9.93 | 0.131 | 5.30 | 0.12 | ND | 0.11 |
| hv8 + 11D5_Fc | | | | | | |
| 1H11- | 0.99 | 0.101 | 2.58 | 0.15 | 1.58 | 0.08 |
| hv8_Fc_11D5 | | | | | | |
| Herceptin | 0.65 | 0.104 | UD | ND | 1.86 | 0.07 |
| Immu-132 | 128.4 | 0.452 | 1.74 | 0.13 | 4.02 | 0.19 |

UD: unable to determine
ND: not determined

### Example 8: Vc-MMAE Conjugation for Lead Anti-HER2 & Anti-TROP2 Monospecific Antibodies and Anti-HER2/TROP2 Bispecific Antibody

The N-, C-terminal format BsAb 1H11-hv8_Fc_11D5 was selected as the lead BsAb to make vc-MMAE (or mv-vc-PAB-MMAE) conjugation. Conjugation was also performed for monospecific bivalent parental clones 11D5_Fc and 1H11-hv8_Fc. As aforementioned, all of these VHH-Fc constructs contain mutation of C220S in the hinge region to reduce Cysteine numbers from 3 to 2, leading to only 2 disulfide-bonds formed in these antibodies, to achieve the goal of Drug-Antibody-Ration (DAR) of 4 for the vc-MMAE conjugation. Conjugation was also performed for reference antibodies Herceptin and Immu-132, with a goal to make DAR around 4 to have a fair comparison in in vitro potency assays.

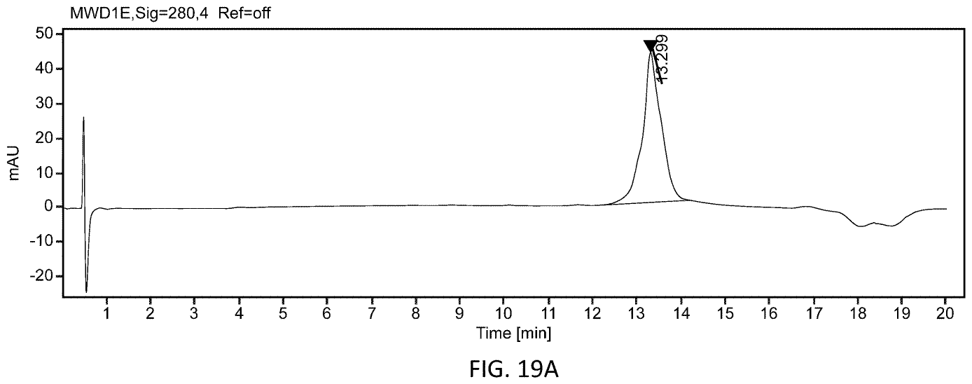

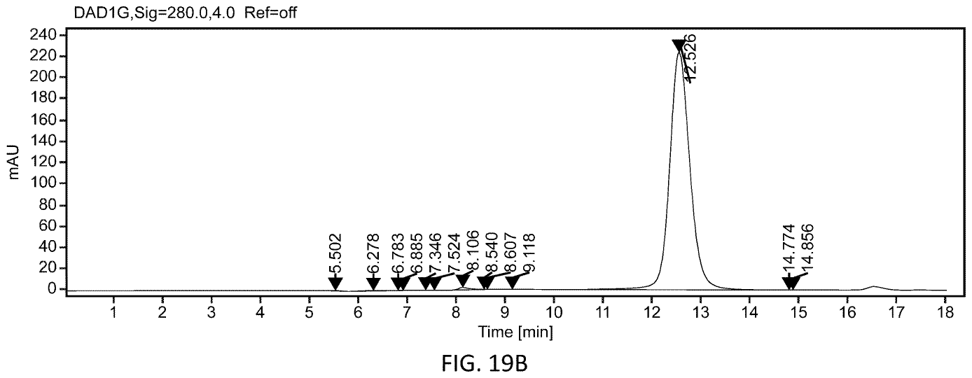

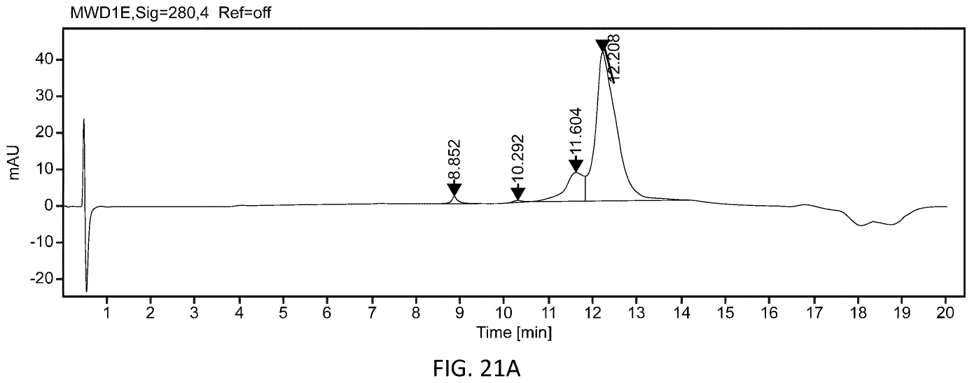

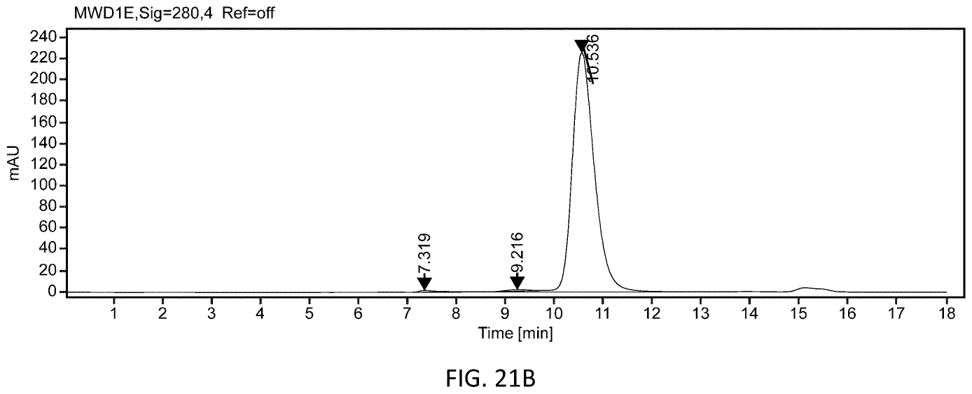

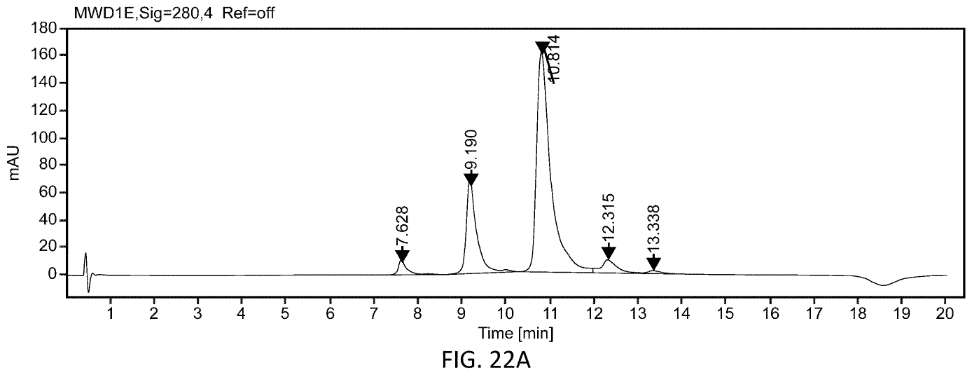

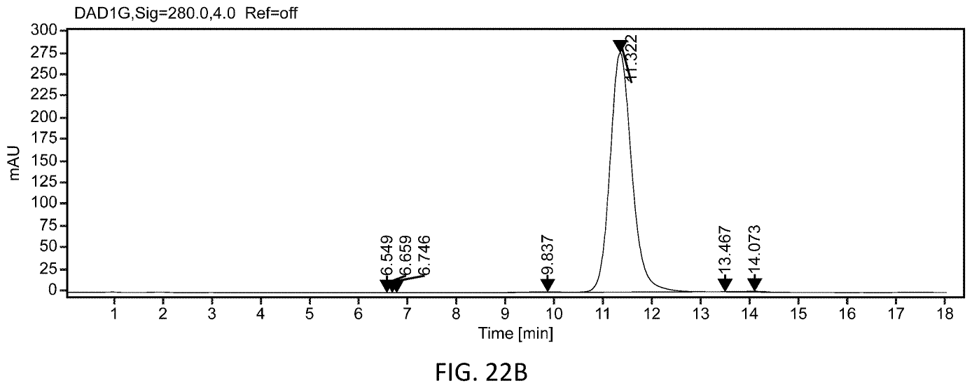

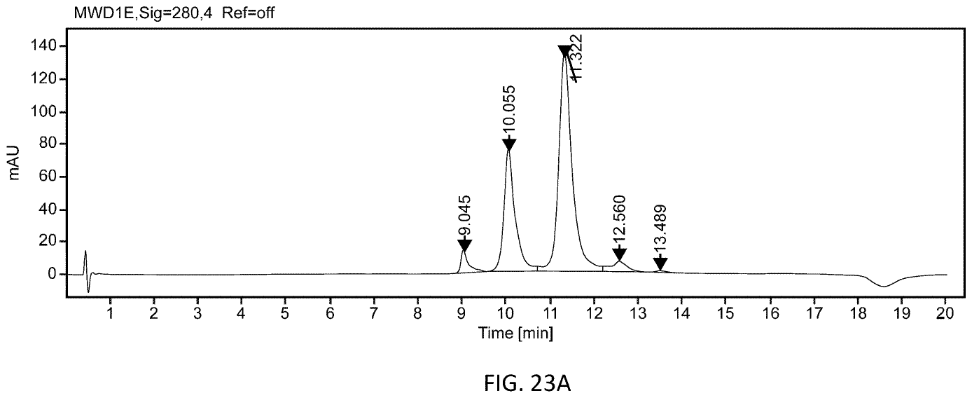

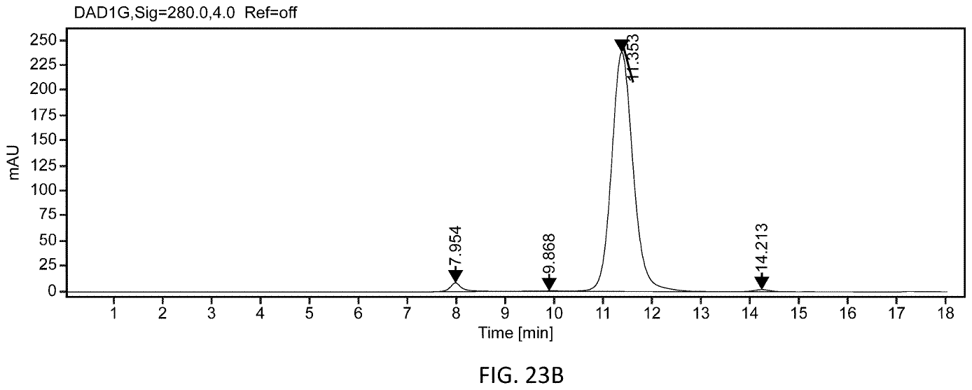

Final vc-MMAE conjugated ADCs of 11D5_Fc, 1H11-hv8_Fc, 1H11-hv8_Fc_11D5, Herceptin and Immu-132, were named as 11D5_MMAE (full name: 11D5_Fc_MMAE), 1H11_MMAE (Full name: 1H11-hv8_Fc_MMAE), BsAb-MMAE (full name: 1H11-hv8_Fc_11D5-2C_MMAE), Herceptin_MMAE and Immu-132_MMAE, respectively. The DARs of these ADCs were determined by HIC-HPLC and shown in FIGS. 19A, 20A, 21A, 22A, & 23A and the table below. The purity and stability of these ADCs were determined by SEC-HPLC and the results are shown in FIGS. 19B, 20B, 21B, 22B, & 23B and the table below. In the MMAE conjugated VHH_Fc antibodies, including the BsAb_MMAE, the majority DAR species is D4 (4 drugs conjugated) (80-100%). D3 (3 drugs conjugated) constitutes 16-18%. There was very little D2 (2 drugs conjugated) generated. There was also no D6 or D8 as only 4 Cysteines exist in the hinge regions (2 Cysteines in each hinge region) of Fc to form two disulfide-bonds, the critical structure for conjugation (each Cysteine of a disulfide-bond can conjugate one vc-MMAE). Therefore the maximum amount of drugs that can be conjugated for VHH_Fc is 4. The high percentage of D4 is ideal to maintain the strong potency of ADCs. On the other hand, the low percentage of D2 can avoid low potency, and the lack of D6/D8 can avoid potential toxicity. In contrast, in vc-MMAE conjugated traditional antibodies (Herceptin_MMAE and Immu-132_MMAE), there were high percentages of D2 and some D6 and D8 as shown in FIGS. 22A & 23A and the table below, despite performing the conjugation with special DAR4 technology to minimize the generation of D6 and D8.

TABLE 17
Analysis of vc-MMAE conjugated VHH_Fc and reference antibodies

| vc-MMAE | HIC-HPLC Drug Antibody Ratio (DAR) | | | | | | | SEC-HPLC |
|---|---|---|---|---|---|---|---|---|
| conjugated drugs | D0 | D2 | D3 | D4 | D6 | D8 | DAR | Monomer |
| 11D5_MMAE | 0.8% | 0.1% | 0.0% | 99.1% | 0 | 0 | 4.0 | 99.0% |
| 1H11_MMAE | 1.1% | 0.2% | 18.6% | 80.1% | 0 | 0 | 3.8 | 97.0% |
| BsAb_MMAE | 2.7% | 0.5% | 16.5% | 80.3% | 0 | 0 | 3.7 | 98.7% |
| Herceptin_MMAE | 3.0% | 18.5% | 0.4% | 72.9% | 3.7% | 1.3% | 3.6 | 99.3% |
| Immu-132_MMAE | 3.0% | 21.4% | 0.5% | 70.8% | 2.8% | 1.0% | 3.5 | 97.2% |

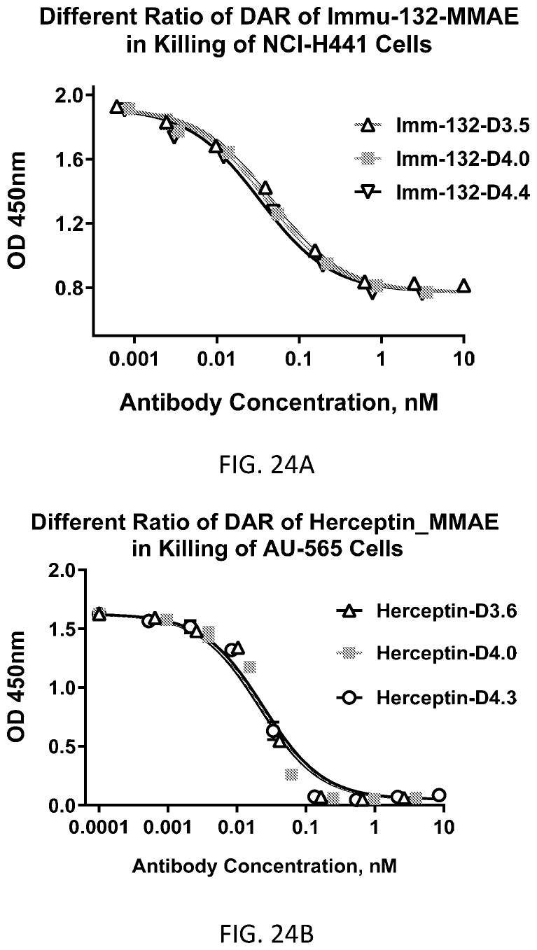

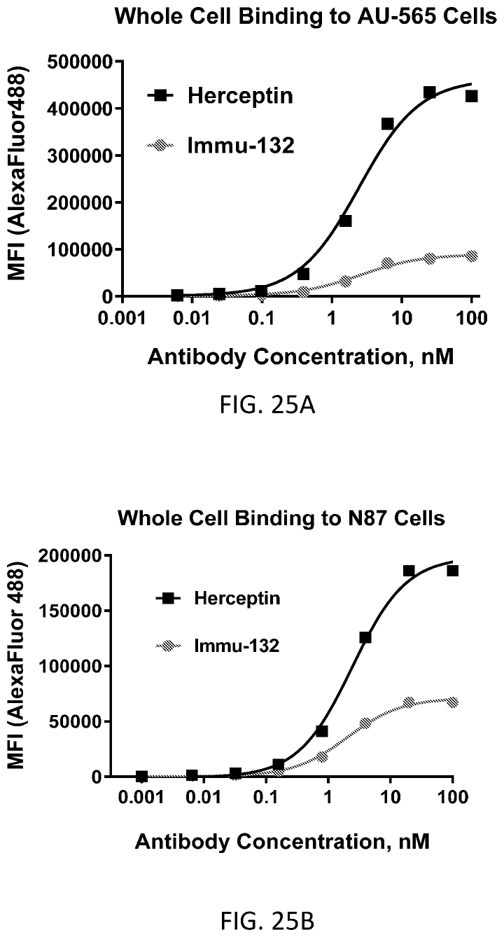

To understand the impact of ADCs with different DARs in in vitro potency, we first tested the different DARs of MMAE conjugated reference antibodies obtained during optimization of MMAE conjugation in in vitro killing assays. NCI-H441 cells expressing TROP2 at a very high level (TROP2+++, FIG. 17A) used to test killing effects of Immu-132-MMAE with DAR 3.5, 4.0, and 4.4, respectively. As shown in FIG. 24A, increasing DAR from 3.5 to 4.4 only marginally increased Immu-132_MMAE killing potency, from an IC50 of 0.047 nM to an IC50 of 0.033 nM, indicating minimum impact in cell killing caused by a higher DAR when the difference of DAR is less than 1 (Table 18). Similarly, in killing AU-565 cells expressing HER2 at a very high level (HER2+++, FIG. 25A), the potencies of Herceptin-MMAE with DAR 3.6, 4.0, and 4.3 were determined. As shown in FIG. 24B, there is no difference in potency in killing AU-565 cells by Herceptin-MMAE at different DAR levels, indicating no impact of slightly different DAR levels in in vitro potency if the difference of DAR is less than 1 (Table 18).

TABLE 18
Effects of different DAR levels of reference
ADCs in killing cancer cells

| | IC50 (nM) in ADC mediated killing | |
|---|---|---|
| | NCI-H441 cells | AU-565 cells |
| | (HER2⁺ & | (HER2⁺⁺⁺⁺ & |
| Drugs | TROP2⁺⁺⁺⁺ cells) | TROP2⁺⁺⁺ cells) |
| Herceptin_MMAE_DAR3.6 | ND | 0.024 |
| Herceptin_MMAE_DAR4.0 | ND | 0.023 |
| Herceptin_MMAE_DAR4.3 | ND | 0.021 |
| Immu-132_MMAE_DAR3.5 | 0.047 | ND |
| Immu-132_MMAE_DAR4.0 | 0.040 | ND |
| Immu-132_MMAE_DAR4.4 | 0.033 | ND |

ND: not determined.
HER2⁺: MFI 10,000; HER2⁺⁺⁺⁺: MFI 400,000, determined by binding of Herceptin at 100 nM in the presence of AlexaFluor488 conjugated anti-human IgG Fc secondary antibody in flow cytometry assays.
TROP2⁺⁺⁺ MFI 90,000; TROP2⁺⁺⁺⁺: MFI 200,000, determined by binding of Immu-132 at 100 nM in the presence of AlexaFluor488 conjugated anti-human IgG Fc secondary antibody in flow cytometry assays.

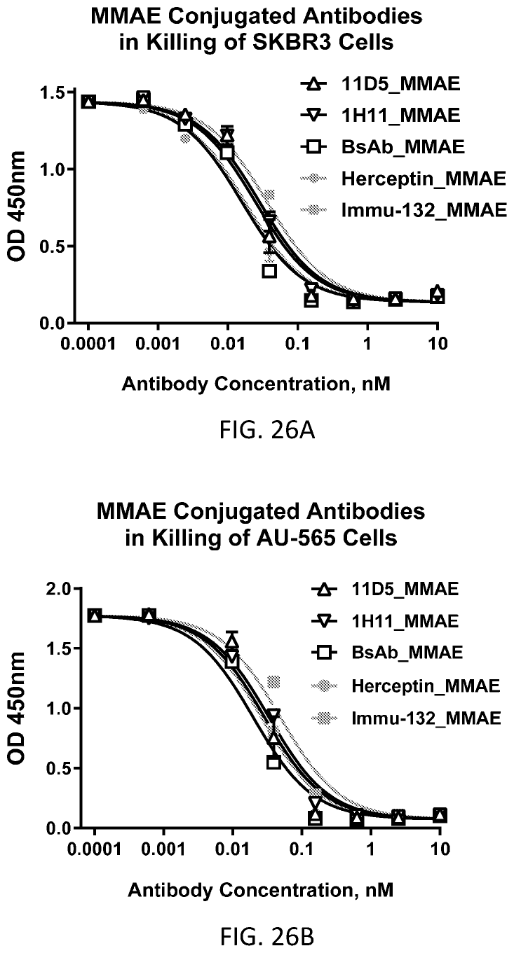

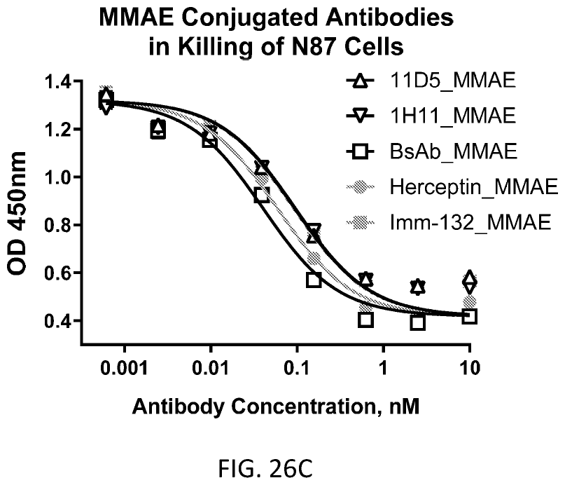

Based on the above results, if the difference of DAR level is less than 1, the comparison of potency in in vitro killing is reasonable. Therefore, comparison studies in in vitro killing of cancer cells were performed for 11D5_MMAE, 1H11_MMAE, BsAb_MMAE, Herceptin_MMAE & Immu-132_MMAE, with DAR4.0, 3.8, 3.7, 3.6 & 3.5, respectively (Table 17). As shown in FIGS. 16A, 25A & 25B, SKBR3, AU-565 and N87 cells all express HER2 at extremely high levels (HER2++++) and TROP2 at high levels (TROP2+++). The killing effects of anti-HER2-ADCs (Herceptin_MMAE and 11D5_MMAE) to these cells were very potent, with IC50 values around 0.017 nM-0.094 nM. The killing effects of anti-TROP2-ADCs (Immu-132_MMAE & 1H11_MMAE) to these cells were similar to that of anti-HER2-ADCs, with IC50 values around 0.028 nM-0.095 nM. The BsAb_MMAE is the most potent ADC among them in killing these cancer cells, with IC50 values around 0.0157 nM-0.040 nM (FIGS. 26A, 26B, 26C and Table 19).

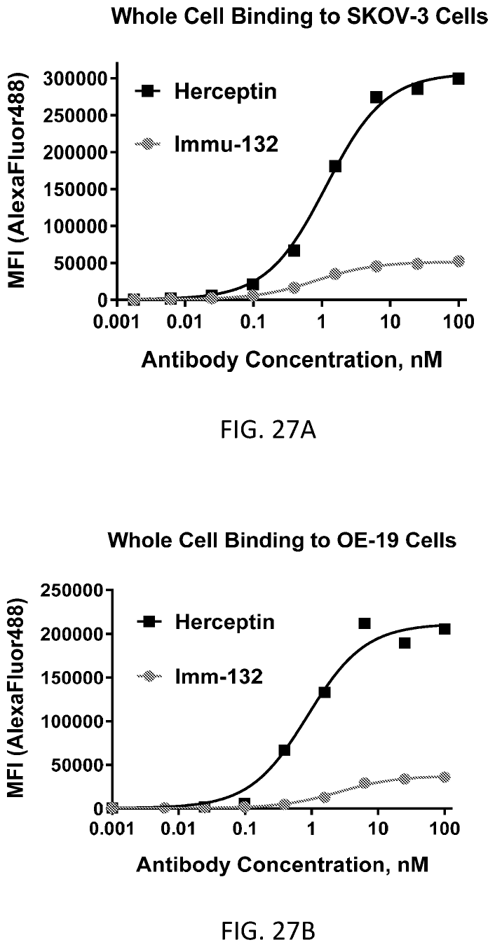

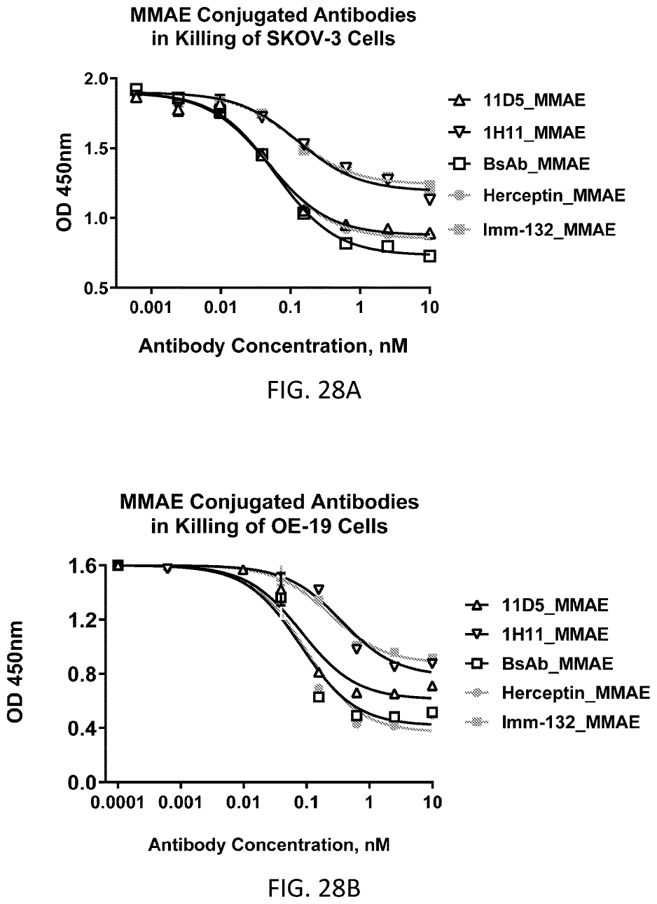

The killing effects of these ADCs to SKOV3, a human ovarian cancer cell line, and OE-19, a human esophageal adenocarcinoma cancer cell line, were also determined. Both SKOV3 and OE-19 cells express HER2 at very high or high levels with a MFI of around 300,000 or 200,000 (HER2+*** or HER2+++) as determined by the binding of Herceptin, but express TROP2 at medium levels with a MFI around 50,000 or 30,000 (TROP2++) as determined by the binding of Immu-132 (FIGS. 27A & 27B). The anti-HER2-ADC Herceptin_MMAE and 11D5_MMAE mediated potent killing of both cancer cells with IC50 values around 0.072 nM-0.111 nM. Anti-TROP2-ADC Immu-132_MMAE and 1H11_MMAE mediated weaker killing of both cancer cells compared to that of anti-HER2-ADCs, with IC50 values around 0.889 nM-1.441 nM. Again, the BsAb_MMAE is the most potent ADC among them in killing both cancer cells, with IC50 values of 0.062 nM & 0.069 nM, respectively (FIGS. 28A & 28B, and Table 19).

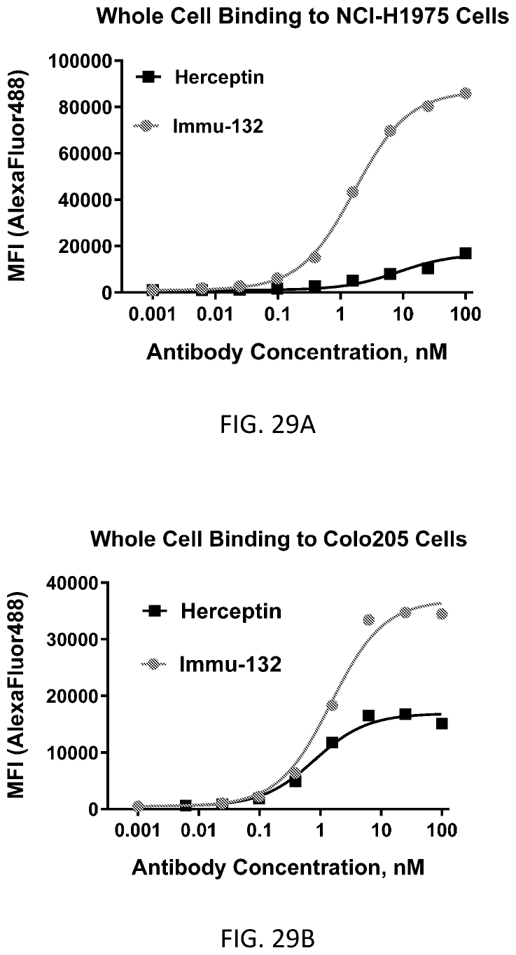

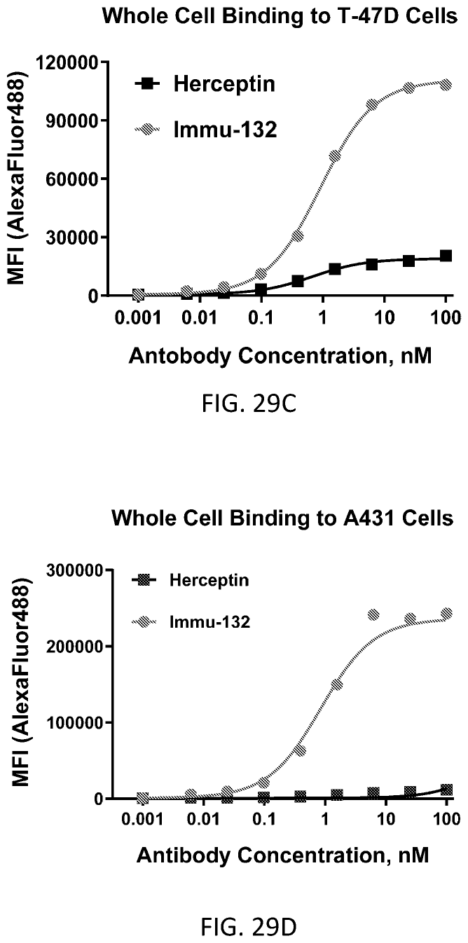

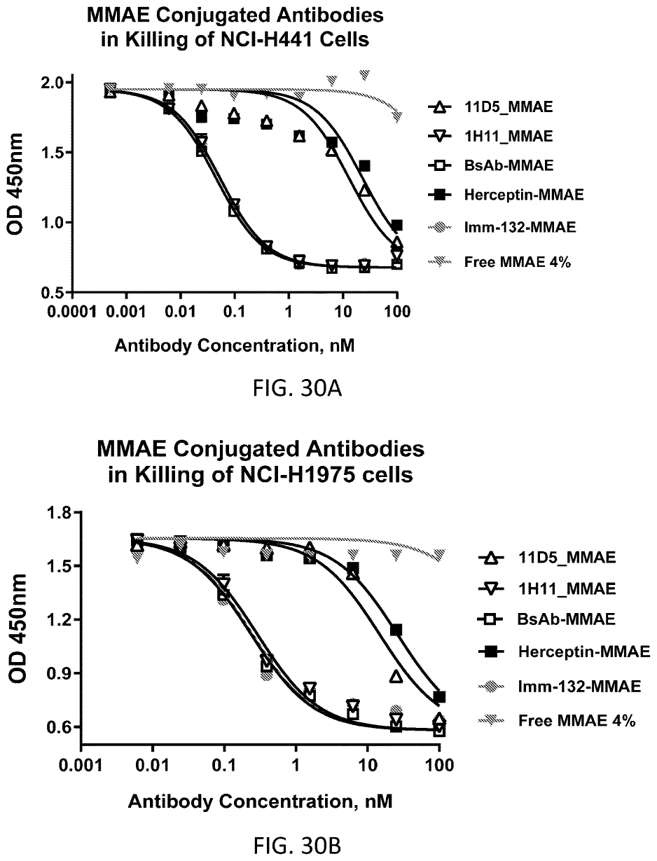

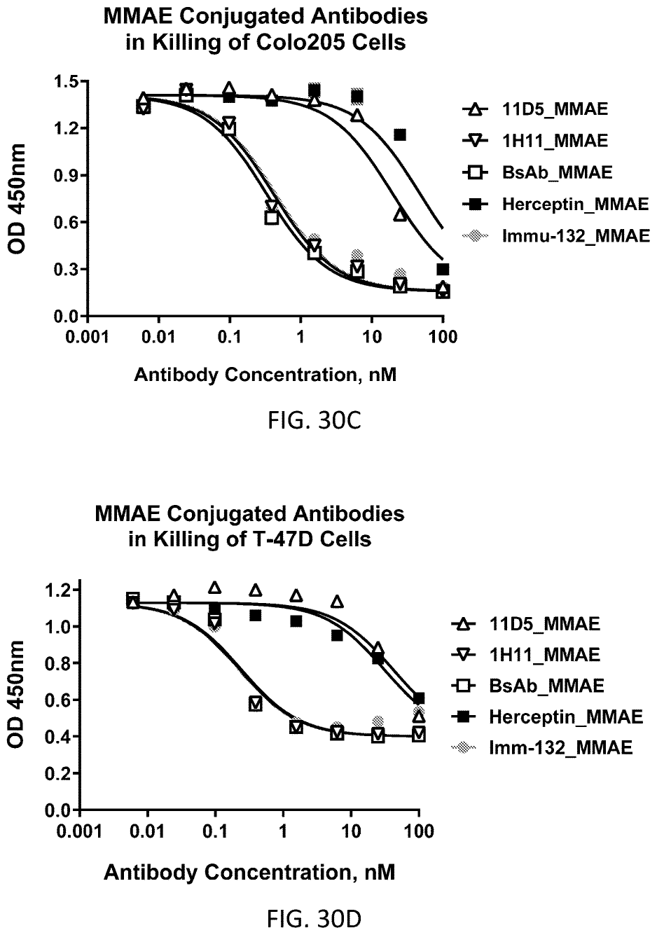

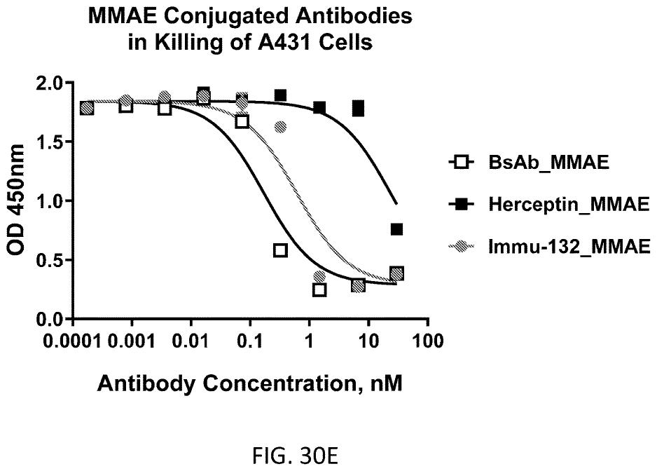

The killing effects of these ADCs to cells expressing medium high/very high TROP2 (TROP2++i+++MFI around 30,000-200,000 determined by binding of 100 nM Immu-132) but low HER2 (HER2+, MFI around 10,000-20,000 determined by binding of 100 nM Herceptin), such as NCI-H441 cells (FIG. 16A), NCI-H1975 cells (Non-small cell lung cancer cells, FIG. 29A), Colo205 cells (Colorectal cancer cells, FIG. 29B), T-47D cells (Breast cancer cells, FIG. 29C) and A431 cells (epidermoid carcinoma cancer cells, FIG. 29D) were also determined. The results are shown in FIGS. 30A, 30B, 30C, 30D, 30E) and Table 19. Anti-TROP2-ADC Immu-132_MMAE & 1H11_MMAE, but not anti-HER2_ADC Herceptin_MMAE & 11D5_MMAE, mediated potent killing effect. Again, the BsAb_MMAE is equal or more potent than the reference Immu-132_MMAE in killing these cancer cells (Table 19).

Thus, the BsAb_MMAE was able to kill cancer cells expressing HER2 and/or TROP2 at different expression levels, and is more potent and efficacious than monospecific ADCs such as Herceptin-MMAE and Immu-132_MMAE. Potential indications for the BsAb_MMAE (1H11-hv8_Fc_11D5_MMAE) include, but not limited in, many solid tumors such as Breast Cancer, Stomach Cancer, Colorectal Cancer, Lung Cancer (NSCLC & SCLC), Bladder Cancer, Cervical Cancer, Endometrial Cancer, Head & Neck Cancer, Esophageal Cancer, Pancreatic Cancer, Liver Cancer, Cholangio Cancer, Kidney Cancer, Thyroid Cancer, Skin Cancer (including Melanoma), etc., in different stages, including later stage metastatic patients.

TABLE 19
Killing effects of MMAE conjugated drugs in cancer cells expressing different levels of HER2 and TROP2

| | IC50, nM, in killing assay | | | | | | | | | |
|---|---|---|---|---|---|---|---|---|---|---|
| | | | | | | HER2⁺ & | | | | |
| | | | | | | TROP2^{++/++++} cells | | | | |
| | HER2⁺⁺⁺⁺ & TROP2⁺⁺⁺ cells | | | HER2⁺⁺⁺⁺ & TROP2⁺⁺ cells | | NCI- | NCI- | | | |
| Drugs | SKBR3 | AU-565 | N87 | SK-OV3 | OE-19 | H441 | H1975 | Colo205 | T-47D | A431 |
| 11D5_MMAE | 0.02468 | 0.02892 | 0.094 | 0.072 | 0.111 | 13.01 | 14.7 | 19.62 | 38.87 | ND |
| 1H11_MMAE | 0.02821 | 0.03359 | 0.095 | 1.211 | 0.889 | 0.054 | 0.29 | 0.42 | 0.21 | ND |
| BsAb_MMAE | 0.01574 | 0.01978 | 0.04 | 0.062 | 0.069 | 0.046 | 0.24 | 0.33 | 0.22 | 0.17 |
| Herceptin_MMAE | 0.01746 | 0.02615 | 0.059 | 0.072 | 0.074 | 23.23 | 26.1 | 46.56 | 28.58 | 23.49 |
| Immu-132_MMAE | 0.03621 | 0.04894 | 0.092 | 1.441 | 1.096 | 0.054 | 0.25 | 0.45 | 0.21 | 0.611 |

HER2⁺: MFI, 10,000-20,000; HER2⁺⁺⁺⁺: MFI, 200,000-400,000, determined by binding of Herceptin at 100 nM in the presence of AlexaFluor488 conjugated anti-human IgG Fc secondary antibody in flow cytometry assays.
TROP2⁺⁺: MFI, 30,000-50,000; TROP2⁺⁺⁺-TROP2⁺⁺⁺⁺ MFI, 65,000-250,000, determined by binding of Immu-132 at 100 nM in the presence of AlexaFluor488 conjugated anti-human IgG Fc secondary antibody in flow cytometry assays. ND: not determined.

### Example 9. In Vivo Efficacy of MMAE Conjugated Lead BsAb in a TROP2 Tumor Model

To test the efficacy of MMAE conjugated lead BsAb in a TROP2 tumor model, we selected A431, a human epidermoid carcinoma cell line, for the in vivo study, because (1) A431 cancer cells express TROP2 at a very high level, but HER2 at a very low level, as shown in FIG. 29D. (2) in vitro killing assays showed that both anti-TROP2 reference ADC (Immu-132_MMAE) and the lead BsAb-ADC (BsAb_MMAE) had potent killing effect, but not the anti-HER2 reference ADC (Herceptin_MMAE) (FIG. 30E). Therefore A431 is an ideal cancer model to determine the efficacy of anti-TROP2-ADCs.

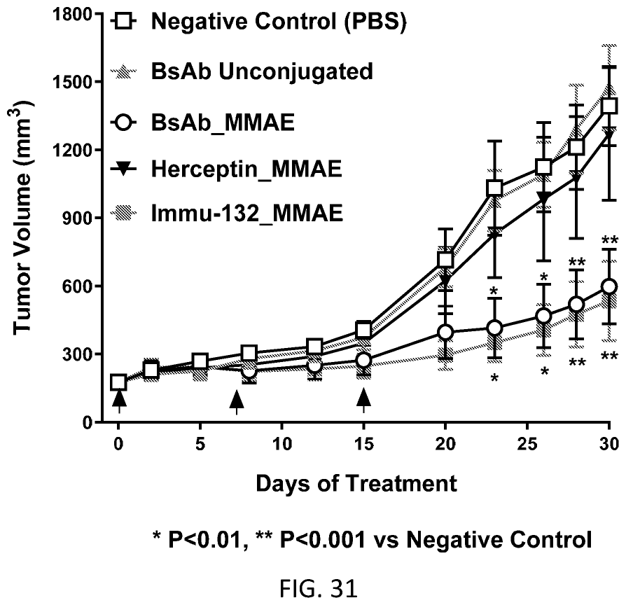

In vivo efficacy study was performed in Nu/Nu nude mice bearing A431 Epidermoid Carcinoma. A431 cancer cells (0.5×10⁶) were subcutaneously implanted 22 days before treatment. Treatment started when tumor volumes reached about 175 mm³ on average. Drugs were dosed intravenously one time per week for 3 times total at 3 mg/kg for the lead BsAb (1H11-hv8_Fc_11D5) either MMAE conjugated or unconjugated, and 4 mg/kg for Herceptin_MMAE or Immu-132_MMAE, with equal molar ratio to the BsAb_MMAE. Statistical analysis of tumor volumes (by 2-way ANOVA with Tukey's multiple comparisons test) showed that treatment with the BsAb_MMAE and Immu-132_MMAE significantly inhibited tumor growth compared to the Negative Control (the PBS treatment group) on day 23 post-treatment and thereafter (FIG. 31). In contrast, treatment with Herceptin_MMAE and unconjugated BsAb did not have significant impact on tumor growth compared to the Negative Control. Both BsAb_MMAE and Immun-132_MMAE significantly reduced tumor growth compared to the Negative Control with tumor growth inhibition (TGI) of 60.8% and 66.6%, respectively on day 30 post-treatment.

No significant change of body weight was observed among mice from different treatment groups, indicating drugs were well-tolerated.

### Example 10. In Vivo Efficacy of MMAE Conjugated Lead BsAb in a HER2 Tumor Model

To test the efficacy of MMAE conjugated lead BsAb in a HER2 tumor model, we selected OE-19, a human esophageal adenocarcinoma cell line, for the in vivo study. As shown in FIG. 27B, OE-19 cancer cells express HER2 at a very high level, but TROP2 at a medium-low level. In vitro killing assays also showed that both anti-HER2 reference ADC (Herceptin_MMAE) and the lead BsAb-ADC had potent killing effects to OE-19 cells, but the anti-TROP2 reference ADC (Immu-132_MMAE) had weaker killing effects (FIG. 28B, & Table 19). These results suggest that the OE-19 can be used as a cancer model to determine the efficacy of the anti-HER2 arm of ADCs, with limited impact from the anti-TROP2 arm.

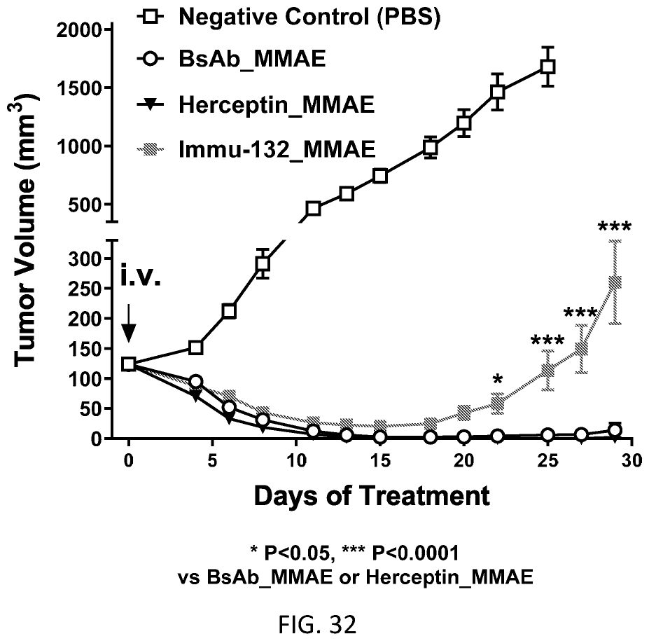

In vivo efficacy study was performed in the immunodeficient B-NDG mice (generated in Biocytogen by deleting the IL2rg gene from NOD-scid mice with severe immunodeficiency phenotype) bearing the OE-19 tumor. OE-19 cancer cells (0.5×10⁶ cells in 0.1 ml) were subcutaneously implanted. Treatment started 10 days later when tumor volumes reached about 124 mm³ on average. Drugs were dosed intravenously one time at 5 mg/kg for ADCs, with PBS as a Negative Control. All treatment groups significantly reduced tumor growth compared to the Negative Control as shown in FIG. 32. However, tumors gradually grow up observed in Immu-132_MMAE treatment group but not in BsAb_MMAE or Herceptin_MMAE treatment groups. The difference of tumor volumes between treatment with Immu-132_MMAE and BsAb_MMAE or Herceptin_MMAE reached significant on 22 days of treatment (P<0.05) and was more pronounced thereafter (P<0.0001, by two way ANOVA with Tukey's multiple comparisons test).

No significant change of body weight was observed among mice from different treatment groups, indicating drugs were well-tolerated.

### Example 11. Conjugation of Different Types of Payloads to Anti-HER2/TROP2 Bispecific Antibody with Different DAR Ratios

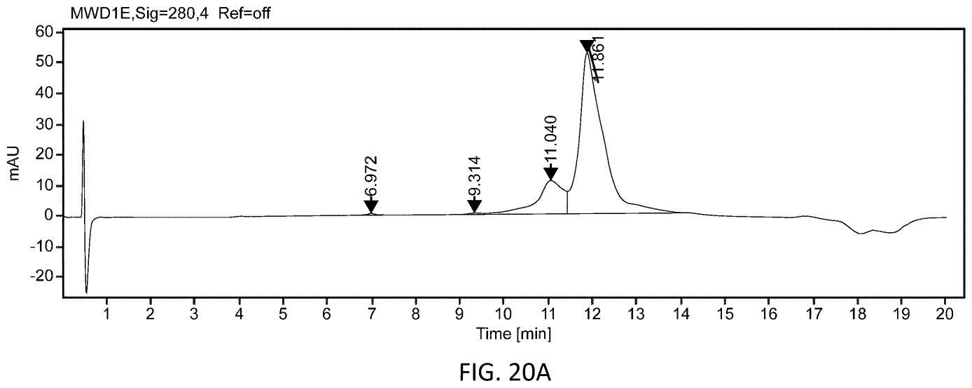

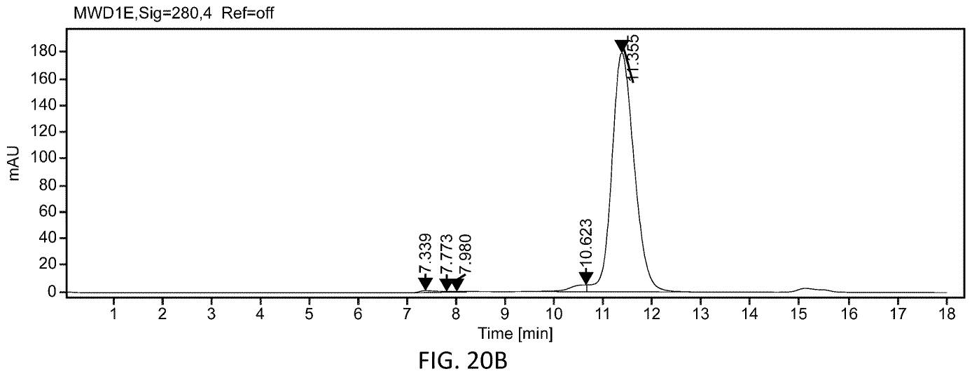

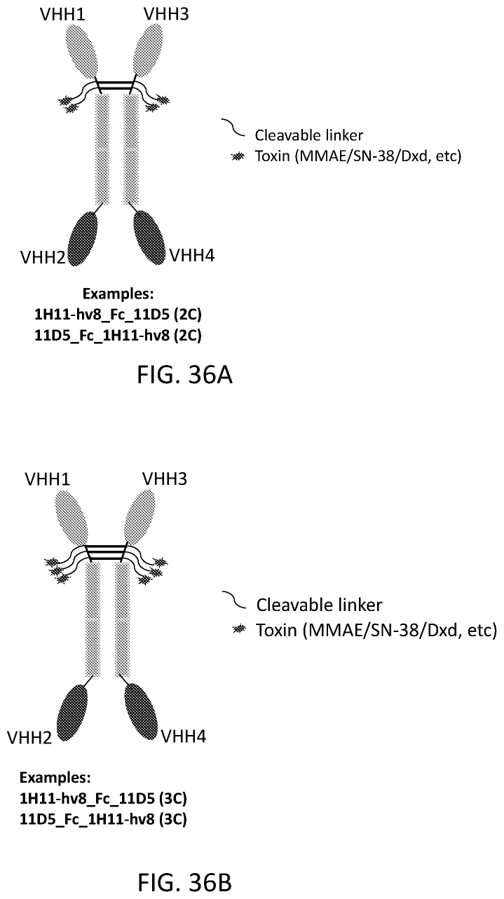

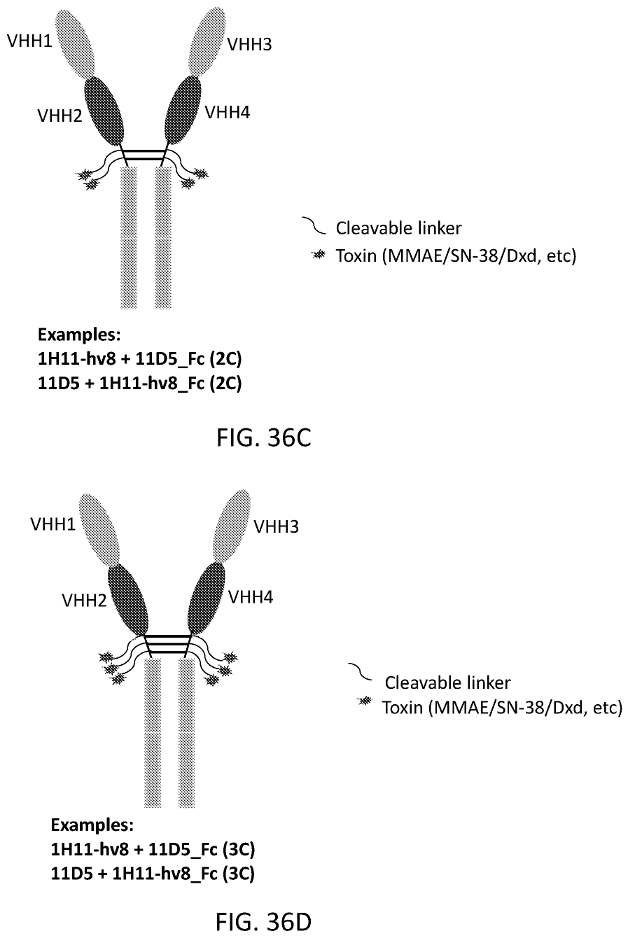

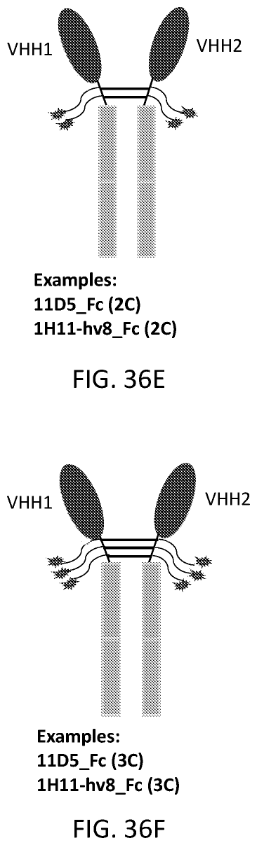

In the above experiments, the payload selected for ADC conjugation is MMAE, which is a potent mitotic inhibitor by inhibiting micro-tubulin. However, several different types of toxins/payloads with different potencies and mode of actions are also available for antibody-drug-conjugates, such as SN-38 and Dxd. Both SN-38 and Dxd are Topoisomerase I inhibitors, with potencies in inhibiting cancer cells 100-1000 times weaker than MMAE in vitro. The potential advantage of SN-38 and/or Dxd conjugated antibodies is that there may be less toxicity or unwanted side effects when compared to that of MMAE conjugated antibodies. However, because the potencies of SN-38 and Dxd are much weaker than that of MMAE, a higher amount of payload/drug is needed. Therefore, a construct of anti-HER2/TROP2 BsAb with 3 Cysteines in each hinge region to form 3 disulfide-bonds was constructed, by removing the C220S mutation. The construct was named 1H11-hv8_Fc-11D5-3C. The sequence is shown in the table below. In the construct with 2 Cysteines (with C220S) in each hinge region which only forms 2 disulfide-bonds, 4 payloads (drugs) can be conjugated (FIG. 36A), leading to a maximum DAR of 4.0. Examples of the vc-MMAE conjugated VHH-Fc antibodies with 2 Cysteines are shown in FIGS. 19A, 20A & 21A, and Table 17, the DAR ranges of these antibodies are 3.87-4.0. This format can be used for BsAb-ADC targeting any Tumor Associated Antigens. In the construct with 3 Cysteines in each hinge region, 6 payloads can be conjugated (FIG. 36B), leading to a maximum DAR of 6.0. Similar strategies can be used for BsAb-ADCs with tandem formats, as shown in FIG. 36C & 36D, or monospecific VHH_Fc-ADCs, as shown in FIG. 36E & 36F. A cleavable linker, such as mv-vc-PABC or CL2-PABC, or a non-cleavable linker, can be used. Also, different types of payloads such as MMAE, SN-38 and Dxd can be used.

TABLE 20
Sequence of the BsAb with 3 Cysteines in each hinge region

| Clone Name of Bispecific Antibody | | | | Full |
|---|---|---|---|---|
| 1H11-hv8_Fc_11D5-3C | | | | Length |
| 1H11-hv8 | Hinge (3C) + CH2 + CH3 (IgG1) | Linker2 | 11D5 | SEQ ID |
| EVQLVESGGGLVQPG | EPKSCDKTHTCPPCPAPELLGGPSVFLFP | GSGGSG | QVQLVESGGGVV | 22 |
| GSLRLSCAASGGTFS | PKPKDTLMISRTPEVTCVVVDVSHEDPEV | GSGGSG | QPGRSLRLSCAA | |
| RYIMGWFRQAPGKER | KFNWYVDGVEVHNAKTKPREEQYNSTYRV | GSG | SGSTFSSLAMHW | |
| EFVSTISGSGGSTYY | VSVLTVLHQDWLNGKEYKCKVSNKALPAP | (SEQ ID | VRQAPGKERELV | |
| ADSVKGRFTISRDNS | IEKTISKAKGQPREPQVYTLPPSREEMTK | NO: 35) | AAISYSGGYTYY | |
| KNTLYLQMNSLRAED | NQVSLTCLVKGFYPSDIAVEWESNGQPEN | | ADSVKGRFTISR | |
| TAVYYCASTFYSTTY | NYKTTPPVLDSDGSFFLYSKLTVDKSRWQ | | DNSKNTLYLQMN | |
| VAQRDARDYAYWGQG | QGNVFSCSVMHEALHNHYTQKSLSLSPGK | | SLRAEDTAVYYC | |
| TQVTVSS | (SEQ ID NO: 37) | | AASSIFIGAYDY | |
| (SEQ ID NO: 10) | | | WGQGTLVTVSS | |
| | | | (SEQ ID | |
| | | | NO: 1) | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody or antigen-binding fragment thereof that specifically binds to HER2 (human epidermal growth factor receptor 2), comprising:
a heavy-chain antibody variable domain (VHH) comprising complementarity determining regions (CDRs) 1, 2, and 3,
wherein the CDRs 1, 2, 3 amino acid sequences are set forth in SEQ ID NOs: 22, 23, and 24, respectively.

2. (canceled)

3. The antibody or antigen-binding fragment thereof of claim 1, wherein the VHH comprises an amino acid sequence that is at least 80% identical to a selected VHH sequence, wherein the selected VHH sequence is set forth in SEQ ID NO: 1.

4.-9. (canceled)

10. An antibody or antigen-binding fragment thereof that specifically binds to TROP2 (Tumor-associated calcium signal transducer 2), comprising:
a heavy-chain antibody variable domain (VHH) comprising complementarity determining regions (CDRs) 1, 2, and 3,
wherein the CDRs 1, 2, and 3 amino acid sequences are one of the following:
(1) the CDRs 1, 2, 3 amino acid sequences are set forth in SEQ ID NOs: 25, 26, and 27, respectively;
(2) the CDRs 1, 2, 3 amino acid sequences are set forth in SEQ ID NOs: 28, 29, and 30, respectively; and
(3) the CDRs 1, 2, 3 amino acid sequences are set forth in SEQ ID NOs: 31, 32, and 33, respectively.

11. The antibody or antigen-binding fragment thereof of claim 10, wherein the VHH comprises CDRs 1, 2, 3 with the amino acid sequences set forth in SEQ ID NOs: 25, 26, and 27, respectively.

12. The antibody or antigen-binding fragment thereof of claim 10, wherein the VHH comprises CDRs 1, 2, 3 with the amino acid sequences set forth in SEQ ID NOs: 28, 29, and 30, respectively.

13. The antibody or antigen-binding fragment thereof of claim 10, wherein the VHH comprises CDRs 1, 2, 3 with the amino acid sequences set forth in SEQ ID NOs: 31, 32, and 33, respectively.

14. The antibody or antigen-binding fragment thereof of claim 10, wherein the VHH comprises an amino acid sequence that is at least 80% identical to a selected VHH sequence, wherein the selected VHH sequence is selected from the group consisting of SEQ ID NOs: 2-12.

15.-17. (canceled)

18. The antibody or antigen-binding fragment thereof of claim 14, wherein the VHH comprises the sequence of SEQ ID NO: 10.

19.-49. (canceled)

50. A polypeptide complex, comprising
(a) a first polypeptide comprising from N-terminus to C-terminus: a VHH2, a first hinge region, a first CH2, a first CH3, and VHH1; and
(b) a second polypeptide comprising from N-terminus to C-terminus: a VHH4, a second hinge region, a second CH2, a second CH3, and a VHH3,
wherein the VHH1 and the VHH3 specifically bind to HER2, and the VHH2 and the VHH4 specifically bind to TROP2.

51. The polypeptide complex of claim 50, wherein the first polypeptide comprises a sequence that is at least 80% identical to SEQ ID NO: 16 or 20; and/or wherein the second polypeptide comprises a sequence that is at least 80% identical to SEQ ID NO: 16 or 20.

52.-54. (canceled)

55. The polypeptide complex of claim 50, wherein the VHH1 and/or the VHH3 comprise complementarity determining regions (CDRs) 1, 2, and 3, wherein the CDR1 region comprises an amino acid sequence that is identical to a selected CDR1 amino acid sequence, the CDR2 region comprises an amino acid sequence that is identical to a selected CDR2 amino acid sequence, and the CDR3 region comprises an amino acid sequence that is identical to a selected CDR3 amino acid sequence; wherein the selected CDRs 1, 2, and 3 amino acid sequences are are set forth in SEQ ID NOs: 22, 23, and 24, respectively.

56. (canceled)

57. The polypeptide complex of claim 50, wherein the VHH2 and/or the VHH4 comprise complementarity determining regions (CDRs) 1, 2, and 3, wherein the CDR1 region comprises an amino acid sequence that is identical to a selected CDR1 amino acid sequence, the CDR2 region comprises an amino acid sequence that is identical to a selected CDR2 amino acid sequence, and the CDR3 region comprises an amino acid sequence that is identical to a selected CDR3 amino acid sequence; wherein the selected CDRs 1, 2, and 3 amino acid sequences are one of the following:
(1) the selected CDRs 1, 2, 3 amino acid sequences are set forth in SEQ ID NOs: 25, 26, and 27, respectively:
(2) the selected CDRs 1, 2, 3 amino acid sequences are set forth in SEQ ID NOs: 28, 29, and 30, respectively; and
(3) the selected CDRs 1, 2, 3 amino acid sequences are set forth in SEQ ID NOs: 31, 32, and 33, respectively.

58. (canceled)

59. A nucleic acid comprising a polynucleotide encoding the antibody or antigen-binding fragment thereof of claim 10.

60. (canceled)

61. A cell comprising one or more of the nucleic acids of claim 59.

62. A method of producing an antibody or an antigen-binding fragment thereof, the method comprising
(a) culturing the cell of claim 61 under conditions sufficient for the cell to produce the antibody or the antigen-binding fragment; and
(b) collecting the antibody or the antigen-binding fragment produced by the cell.

63.-65. (canceled)

66. An antibody-drug conjugate comprising the polypeptide complex of claim 50, covalently bound to a therapeutic agent.

67. (canceled)

68. A method of treating a subject having cancer, the method comprising administering a therapeutically effective amount of a composition comprising the antibody or antigen-binding fragment thereof of claim 10, to the subject.

69.-91. (canceled)

92. The ADC of claim 66, wherein each of the first hinge region and the second hinge region consists of 2 cysteines, wherein the drug-to-antibody ratio (DAR) is 1.0-4.0.

93. The ADC of claim 66, wherein each of the first hinge region and the second hinge region consists of 3 cysteines, wherein the drug-to-antibody ratio (DAR) is 4.0-6.0.

94. A method of diagnosing a disease or condition, wherein the method comprises incubating a sample with a composition comprising the antibody or antigen-binding fragment thereof of claim 10.


SEQUENCE LISTING (39 sequences)

SEQ ID NO: 1 - protein, 119 aa, synthetic construct
QVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSS

SEQ ID NO: 2 - protein, 129 aa, synthetic construct
LVQLVESGGGLVQAGGSQRLSCAVSGRAFAYYAMGWFRQAPGKEREFVAAIAGSGDSTYYADSVKGRFTISRDYAKNTNYLQMNSLKPEDTAVYYCAAAGGTFYSGTAVATRSSQYDYWGQGTQVTVSS

SEQ ID NO: 3 - protein, 129 aa, synthetic construct
QVQVVESGGGLVQAGGSQRLSCAVSGHPFAMYAMGWFRQAPGKEREFVAAIAGNGGSTSYADSVKGRFTISRDYAKNTNYLQMNSLEPEDTAVYYCAAAGGTFYSGTTVATRSSHYDYWGQGTQVTVSS

SEQ ID NO: 4 - protein, 127 aa, synthetic construct
LVQLVESGGGLVQAGDSLRLSCTASGGTFSRYIIGWFRQAPGKEREFVTTISGSGGSTYYADSVKGRFTISRDNAKNTVFLQMNSLKPEDTAVYYCASTFYSTTYVAQRDARDYAYWGQGTQVTVSS

SEQ ID NO: 5 - protein, 129 aa, synthetic construct
EVQLVESGGGLVQPGGSLRLSCAASGHPFAMYAMGWFRQAPGKEREFVSAIAGNGGSTSYADSVKGRFTISRDNSKNTLYLQMNSLRAEDTAVYYCAAAGGTFYSGTTVATRSSHYDYWGQGTQVTVSS

SEQ ID NO: 6 - protein, 129 aa, synthetic construct
EVQLVESGGGLVQPGGSLRLSCAASGHPFAMYAMGWFRQAPGKEREFVSAIAGNGGSTYYADSVKGRFTISRDNSKNTLYLQMNSLRAEDTAVYYCAAAGGTFYSGTTVATRSSHYDYWGQGTQVTVSS

SEQ ID NO: 7 - protein, 129 aa, synthetic construct
EVQLVESGGGLVQPGGSLRLSCAASGHPFAMYAMGWFRQAPGKEREFVAAIAGNGGSTSYADSVKGRFTISRDNSKNTLYLQMNSLRAEDTAVYYCAAAGGTFYSGTTVATRSSHYDYWGQGTQVTVSS

SEQ ID NO: 8 - protein, 127 aa, synthetic construct
EVQLVESGGGLVQPGGSLRLSCAASGGTFSRYIIGWFRQAPGKEREFVTTISGSGGSTYYADSVKGRFTISRDNSKNTLYLQMNSLRAEDTAVYYCASTFYSTTYVAQRDARDYAYWGQGTQVTVSS

SEQ ID NO: 9 - protein, 127 aa, synthetic construct
EVQLVESGGGLVQPGGSLRLSCAASGGTFSRYIIGWFRQAPGKEREFVSTISGSGGSTYYADSVKGRFTISRDNSKNTLYLQMNSLRAEDTAVYYCASTFYSTTYVAQRDARDYAYWGQGTQVTVSS

SEQ ID NO: 10 - protein, 127 aa, synthetic construct
EVQLVESGGGLVQPGGSLRLSCAASGGTFSRYIMGWFRQAPGKEREFVSTISGSGGSTYYADSVKGRFTISRDNSKNTLYLQMNSLRAEDTAVYYCASTFYSTTYVAQRDARDYAYWGQGTQVTVSS

SEQ ID NO: 11 - protein, 127 aa, synthetic construct
EVQLVESGGGLVQPGGSLRLSCAASGGTFSRYIMSWFRQAPGKEREFVSTISGSGGSTYYADSVKGRFTISRDNSKNTLYLQMNSLRAEDTAVYYCASTFYSTTYVAQRDARDYAYWGQGTQVTVSS

SEQ ID NO: 12 - protein, 127 aa, synthetic construct
EVQLVESGGGLVQPGGSLRLSCAASGGTFSRYIMSWFRQAPGKEREFVSAISGSGGSTYYADSVKGRFTISRDNSKNTLYLQMNSLRAEDTAVYYCASTFYSTTYVAQRDARDYAYWGQGTQVTVSS

SEQ ID NO: 13 - protein, 351 aa, synthetic construct
QVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 359 aa, synthetic construct
EVQLVESGGGLVQPGGSLRLSCAASGGTFSRYIMSWFRQAPGKEREFVSAISGSGGSTYYADSVKGRFTISRDNSKNTLYLQMNSLRAEDTAVYYCASTFYSTTYVAQRDARDYAYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 490 aa, synthetic construct
EVQLVESGGGLVQPGGSLRLSCAASGGTFSRYIMSWFRQAPGKEREFVSAISGSGGSTYYADSVKGRFTISRDNSKNTLYLQMNSLRAEDTAVYYCASTFYSTTYVAQRDARDYAYWGQGTQVTVSSGSGGSGGSGGSGQVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 493 aa, synthetic construct
EVQLVESGGGLVQPGGSLRLSCAASGGTFSRYIMSWFRQAPGKEREFVSAISGSGGSTYYADSVKGRFTISRDNSKNTLYLQMNSLRAEDTAVYYCASTFYSTTYVAQRDARDYAYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSS

SEQ ID NO: 17 - protein, 490 aa, synthetic construct
QVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSSGSGGSGGSGGSGEVQLVESGGGLVQPGGSLRLSCAASGGTFSRYIMSWFRQAPGKEREFVSAISGSGGSTYYADSVKGRFTISRDNSKNTLYLQMNSLRAEDTAVYYCASTFYSTTYVAQRDARDYAYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 493 aa, synthetic construct
QVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGEVQLVESGGGLVQPGGSLRLSCAASGGTFSRYIMSWFRQAPGKEREFVSAISGSGGSTYYADSVKGRFTISRDNSKNTLYLQMNSLRAEDTAVYYCASTFYSTTYVAQRDARDYAYWGQGTQVTVSS

SEQ ID NO: 19 - protein, 359 aa, synthetic construct
EVQLVESGGGLVQPGGSLRLSCAASGGTFSRYIMGWFRQAPGKEREFVSTISGSGGSTYYADSVKGRFTISRDNSKNTLYLQMNSLRAEDTAVYYCASTFYSTTYVAQRDARDYAYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 493 aa, synthetic construct
EVQLVESGGGLVQPGGSLRLSCAASGGTFSRYIMGWFRQAPGKEREFVSTISGSGGSTYYADSVKGRFTISRDNSKNTLYLQMNSLRAEDTAVYYCASTFYSTTYVAQRDARDYAYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSS

SEQ ID NO: 21 - protein, 490 aa, synthetic construct
EVQLVESGGGLVQPGGSLRLSCAASGGTFSRYIMGWFRQAPGKEREFVSTISGSGGSTYYADSVKGRFTISRDNSKNTLYLQMNSLRAEDTAVYYCASTFYSTTYVAQRDARDYAYWGQGTQVTVSSGSGGSGGSGGSGQVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 493 aa, synthetic construct
EVQLVESGGGLVQPGGSLRLSCAASGGTFSRYIMGWFRQAPGKEREFVSTISGSGGSTYYADSVKGRFTISRDNSKNTLYLQMNSLRAEDTAVYYCASTFYSTTYVAQRDARDYAYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGSGGSGGSGGSGQVQLVESGGGVVQPGRSLRLSCAASGSTFSSLAMHWVRQAPGKERELVAAISYSGGYTYYADSVKGRFTISRDNSKNTLYLQMNSLRAEDTAVYYCAASSIFIGAYDYWGQGTLVTVSS

SEQ ID NO: 23 - protein, 8 aa, synthetic construct
ISYSGGYT

SEQ ID NO: 24 - protein, 12 aa, synthetic construct
AASSIFIGAYDY

SEQ ID NO: 25 - protein, 8 aa, synthetic construct
GRAFAYYA

SEQ ID NO: 26 - protein, 8 aa, synthetic construct
IAGSGDST

SEQ ID NO: 27 - protein, 22 aa, synthetic construct
AAAGGTFYSGTAVATRSSQYDY

SEQ ID NO: 28 - protein, 8 aa, synthetic construct
GHPFAMYA

SEQ ID NO: 29 - protein, 8 aa, synthetic construct
IAGNGGST

SEQ ID NO: 30 - protein, 22 aa, synthetic construct
AAAGGTFYSGTTVATRSSHYDY

SEQ ID NO: 31 - protein, 8 aa, synthetic construct
GGTFSRYI

SEQ ID NO: 32 - protein, 8 aa, synthetic construct
ISGSGGST

SEQ ID NO: 33 - protein, 20 aa, synthetic construct
ASTFYSTTYVAQRDARDYAY

SEQ ID NO: 34 - protein, 12 aa, synthetic construct
GSGGSGGSGGSG

SEQ ID NO: 35 - protein, 15 aa, synthetic construct
GSGGSGGSGGSGGSG

SEQ ID NO: 36 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 5 aa, synthetic construct
  REGION >5: note = The entire sequence of amino acids 1-5 can be repeated one to eight times
GGGGS

SEQ ID NO: 39 - protein, 8 aa, synthetic construct
GSTFSSLA